(19) United States
(12) Patent Application Publication
(10) Pub. No.: US 2024/0294504 A1
(43) Pub. Date: Sep. 5, 2024

(54) COMPOUNDS, COMPOSITIONS, AND METHODS FOR INDUCING ANTIMICROBIAL INTRACELLULAR ACTIVITY AND FOR PREVENTING AND TREATING MICROBIAL INFECTIONS

(71) Applicants: The Broad Institute, Inc., Cambridge, MA (US); THE GENERAL HOSPITAL CORPORATION, Boston, MA (US)

(72) Inventors: Deborah HUNG, Cambridge, MA (US); Christoph ERNST, Boston, MA (US); Tomohiko KAWATE, Boston, MA (US)

(73) Assignees: The Broad Institute, Inc., Cambridge, MA (US); THE GENERAL HOSPITAL CORPORATION, Boston, MA (US)

(21) Appl. No.: 18/663,765

(22) Filed: May 14, 2024

Related U.S. Application Data
(63) Continuation of application No. PCT/US2022/049952, filed on Nov. 15, 2022.
(60) Provisional application No. 63/279,559, filed on Nov. 15, 2021.

(51) Int. Cl. C07D 401/14; A61K 31/085; A61K 31/135; A61K 31/40; A61K 31/415; A61K 31/454; A61K 31/4545; A61K 31/505; A61P 31/04; C07C 43/225; C07D 207/34; C07D 213/73; C07D 213/74; C07D 231/12; C07D 239/26; C07D 401/04

(52) U.S. Cl. CPC C07D 401/14; A61K 31/085; A61K 31/135; A61K 31/40; A61K 31/415; A61K 31/454; A61K 31/4545; A61K 31/505; A61P 31/04; C07C 43/225; C07D 207/34; C07D 213/73; C07D 213/74; C07D 231/12; C07D 239/26; C07D 401/04

(57) ABSTRACT

Compounds, pharmaceutical compositions, and methods of use of said compounds and pharmaceutical compositions for treating or preventing a microbial infection or for inducing antimicrobial activity against a microbial infection.

### CROSS-REFERENCE TO RELATED APPLICATIONS

This application is a continuation of International Application No. PCT/US2022/049952, filed Nov. 15, 2022, which claims priority under 35 U.S.C. § 119(e) of U.S. Provisional Application No. 63/279,559, filed Nov. 15, 2021, the contents of each of which are hereby incorporated by reference into this application in their entireties.

### TECHNICAL FIELD

The subject matter disclosed herein is generally directed to compounds, pharmaceutical formulations thereof, and methods of use thereof for inducing intracellular antimicrobial activity in cells, such as, for example, human cells, and preventing and treating microbial infections in in subjects, such as, for example, human subjects.

### BACKGROUND

Antibiotic resistance is increasingly becoming a global threat to public health due to the emergence of multidrug-resistant bacteria and the sharp decline in antibiotic discovery, which is especially concerning for the treatment of common hospital-associated Gram-negative infections. Less appreciated is the fact that antibiotic treatment can be ineffective in the absence of antibiotic resistance and result in persistent infection. Symptomatic persistent infections can have an immediate impact on morbidity and mortality, while asymptomatic persistent infections can cause recurring potentially disseminating acute infections. Multiple factors appear to contribute to persistent infection, such as an immunocompromised state, comorbidities, bacterial factors, and intrinsic properties of antibiotics which limit their efficacy in the host. For example, many antibiotics are only effective against replicating bacteria and are unable to penetrate host tissues to treat deep seated infections⁴. Indeed, many bacterial pathogens invade, grow, and persist in host cells, where they are protected from various components of the immune system, and where antibiotic efficacy can be limited due to low antibiotic penetrance and non-growing subpopulations⁴. Thus, not only is there an urgent need to develop new treatment approaches to treat antibiotic resistant infections but also to generally treat bacterial infections more effectively to prevent persistent infections.

### SUMMARY

In one aspect, the present invention provides compounds for use in pharmaceutical compounds and methods for treating or preventing a microbial infection or for inducing anti-microbial activity against a microbial infection. In an example embodiment, a compound or a salt thereof has the structural formula:

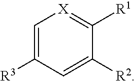

where X is C or N;
where R¹ is a hydrogen group, an amino group, an alkyl (including cycloalkyl) amino group, an alkyl azide (including a cycloalkyl azide) group, an alkylaryl (including cycloalkylaryl) amino group, an alkylaryloxy (including cycloalkylaryloxy) group, or an aryl amino group, and where R¹ is optionally independently substituted at one or more aryl ring positions with one or more halogen groups;
where R² is a hydrogen group, a hydroxyl group, an alkoxy (including cycloalkoxy) group, an alkynyl alkoxy (including alkynyl cycloalkoxy) group, or an aryl alkoxy (including aryl cycloalkoxy) group, and where, optionally, the aryl alkoxy (including aryl cycloalkoxy) group is independently substituted at one or more aryl ring positions with one or more halogen groups; where R³ is a halogen group or a ring carbon of an aromatic heterocyclic amine group, and where, optionally, the aromatic heterocyclic amine group is independently substituted at one or more heterocyclic ring positions with one or more alkyl (including cycloalkyl) groups, alkoxycarbonyl (including cycloalkoxycarbonyl) groups, carboxylic acid groups, and/or nonaromatic heterocyclic amine groups; and
where the compound excludes (R)-Crizotinib or (S)-Crizotinib.

In an example embodiment, the aryl alkoxy group of R² is a hydrogen group, a C₄-C₆ alkyl ether group, a benzyloxy group, a 2-(phenyl)ethoxy group, or a chiral 1-(phenyl)ethoxy group, and wherein R² is optionally independently substituted at one or more aryl ring positions with one or more fluorine groups and/or one or more chlorine groups. In an example embodiment, the aryl alkoxy group of R² is a 1-(2,6-dichloro-3-fluorophenylethoxy) group comprising an (R) configuration, a 2-(2,6-dichloro-phenylethoxy) group, or a (2,6-dichloro-3-fluorobenzyloxy) group.

In an example embodiment, the aromatic heterocyclic amine group of R³ is a pyrazole group, a pyrrole group, or a pyrimidine group. In an example embodiment, the nonaromatic heterocyclic amine group of R³ is substituted at one or more positions with one or more piperidine groups optionally independently substituted at one or more positions with one or more formyl groups, alkyl (including cycloalkyl) groups, alkylalkynyl (including cycloalkylalkynyl) groups, and/or alkoxycarbonyl (including cycloalkycarbonyl) groups. In an example embodiment, the aromatic heterocyclic amine group R³ is a 1-(piperidin-4-yl)pyrazol-4-yl group, a 1-(N-alkylpiperidin-4-yl)pyrazol-4-yl group, or a 1-(N-alkylalkynypiperidin-4-yl)pyrazol-4-yl group.

In an example embodiment, the compound is selected from the group consisting of:

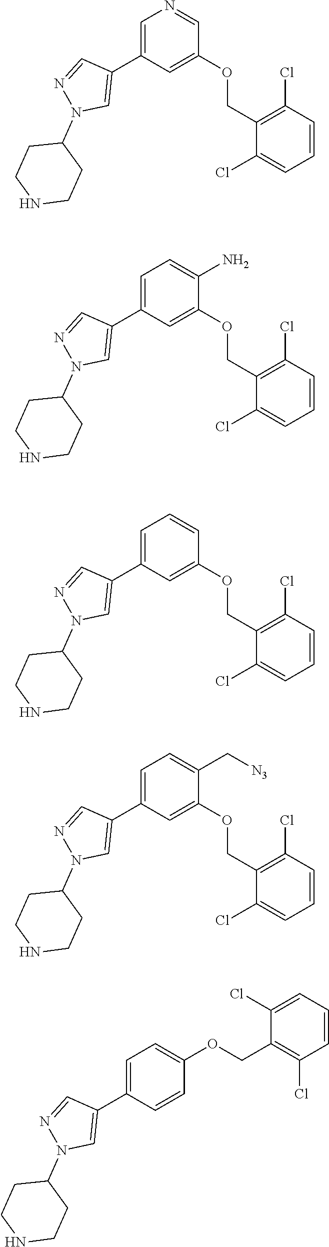 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

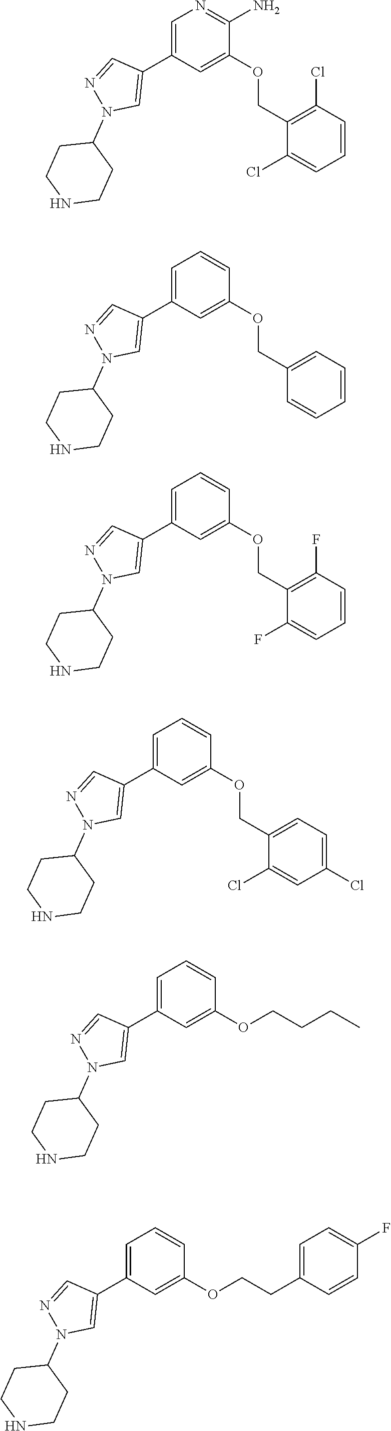 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

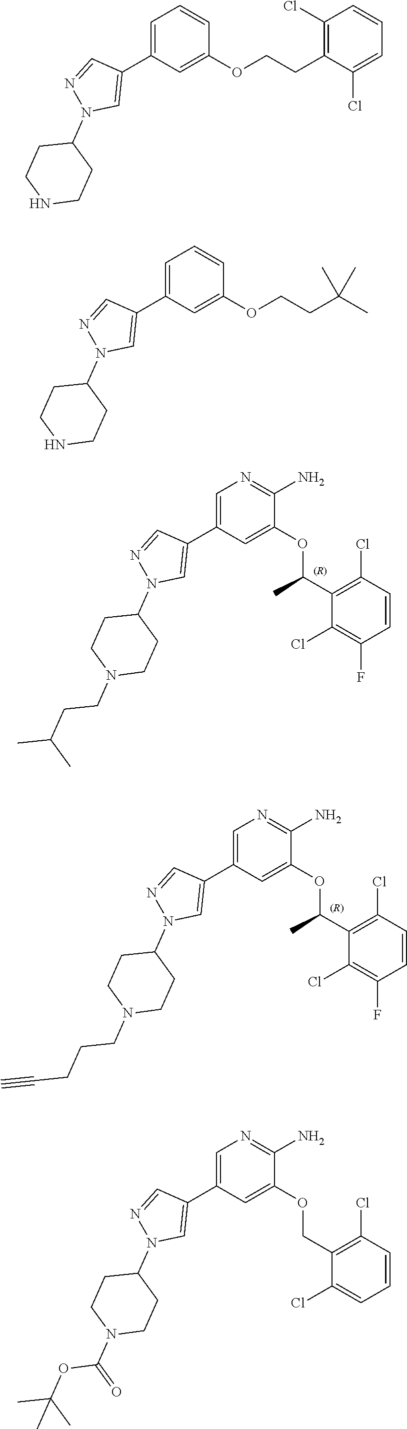

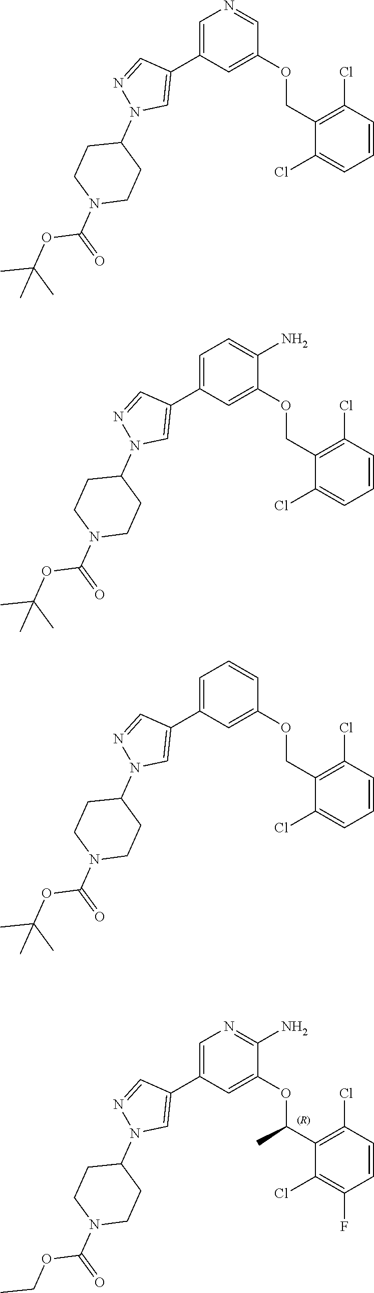

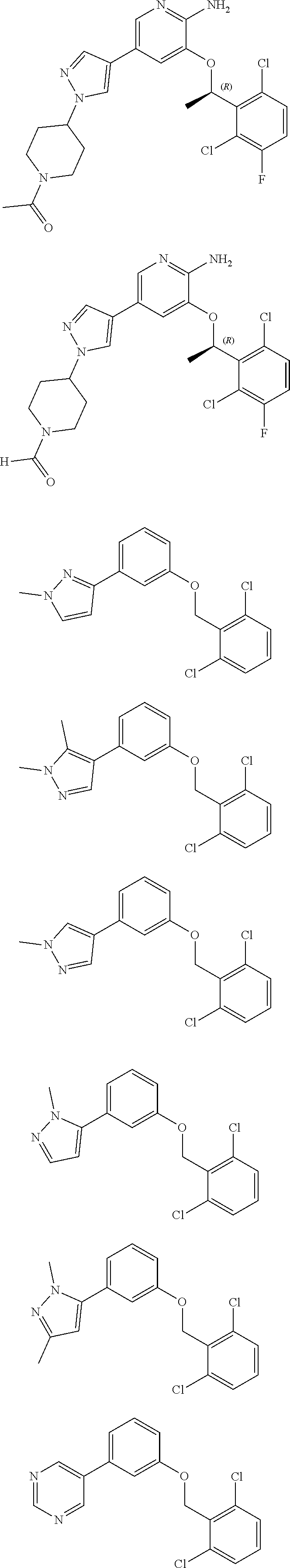

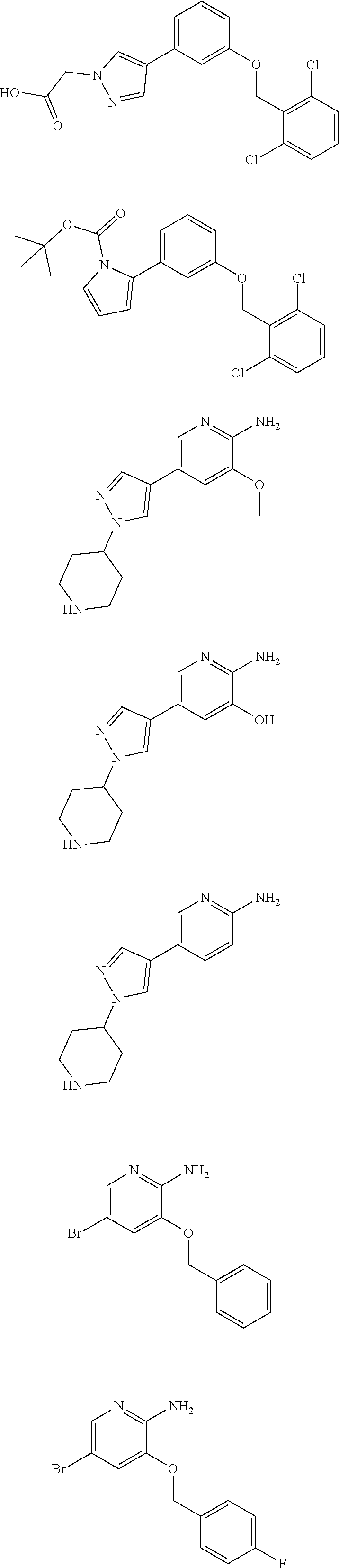

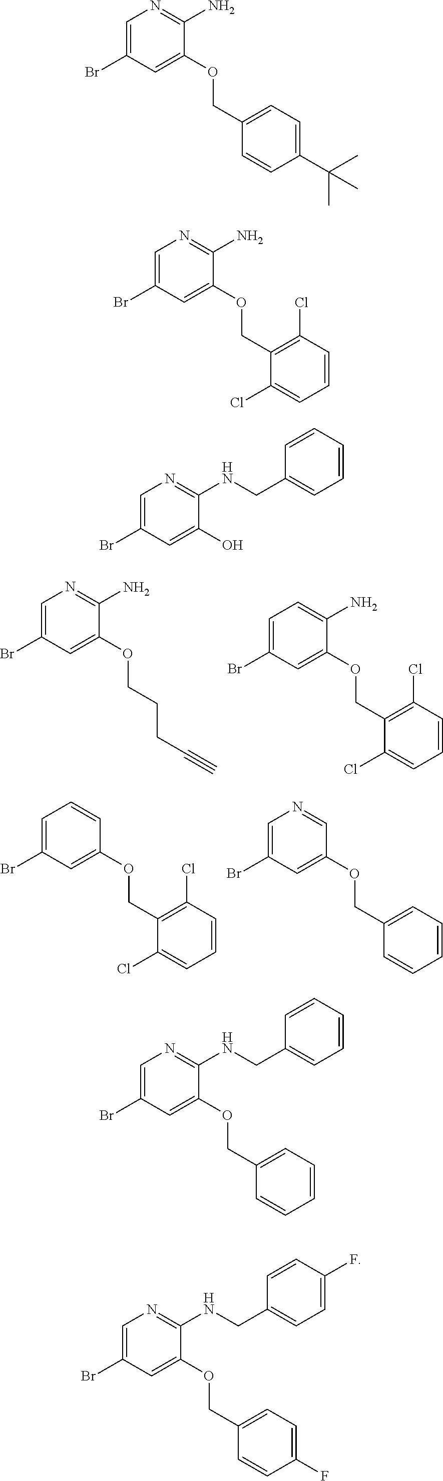

In an example embodiment, the compound is selected from the group consisting of:

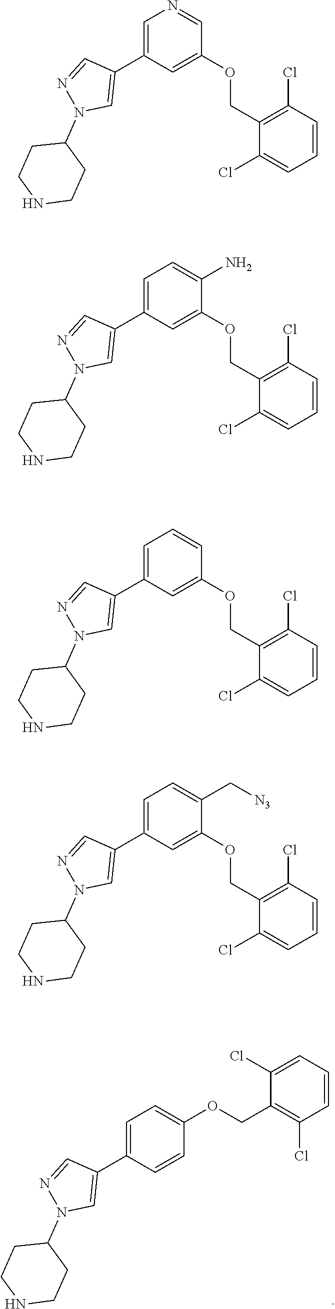 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

In an example embodiment, the compound is selected from the group consisting of:

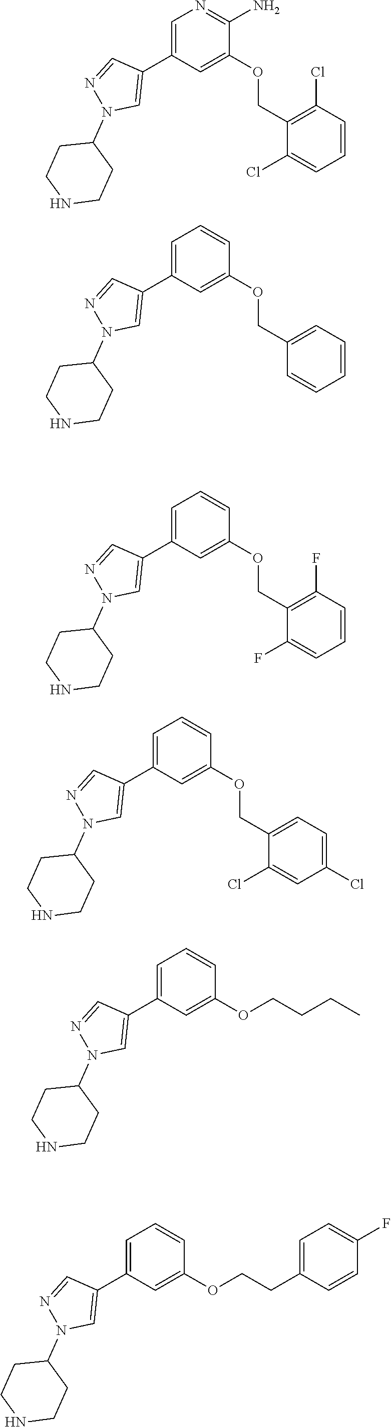 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

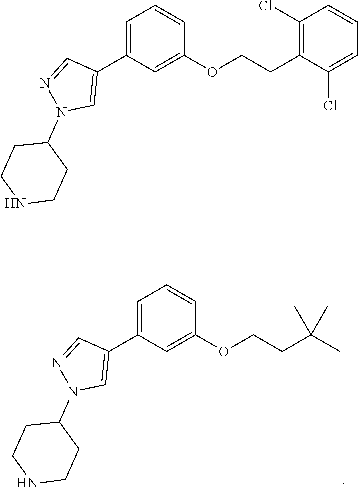 [SMILES: Clc1cccc(Cl)c1CCOc1cccc(-c2cnn(C3CCNCC3)c2)c1 ; CC(C)(C)CCOc1cccc(-c2cnn(C3CCNCC3)c2)c1]

In an example embodiment, the compound is selected from the group consisting of:

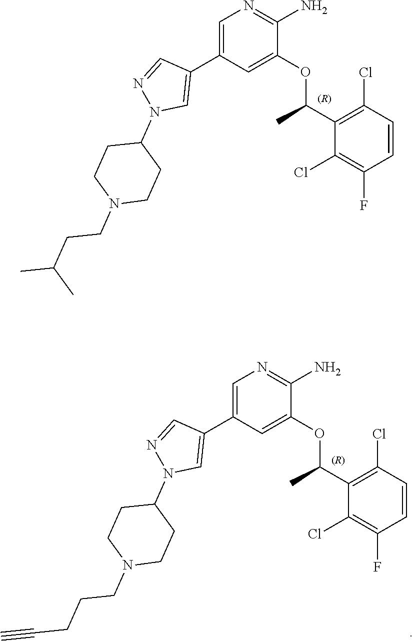

In an example embodiment, the compound is selected from the group consisting of:

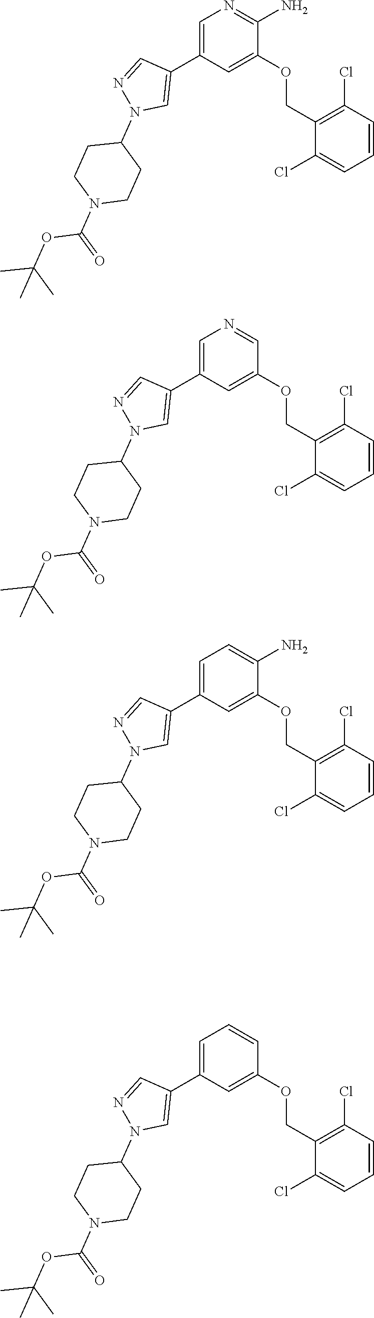 [SMILES: CC(C)(C)OC(=O)N1CCC(n2cc(-c3cnc(N)c(OCc4c(Cl)cccc4Cl)c3)cn2)CC1 ; CC(C)(C)OC(=O)N1CCC(n2cc(-c3cncc(OCc4c(Cl)cccc4Cl)c3)cn2)CC1 ; CC(C)(C)OC(=O)N1CCC(n2cc(-c3ccc(N)c(OCc4c(Cl)cccc4Cl)c3)cn2)CC1 ; CC(C)(C)OC(=O)N1CCC(n2cc(-c3cccc(OCc4c(Cl)cccc4Cl)c3)cn2)CC1]

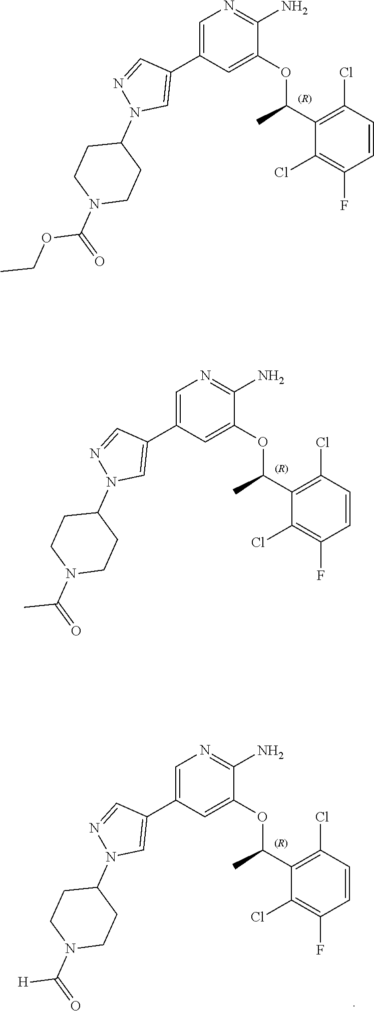

In an example embodiment, the compound is selected from the group consisting of:

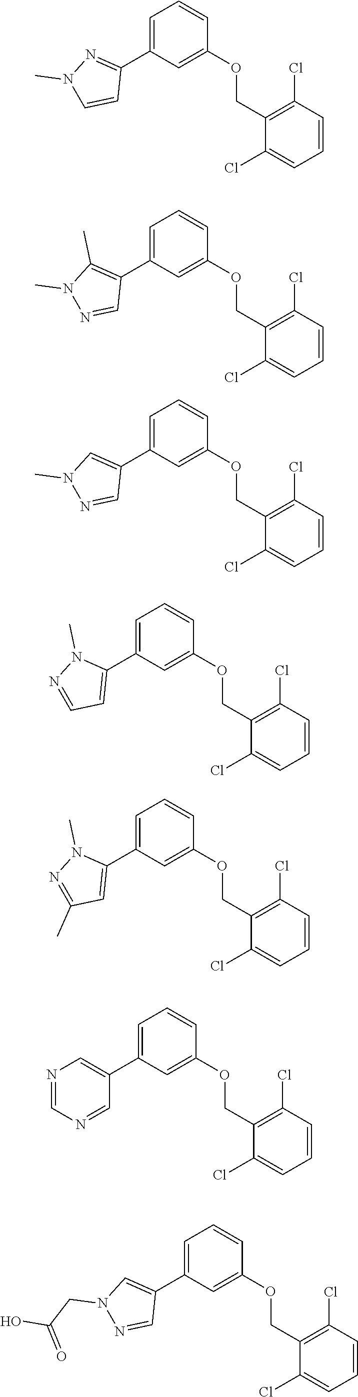

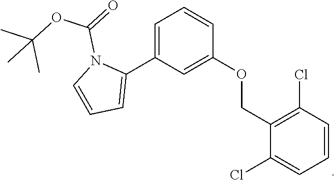 [SMILES: CC(C)(C)OC(=O)n1cccc1-c1cccc(OCc2c(Cl)cccc2Cl)c1]

In an example embodiment, the compound is selected from the group consisting of:

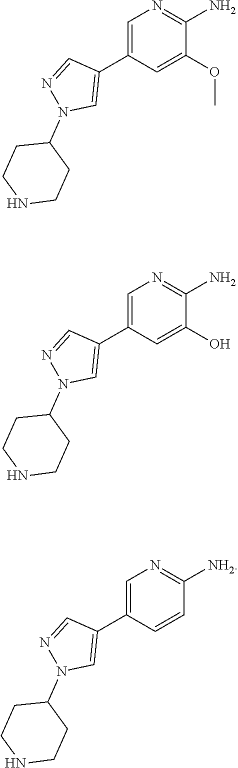 [SMILES: COc1cc(-c2cnn(C3CCNCC3)c2)cnc1N ; Nc1ncc(-c2cnn(C3CCNCC3)c2)cc1O ; Nc1ccc(-c2cnn(C3CCNCC3)c2)cn1]

In an example embodiment, the compound is selected from the group consisting of:

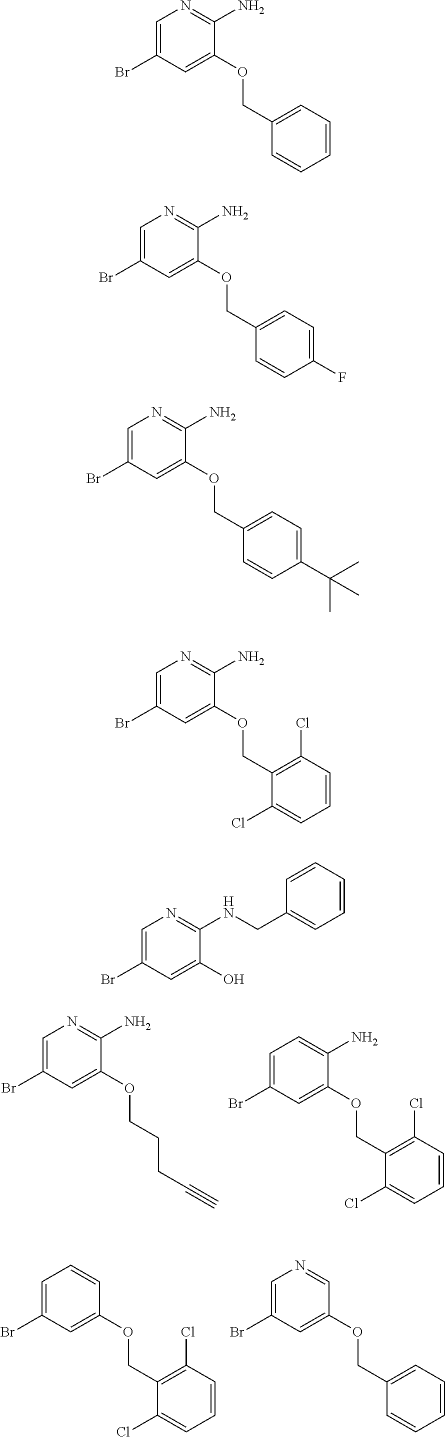

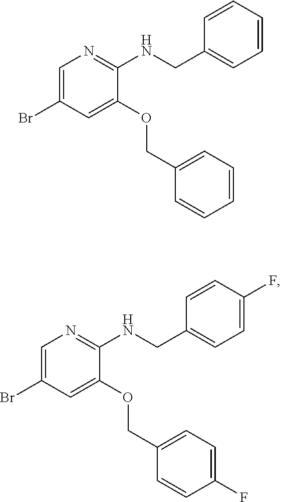 [SMILES: Brc1cnc(NCc2ccccc2)c(OCc2ccccc2)c1 ; Fc1ccc(CNc2ncc(Br)cc2OCc2ccc(F)cc2)cc1]

In one aspect, the present invention provides pharmaceutical formulations. In an example embodiment, a pharmaceutical formulation comprises one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In an example embodiment, the one or more dehydrocholesterol reductases comprise DHCR7, DHCR14, and/or DHCR24. In an example embodiment, a pharmaceutical formulation comprises one or more compound(s) of the present disclosure, (R)-Crizotinib, (S)-Crizotinib, AY 9944, analogs thereof, pharmaceutically acceptable salts thereof, or any pharmaceutical combination thereof.

In one aspect, the present invention provides a method for treating or preventing a bacterial infection. In an example embodiment, the method comprises: administering to a subject in need thereof a therapeutically effective amount of a compound of the present disclosure (R)-Crizotinib, (S)-Crizotinib, AY 9944, analogs thereof, pharmaceutically acceptable salts thereof, or any pharmaceutical combination thereof.

In an example embodiment, the bacterial infection is an acute bacterial infection, a chronic bacterial infection, a latent bacterial infection, a slow bacterial infection, a persistent bacterial infection, or any combination thereof. In an example embodiment, the bacterial infection is caused by an antibiotic resistant bacteria, a dormant bacteria, a bacteria present in a biofilm, or any combination thereof. In an example embodiment, the bacterial infection is caused by a Gram-negative bacteria or a Gram-positive bacteria.

In an example embodiment, the bacterial infection is a central nervous system infection, an eye infection, an ear infection, an upper respiratory tract infection, a lower respiratory tract infection, gastrointestinal infection, a heart infection, a gallbladder infection, a urinary tract infection, a skin infection, a blood infection, a bone infection, a hospital-acquired infection, a wound infection, a vaginal infection, a sexually transmitted disease, or any combination thereof.

In an example embodiment, the lower respiratory infection is caused by M. tuberculosis, S. aureus, P. aeruginosa, K. pneumoniae, L. pneumophila, S. pneumoniae, B. cenocepacia, H. influenzae, M. catarrhalis, M. pneumoniae, M. abscessus, M. avium, or any combination thereof.

In an example embodiment, the heart infection is caused by S. aureus, other Staphylococcus spp, S. gallolyticus, other Streptococcus spp, Enterococcus spp, Haemophilus spp, Aggregatibacter spp, Cardiobacterium spp, Eikenella spp, Kingella spp, or any combination thereof.

In an example embodiment, the gallbladder infection is caused by S. Typhi.

In an example embodiment, the gastrointestinal infection is caused by H. pylori, S. Enteritidis, S. Typhimurium, S. Typhi, S. aureus, Shigella sp., E. coli, V. cholerae, C. jejuni, Clostridium sp., B. cereus, Yersinia sp., or any combination thereof.

In an example embodiment, the urinary tract infection is caused by uropathogenic Escherichia coli (UPEC), K. pneumoniae, P. aeruginosa, S. saprophytis, S. aureus, P. mirabilis, S. marcescens, Enterobacter spp, or any combination thereof.

In an example embodiment, the bone infection is caused by S. aureus, other Staphylococcus spp, H. influenzae, Streptococcus spp, Pseudomonas spp, Enterobacter spp, or any combination thereof.

In an example embodiment, the wound infection is caused by S. aureus, S. pneumonia, E. coli, P. aeruginosa, Proteus mirablis, S. epidermis, Cornynebacterium spp, Klebsiella spp, other Staphylococcus spp, other Streptococcus spp, Enterococcus spp, or any combination thereof.

In an example embodiment, the bacterial infection is a persistent bacterial infection. In an example embodiment, the persistent bacterial infection is a persistent lung infection, a persistent heart infection, a persistent gall bladder infection, a persistent bone infection, a persistent wound infection, or a persistent urinary tract infection, or any combination thereof. In an example embodiment, the persistent bacterial infection is caused by a Gram-negative bacteria.

In an example embodiment, the persistent bacterial infection is a persistent urinary tract infection. In an example embodiment, the persistent urinary tract infection is caused by UPEC, K. pneumoniae, or P. aeruginosa, or any combination thereof.

In one aspect, the present invention provides a method for inducing antimicrobial activity against a bacterial infection. In an example embodiment, the method comprises: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In an example embodiment, the method comprises: administering to a subject in need thereof a therapeutically effective amount of one or more compounds of the present disclosure, (R)-Crizotinib, (S)-Crizotinib, AY 9944, analogs thereof, pharmaceutically acceptable salts thereof, or any pharmaceutical combination thereof.

In an example embodiment, the antimicrobial activity is induced in host cells of the subject infected with a bacteria. In an example embodiment, the bacterial infection is an acute bacterial infection, a chronic bacterial infection, a latent bacterial infection, a slow bacterial infection, a persistent bacterial infection, or any combination thereof. In an example embodiment, the bacterial infection is caused by an antimicrobial resistant bacteria, a dormant bacteria, a bacteria present in a biofilm, or any combination thereof. In an example embodiment, the bacterial infection is caused by a Gram-negative bacteria or a Gram-positive bacteria. In an example embodiment, the bacterial infection is a central nervous system infection, an eye infection, an ear infection, an upper respiratory tract infection, a lower respiratory tract infection, gastrointestinal infection, a heart infection, a gallbladder infection, a urinary tract infection, a skin infection, a blood infection, a bone infection, a hospital-acquired infection, a wound infection, a vaginal infection, a sexually transmitted disease, or any combination thereof.

In an example embodiment, the lower respiratory infection is caused by M. tuberculosis., S. aureus, P. aeruginosa, K. pneumoniae, L. pneumophila, S. pneumoniae, B. cenocepacia, H. influenzae, M. catarrhalis, M. pneumoniae, M. abscessus, M. avium, or any combination thereof.

In an example embodiment, the heart infection is caused by S. aureus, other Staphylococcus spp, S. gallolyticus, other Streptococcus spp, Enterococcus spp, Haemophilus spp, Aggregatibacter spp, Cardiobacterium spp, Eikenella spp, Kingella spp, or any combination thereof.

In an example embodiment, the gallbladder infection is caused by S. Typhi.

In an example embodiment, the gastrointestinal infection is caused by H. pylori, S. Enteritidis, S. Typhimurium, S. Typhi, S. aureus, Shigella sp., E. coli, V. cholerae, C. jejuni, Clostridium sp., B. cereus, Yersinia sp., or any combination thereof.

In an example embodiment, the urinary tract infection is caused by uropathogenic Escherichia coli (UPEC), K. pneumoniae, P. aeruginosa, S. saprophytis, S. aureus, P. mirabilis, S. marcescens, Enterobacter spp, or any combination thereof.

In an example embodiment, the bone infection is caused by S. aureus, other Staphylococcus spp, H. influenzae, Streptococcus spp, Pseudomonas spp, Enterobacter spp, or any combination thereof.

In an example embodiment, the wound infection is caused by S. aureus, S. pneumonia, E. coli, P. aeruginosa, Proteus mirablis, S. epidermis, Cornynebacterium spp, Klebsiella spp, other Staphylococcus spp, other Streptococcus spp, Enterococcus spp, or any combination thereof.

In an example embodiment, the bacterial infection is a persistent bacterial infection. In an example embodiment, the persistent bacterial infection is a persistent lung infection, a persistent heart infection, a persistent gall bladder infection, a persistent bone infection, a persistent wound infection, or a persistent urinary tract infection, or any combination thereof. In an example embodiment, the persistent bacterial infection is caused by a Gram-negative bacteria. In an example embodiment, the persistent bacterial infection is a persistent urinary tract infection. In an example embodiment, the persistent urinary tract infection is caused by UPEC, K. pneumoniae, or P. aeruginosa, or any combination thereof.

In an aspect, the present invention provides a method of treating or preventing a viral infection. In an example embodiment, the method comprises: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In an example embodiment, the method comprises: administering to a subject in need thereof a therapeutically effective amount of one or more compounds of the present disclosure, (R)-Crizotinib, (S)-Crizotinib, AY 9944, analogs thereof, pharmaceutically acceptable salts thereof, or any pharmaceutical combination thereof.

In an example embodiment, the viral infection is an acute viral infection, a chronic viral infection, a latent viral infection, a slow viral infection, a persistent viral infection, or any combination thereof. In an example embodiment, the viral infection is caused by an antimicrobial resistant virus and/or a dormant virus.

In an example embodiment, the viral infection is caused by a Coronaviridae virus. In an example embodiment, the Coronaviridae virus is a from β-coronavirus. In an example embodiment, the β-coronavirus is SARS-COV-2.

In an aspect, the present invention provides a method of inducing antimicrobial activity against a viral infection. In an example embodiment, the method comprises: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In an example embodiment, the method comprises: administering to a subject in need thereof a therapeutically effective amount of one or more compounds of the present disclosure, (R)-Crizotinib, (S)-Crizotinib, AY 9944, analogs thereof, pharmaceutically acceptable salts thereof, or any pharmaceutical combination thereof.

In an example embodiment, the antimicrobial activity is induced in host cells of the subject infected with a virus. In an example embodiment, the viral infection is an acute viral infection, a chronic viral infection, a latent viral infection, a slow viral infection, a persistent viral infection, or any combination thereof. In an example embodiment, the viral infection is caused by an antimicrobial resistant virus and/or a dormant virus.

In an example embodiment, the viral infection is cause by a Coronaviridae virus. In an example embodiment, the Coronaviridae virus is a from β-coronavirus. In an example embodiment, the β-coronavirus is SARS-COV-2.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, in conjunction with the accompanying drawings described herein.

FIGS. 1A-1E. The intracellular environment of bladder epithelial cells enhances antibiotic tolerance of Klebsiella pneumoniae. (FIG. 1A) The clinical K. pneumoniae urine isolate RB120 persists intracellularly in human 5637 bladder epithelial cells over the course of 48 hours. A gentamicin protection assay was used to kill and prevent growth of extracellular bacteria. Mean and SD of colony forming units (CFUs) recovered from plated cell lysates from n=3 independent experiments shown. (FIG. 1B) K. pneumoniae RB120 persists in LAMP1 positive vacuoles. Representative confocal microscopy image of bladder epithelial cells infected with mNeon expressing K. pneumoniae and immunolabeled with LAMPI antibodies at 48 hours post infection. Mean and SD of LAMP1 positive Klebsiella-containing vacuoles from n=2 independent experiments is 83.2%±7.1%. Scale bars=10 μM. (FIG. 1C) The replication-dependent carbapenem antibiotic meropenem is ineffective against intracellular K. pneumoniae at 10 μg/ml (150× of the minimal inhibitory concentration determined in axenic medium) over the course of 20 hours. Treatment of Salmonella Typhimurium 14028 (10 μg/ml meropenem, 150× minimal inhibitory concentration) was used as a positive control. Antibiotic treatment was initiated 4 hours post infection (hpi) with infected host cells treated with either gentamicin (gent) or meropenem (mero). Mean and SD of n=3 independent experiments shown. (FIG. 1D) Intracellular survival and antibiotic persistence of K. pneumoniae results in delayed outgrowth after host cell lysis and incubation in axenic growth medium (LB). Similar numbers of intracellular bacteria were liberated from host cells via cell lysis at indicated timepoints and incubated in LB medium to enable outgrowth and plating for CFUs at indicated timepoints. Ciprofloxacin (cipro) persisters were treated for 24 h with ciprofloxacin at 24 hours post infection (see FIG. 1E). Mean and SD of n=3 biological replicates shown. (FIG. 1E) Ciprofloxacin is active against non-replicating bacteria. Ciprofloxacin treatment results in biphasic killing of K. pneumoniae, revealing antibiotic persister subpopulations occurring at substantially increased frequency in bladder epithelial cells compared to axenic medium (LB) or phosphate-buffered saline (PBS). Treatment was initiated with 4 ug/ml ciprofloxacin (100× of the MIC in axenic medium) 24 hours post infection. Mean and SD of n=3 (intracellular) or n=2 (LB) independent experiments shown.

FIGS. 2A-2D. Crizotinib enantiomers emerge as top hits in a high-throughput screening assay of repurposed clinical compounds against intracellular non-replicating antibiotic-tolerant K. pneumoniae. (FIG. 2A) Overview of a screening assay for impaired intracellular survival. Bladder epithelial cells were infected with lux-expressing K. pneumoniae and incubated with the carbapenem antibiotic meropenem (see FIG. 1A), after killing extracellular bacteria with a high dose of gentamicin shortly after the infection. Intracellular survival was assessed by determining the luminescence intensity of intracellular bacteria that had been liberated by host cell lysis and outgrown in LB medium for 7 hours. The screening assay was validated with 4 batches of 8 96-well plates treated with ciprofloxacin-containing medium (positive control) or DMSO-containing medium (negative control), added in checkerboard format and to entire plates in alternating fashion. Mean and SD of Z-factors of n=32 plates from 4 batches (independent outgrowth events) shown. (FIG. 2B) Correlation of different concentrations of ciprofloxacin (positive control) with luminescence levels and recovered CFUs. Mean and SD of luminescence levels from n=2 independent experiments with 5 96-well plates shown (circles, right Y-axis) (R²=0.8756), as well as mean and SD of the percentage of CFUs (squares, left Y-axis) recovered from n=3 independent experiments and immediately plated after liberation (cell lysis) relative to gentamicin treated infections shown. (FIG. 2C) Overview of primary and secondary screen (retesting) outcome. 3975 compounds were screened in a primary screen in duplicates at a concentration of 5 μM. Partial hits and hits involving known antibiotics were excluded. Hits from the primary screen were retested in a secondary screen at different concentration. (FIG. 2D) Structure and documented targets of the anticancer compounds (R)-and (S)-crizotinib. (R)-crizotinib is a receptor tyrosinase kinase inhibitor (ALK, HGFR, c-Met) approved by the FDA for treatment of small lung cell cancer with ALK rearrangements. (S)-crizotinib is a MTH1 inhibitor in preclinical trials for anticancer treatment. MTHI sanitizes oxidized nucleotides that occur in fast growing cancer cells (prevents lethal mutations caused by reactive oxygen species (ROS)).

FIGS. 3A-3J. Crizotinib induces rapid intracellular antibacterial and antiviral activity. (FIG. 3A) (R)-and (S)-crizotinib display dose-dependent antibacterial activity against intracellular K. pneumoniae. Bladder epithelial cells were infected with K. pneumoniae (RB120) and treated with (R)- or (S)-crizotinib for 24 hours (from 24 to 48 hours post infection) in the presence of gentamicin to prevent extracellular bacterial growth. Mean and SD of the percentage of recovered bacteria (from plated cell lysates) relative to untreated DMSO controls (t=48 hpi) from n=3 independent experiments with 96 well plates shown. (FIG. 3B) (R)-crizotinib displays dose-dependent host cell toxicity after 24 h treatment of infected bladder cells. Host cell toxicity was determined with CellTiter-Glo, which assesses ATP levels in cell lysates via the ATP-dependent luciferase reaction. Mean and SD of the percentage of viability of treated cells relative to untreated DMSO controls (t=48 hpi) from n=3 independent experiments with 96well plates shown. (FIG. 3C) (S)-crizotinib displays rapid intracellular antiinfective activity against intracellular Klebsiella pneumoniae. Infected bladder epithelial cells were treated with 10 μM (S)-crizotinib over 2 hours. Mean and SD of the percentage of recovered bacteria relative to untreated DMSO control (t=2 h) from n=2 independent experiments shown (80% intracellular killing in 15 minutes). (FIG. 3D) (S)-crizotinib displays antiinfective activity against intracellular ciprofloxacin-tolerant K. pneumoniae persisters. Infected bladder cells were treated with 4 mg/ml ciprofloxacin for 14 hours (from 10 to 24 hours post infection) followed by treatment with (S)-crizotinib for 6 hours, in the presence of ciprofloxacin. Mean and SD of the percentage of recovered bacteria relative to DMSO controls (t=30 hpi) from n=2 independent experiments with 96 well plates shown. (FIG. 3E) (S)-crizotinib induces antimicrobial activity in host cells. (S)-crizotinib ((S)-crizo) displays antiinfective properties against infected bladder epithelial cells in the absence of gentamicin (gent). Bladder cells were infected with K. pneumoniae and subjected to a gentamicin protection assay to eliminate extracellular bacteria and treated at 24 hours post infection for 2 hours with 10 μM (S)-crizotinib in the presence and absence of gentamicin. Mean and SD of the percentage of recovered bacteria from plated cell lysates relative to DMSO controls from n=3 independent experiments with 24 well plates shown. (FIG. 3F) (S)-crizotinib does not display antimicrobial activity against K. pneumoniae in axenic medium (LB medium). Growth of K. pneumoniae in LB medium with indicated concentrations of (S)-crizotinib was determined by measuring optical density after 24 hours of treatment. Percentage of growth relative to untreated K. pneumoniae from a representative experiment with n=4 biological replicates shown. (FIG. 3G) (S)-crizotinib reduces overall number (by 63%) and fluorescence intensity (by 33%) of fluorescent Klebsiella-containing vacuoles. Bladder epithelial cells were infected with capsule-deficient K. pneumoniae ST258 expressing the fluorescent protein mNeon (UCI_38DwbaP_mNeon) and treated for 24hours with 10 μM (S)-crizotinib from 24 to 48 hours post infection. The strain was previously characterized and shown to have a high tendency to grow in LAMP1-positive compartments, resulting in the frequent formation of fluorescent pods. Klebsiella-containing compartments were identified via immunofluorescence straining of LAMP1 followed by confocal microscopy and automatic detection via image analysis software under identical conditions. A representative image displaying Klebsiella-containing compartments with weaker fluorescence and distorted pods, as well as global quantification of the fluorescence intensity of detectable mNeon-expressing Klebsiella in LAMP1 positive compartments is shown. 2197 (untreated) vs 2344 (treated) exposed host cells analyzed. Median and interquartile range of n=122 untreated vs n=45 treated Klebsiella-containing compartments from 2 independent experiments shown. Significance calculated with a nonparametric t-test. (FIG. 3H) (S)-crizotinib displays antiinfective activity against other major uropathogens and Salmonella Typhimurium. Bladder epithelial cells were infected with carbapenem-resistant K. pneumoniae (sequence type ST258, UCI_38DwbaP), uropathogenic Escherichia coli (UPEC, UTI89) or Pseudomonas aeruginosa (PAO1), and treated for 4 hours (ST258, UPEC) or 24 hours (P. aeruginosa) with 10 μM (S)-crizotinib (treatment initiated at 24 hours post infection). HeLa epithelial cells were infected with S. Typhimurium and treated for 20 hours (4 to 24 hours post infection). Mean and SD of the percentage of recovered bacteria from plated cell lysates relative to DMSO treated cells from n=3 independent experiments in 24 well plates shown. (FIG. 3I) (S)-crizotinib displays rapid antiinfective activity against intracellular K. pneumoniae in the human lung epithelial cell line A549. Infected lung epithelial cells were treated with 10 μM (S)-crizotinib over 2 hours. Mean and SD of the percentage of recovered bacteria relative to untreated DMSO control (t=2 h) from n=2 independent experiments with 96 well plates shown. (FIG. 3J) (S)-crizotinib displays antiviral activity against kidney epithelial cells (veroE6) infected with SARS-COV-2. VeroE6 epithelial cells were exposed for 1 hour to SARS-COV-2 at an MOI of 0.1 and treated for 24 h with (S)-crizotinib. Immunofluorescence microscopy with antibodies directed against SARS-CoV-2 enabled the detection and quantification via image analysis software. Mean and SD of normalized percentage of detected virus vs untreated DMSO control from n=3 biological replicates from one representative experiment shown.

FIGS. 4A-4B. MTHI inhibitors TH287 and TH588 induce moderate intracellular bacterial activity and dose dependent toxicity against intracellular K. pneumoniae. Infected bladder epithelial cells were treated with 10 μM MTH1 inhibiting compounds for 24 hours (FIG. 4A, 24-48 hours post infection) or 4 hours (FIG. 4B, 24-28 hours post infection). Mean and SD of the percentage of recovered bacteria, percentage of bladder cell viability (determined with ATP-dependent CellTiter-Glo) and percentage of extracellular growth in axenic medium, relative to untreated DMSO controls from n=2 independent experiments with 96 well plates (FIG. 4A: bladder cell viability; FIG. 4B: intracellular survival) shown.

FIGS. 5. (S)-crizotinib treatment does not result in exocytosis of Klebsiella-containing vacuoles. Bladder epithelial cells were infected with K. pneumoniae (RB120) and treated with (S)-crizotinib for 2 hours (from 24 to 26 hours post infection) in the absence of gentamicin to prevent extracellular killing. Mean and SD of the percentage of recovered bacteria (from supernatants (“SN”) and plated cell lysates) relative to untreated DMSO controls (t=26 hpi) from n=3 independent experiments with 24 well plates shown.

FIGS. 6A-6B. (S)-crizotinib displays efficacy in a mouse model of latent urinary tract infection with K. pneumoniae. (FIG. 6A) Overview of a mouse model of latent infection used to demonstrate in vivo efficacy of (S)-crizotinib. Female Balb/c mice were transurethrally infected with the uropersistent K. pneumoniae ST258 strain UCI_38ΔwbaP and treated subcutaneously for 3 consecutive days with 25 mg/kg (S)-crizotinib 3 days post infection. Bladders were harvested 3 days post treatment and immediately lysed and plated to determine bacterial titers. (FIG. 6B) (S)-crizotinib reduces bacterial bladder burden in a mouse model of latent infection. n=25 mice infected per group. Significance calculated with a Mann-Whitney U test. Limit of detection (5 bacteria) indicated with a dashed line.

FIG. 7. (S)-crizotinib and AY 9944 do not induce an interferon beta response in bladder epithelial cells infected with K. pneumoniae. Infected cells were treated for 24 hours with 10 mM (S)-crizotinib, AY9944 or DMSO control from 24 to 48 hours post infection.

FIGS. 8A-8B. Structure-activity relationship (SAR) Analysis. Intracellular survival of Klebsiella pneumoniae (RB120) after four-hour treatment with 10 μM crizotinib analogs predicted structures. Mean percentage of recovered bacteria from treated bladder epithelial cells relative to untreated DMSO control from n=3 biological replicates in 96 well plates shown. (FIG. 8A) Exemplary structures shown have similar or equivalent antimicrobial activity as (R)-crizotinib and (S)-crizotinib. Comparison of (S)-crizotinib with the boxed structure shows that chirality, central amines (including primary amine), chlorine, and fluorine are dispensable for antimicrobial activity. (FIG. 8B) Exemplary structures display substantially reduced antibacterial activity compared to (R)-crizotinib and (S)-crizotinib.

FIG. 9. Specific inhibition of MTH1 was recently observed with an (S)-crizotinib affinity probe.

FIG. 10. A thermal-shift stability assay with whole cells recently identified additional targets of (S)-crizotinib. MTH1 (a documented target of (S)-crizotinimb; Nuclear Export Protein; Tubulin Finding Cofactor D; DNA polymerase alpha catalytic subunit (DNA repair); CCCH-Type Antiviral Protein 1 (RNA degradation); Plakophilin-2 (links cadherins to intermediate filaments); and Dehydrocholesterol reductase (final step in cholesterol biosynthesis).

FIG. 11. 7-Dehydrocholesterol reductase identified as potential target of (S)-crizotinib. The 7-dehydrocholesterol reductase inhibitor AY9944 also a hit in the screen.

FIG. 12. The 7-dehydrocholesterol reductase inhibitor AY9944 mirrors (S)-crizotinib antimicrobial activity. AY9944 was observed to induce dose-dependent non-toxic antimicrobial activity (measured at 24 hours), rapid antimicrobial activity (measured at 10 μM AY9944), and antiviral activity against SARS-COV-2.

FIG. 13. AY9944 also binds to other enzymes of post-lanosterol pathway at micromolar concentrations.

FIG. 14. Other 7-dehyrocholesterol reductase (DHCR7) and EBP inhibitors do not induce dose-dependent antimicrobial activity. Inhibition of DHCR7, EBP unlikely to induce antimicrobial activity, and DHCR7, EBP unlikely to be the antimicrobial target of (S)-crizotinib.

FIGS. 15A-15B. (FIG. 15A) Modest dose-dependent antimicrobial activity of MTH1 inhibitor TH588 after 24 hours of treatment. (FIG. 15B) MTH1 inhibitor TH588 did not induce intracellular antibacterial activity after 4 hours of treatment (10 μM).

FIG. 16. Modest antimicrobial activity of TLR7 agonist imiquimod after 24 hours of treatment.

FIGS. 17A-17C. LC-MS based lipidomics evaluation of effect of other available post-lanosterol cholesterol biosynthesis inhibitors on infection. (FIG. 17A) Post-lanosterol pathway. (FIG. 17B) Effect of 10 μM AY9944 or 10 μM (S)-crizotinib after 2 hours of treatment (FIG. 17C) or after 24 hours of treatment. (S)-crizotinib treatment is associated with accumulation of lanosterol and FF-MAS.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/±10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “antimicrobial resistant” microbe is used herein to refer to a microbe which has evolved mechanisms to protect against the effects of one or more antimicrobials. Microbes resistant to multiple antimicrobials are also referred to herein as “multi-drug resistant.” Treatment of an infection by an “antimicrobial resistant” microbe may require a higher dosage, a prolonged drug course, and/or repeated drug courses of the one or more antimicrobials to which the microbe has developed resistance or may require an alternative medication.

The term “symptomatic infection” is used herein to refer to an infection with clinically apparent symptoms.

The term “asymptomatic infection” is used herein to refer to an infection without clinically apparent symptoms. Upon future reactivation, an asymptomatic infection can become a symptomatic infection.

The term “acute infection” is used herein to refer to an infection which develops suddenly. An acute infection has an acute disease which may be symptomatic or asymptomatic.

The term “chronic infection” is used herein to refer to an infection which is continually present following a primary infection. A chronic infection may have chronic or recurrent disease which may be symptomatic or asymptomatic.

The term “latent infection” is used herein to refer to an infection by a microbe (“dormant microbe”) which lacks demonstrable infections microbes between episodes of a recurrent disease which may be symptomatic or asymptomatic. The term “dormant” microbe is used herein to refer to a microbe existing in a metabolically inactive (e.g., slow growing or nongrowing) state.

The term “slow infection” is used herein to refer to an infection by a microbe characterized by a prolonged incubation period followed by a progressive disease which may be symptomatic or asymptomatic.

The term “persistent infection” is used herein to refer to a microbe (“persister cell”) which is not effectively cleared (e.g., by host immune response) or sterilized (e.g., by antimicrobial treatment) from the host following primary infection. A persistent infection may continue over the course of weeks, months, or years. A persistent infection may be a chronic infection, a slow infection, and/or a latent infection and may be defined by the presence of persister cells, which may have increased treatment (e.g. antibiotic) tolerance or resistance. A persistent infection may be caused by an antimicrobial resistant microbe. A persistent infection may be a symptomatic infection or an asymptomatic infection. A persistent infection which is not effectively sterilized by a standard drug course may require a higher dosage, a prolonged drug course, and/or repeated drug courses. A symptomatic persistent infection may be suppressed by a higher dosage, a prolonged drug course, and/or repeated drug courses such that it becomes an asymptomatic persistent infection. A persistent infection may be an unresolved infection that exists over a course of weeks to months or may be defined based on a number of failed treatments or identification of antibiotic resistance.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification may all refer to the same embodiment but are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Since most antibiotic discovery efforts have relied on the identification of compounds that limit bacterial growth in axenic medium, Applicants pursued a different approach by specifically targeting intracellular bacteria and by screening for compounds that do not directly target bacteria but instead induce an antimicrobial response in infected host cells. Applicants focused on the Gram-negative pathogen Klebsiella pneumoniae, which is one of the most concerning antibiotic resistance threats and one of the most common causes of hospital-associated infections, and on persistent urinary tract infections, a common type of persistent infection. Applicants established that intracellular K. pneumoniae that persist in bladder epithelial cells display widespread antibiotic tolerance to carbapenem antibiotics, which are the antibiotics of last resort to treat multidrug infections, and showed that the intracellular environment increases the frequency of a dormant, multidrug-tolerant subpopulation. Applicants developed a high-throughput intracellular viability screen that led to the identification of the clinical anticancer compound crizotinib, which exhibited a so far unknown anti-infective property that induced rapid antimicrobial activity in bladder epithelial cells infected with antibiotic tolerant K. pneumoniae. While crizotinib induced moderate cytotoxicity, Applicants found that its (S)-enantiomer, (S)-crizotinib, did not display cytotoxicity and induced rapid broad spectrum antimicrobial activity in host cells, which included activity against the three most dominant Gram-negative bacterial uropathogens (UPEC, K. pneumoniae, P. aeruginosa), extremely dormant intracellular persisters and, strikingly, antiviral activity against the coronavirus SARS-COV-2. Applicants further showed that (S)-crizotinib was effective in a mouse model of latent urinary tract infection with K. pneumoniae, suggesting that it has potential to be repurposed as a broad-spectrum anti-infective for the treatment of persistent bacterial infections and viral infections.

### Compounds

In one aspect, the present invention provides compounds, pharmaceutical compositions, and for use in methods for treating or preventing a microbial infection or for inducing anti-microbial activity against a microbial infection. In an example embodiment, a compound or a salt thereof has the structural formula:

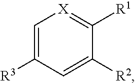

where X is C or N; wherein R¹ is a hydrogen group, an amino group, an alkyl (including cycloalkyl) amino group, an alkyl azide (including a cycloalkyl azide) group, an alkylaryl (including cycloalkylaryl) amino group, an alkylaryloxy (including cycloalkylaryloxy) group, or an aryl amino group, and where R¹ is optionally independently substituted at one or more aryl ring positions with one or more halogen groups; where R² is a hydrogen group, a hydroxyl group, an alkoxy (including cycloalkoxy) group, an alkynyl alkoxy (including alkynyl cycloalkoxy) group, or an aryl alkoxy (including aryl cycloalkoxy) group, and where, optionally, the aryl alkoxy (including aryl cycloalkoxy) group is independently substituted at one or more aryl ring positions with one or more halogen groups; wherein R³ is a halogen group or a ring carbon of an aromatic heterocyclic amine group, where, optionally, the aromatic heterocyclic amine group is independently substituted at one or more heterocyclic ring positions with one or more alkyl (including cycloalkyl) groups, alkoxycarbonyl (including cycloalkoxycarbonyl) groups, carboxylic acid groups, and/or nonaromatic heterocyclic amine groups; and where the compound excludes (R)-Crizotinib or (S)-Crizotinib.

In one example embodiment, the aryl alkoxy group of R² is a hydrogen group, a C⁴-C₆ alkyl ether group, a benzyloxy group, a 2-(phenyl)ethoxy group, or a chiral 1-(phenyl)ethoxy group, and where R² is optionally independently substituted at one or more aryl ring positions with one or more fluorine groups and/or one or more chlorine groups.

In one example embodiment, the aryl alkoxy group of R² is a 1-(2,6-dichloro-3-fluorophenylethoxy) group comprising an (R) configuration, a 2-(2,6-dichloro-phenylethoxy) group, or a (2,6-dichloro-3-fluorobenzyloxy) group.

In one example embodiment, the aromatic heterocyclic amine group of R³ is a pyrazole group, a pyrrole group, or a pyrimidine group.

In one example embodiment, the nonaromatic heterocyclic amine group of R³ is substituted at one or more positions with one or more piperidine groups optionally independently substituted at one or more positions with one or more formyl groups, alkyl (including cycloalkyl) groups, alkylalkynyl (including cycloalkylalkynyl) groups, and/or alkoxycarbonyl (including cycloalkycarbonyl) groups.

In one example embodiment, the aromatic heterocyclic amine group R³ is a 1-(piperidin-4-yl)pyrazol-4-yl group, a 1-(N-alkylpiperidin-4-yl)pyrazol-4-yl group, or a 1-(N-alkylalkynypiperidin-4-yl)pyrazol-4-yl group.

In one example, the compound is selected from the group consisting of:

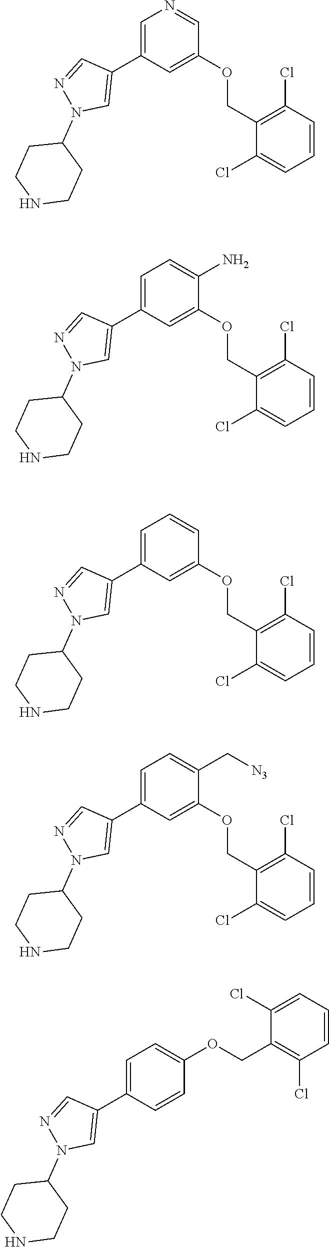 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

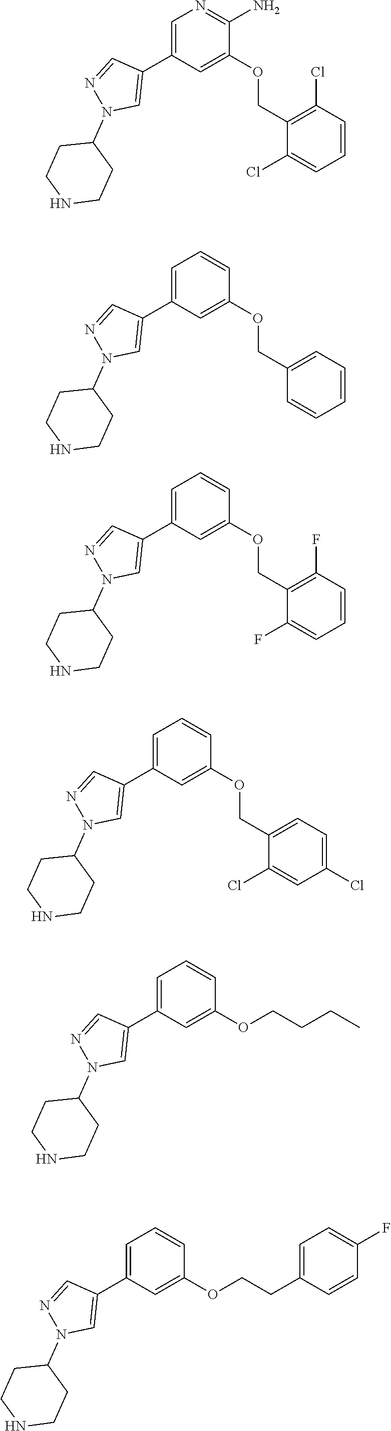 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

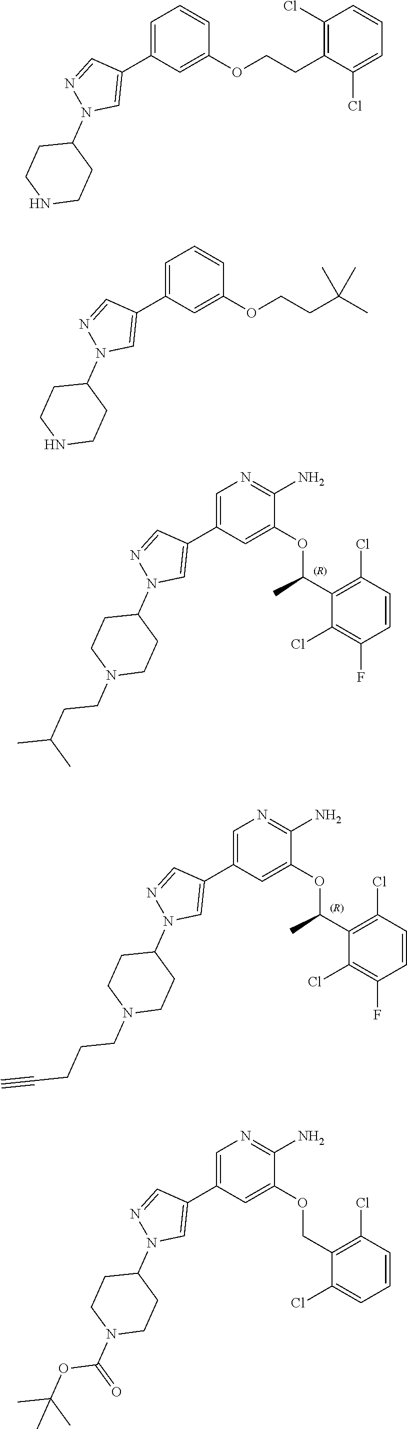

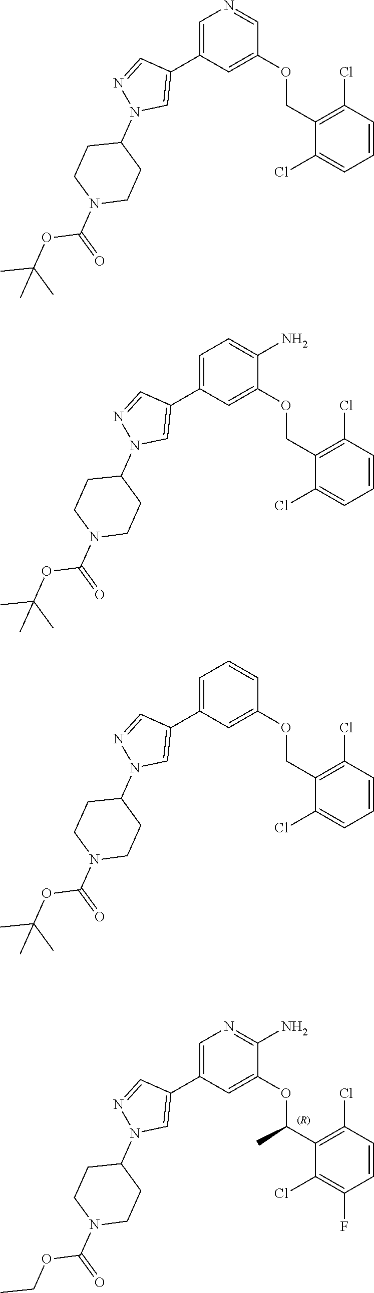

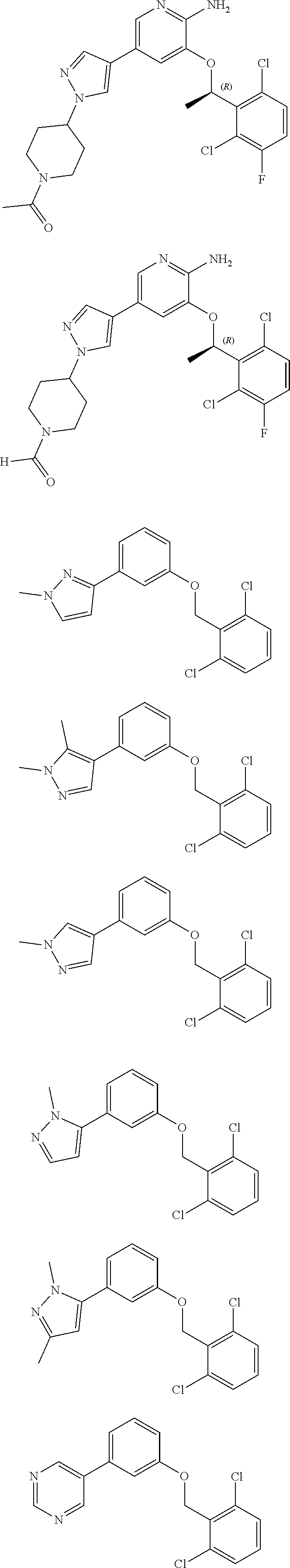

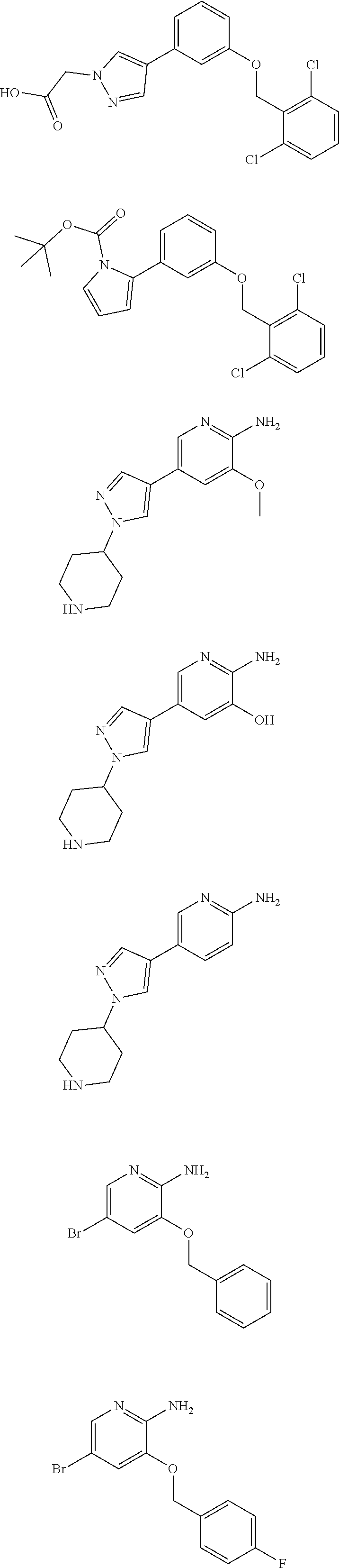

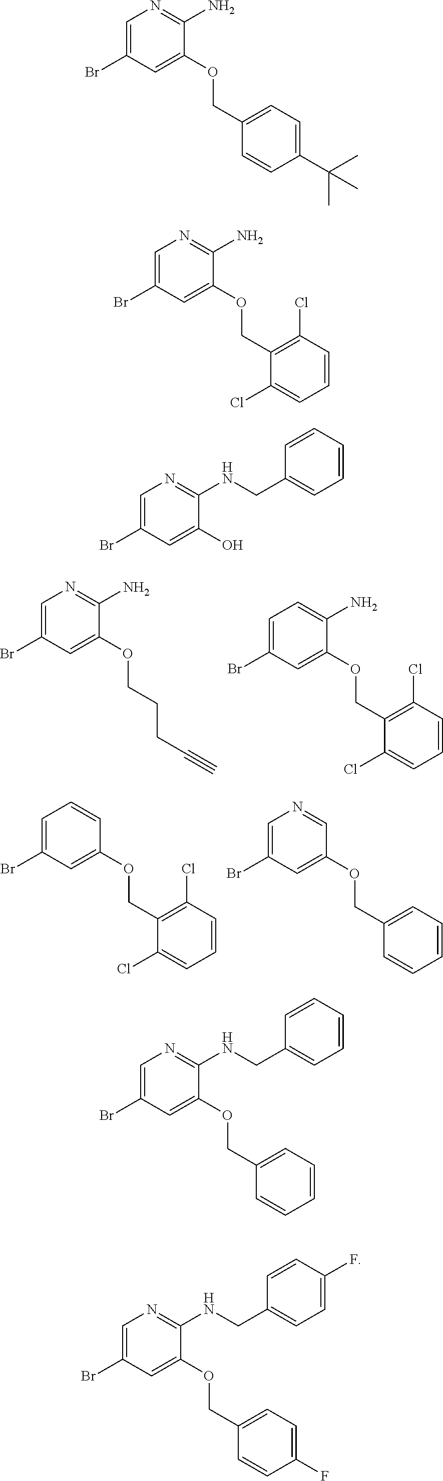

In one example embodiment, the compound is selected from the group consisting of:

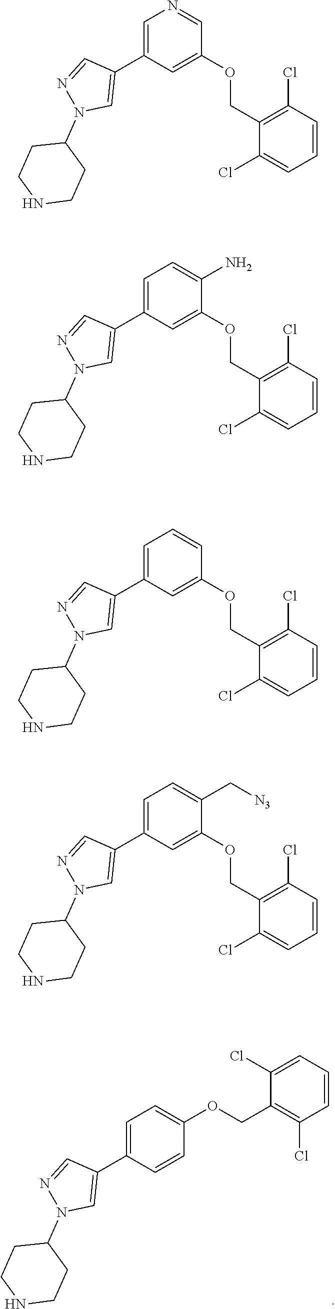 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

In one example embodiment, the compound is selected from the group consisting of:

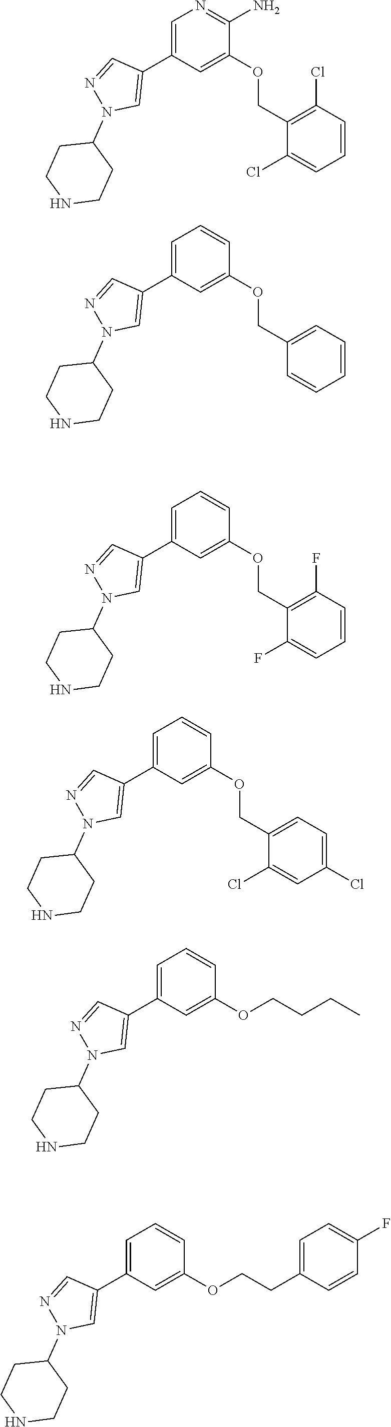 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

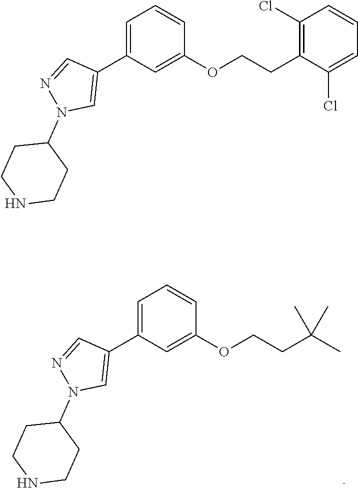 [SMILES: Clc1cccc(Cl)c1CCOc1cccc(-c2cnn(C3CCNCC3)c2)c1 ; CC(C)(C)CCOc1cccc(-c2cnn(C3CCNCC3)c2)c1]

In one example embodiment, compound is selected from the group consisting of:

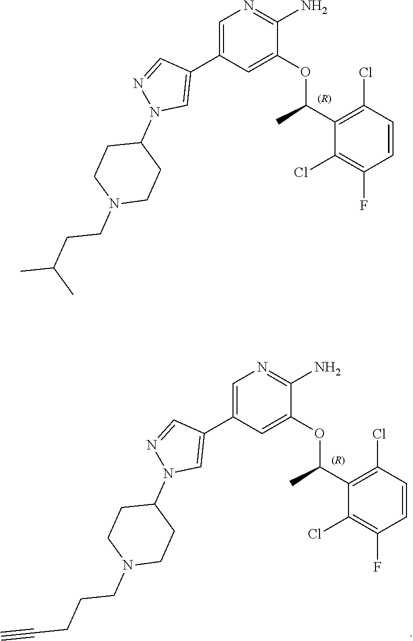

In one example embodiment, the compound is selected from the group consisting of:

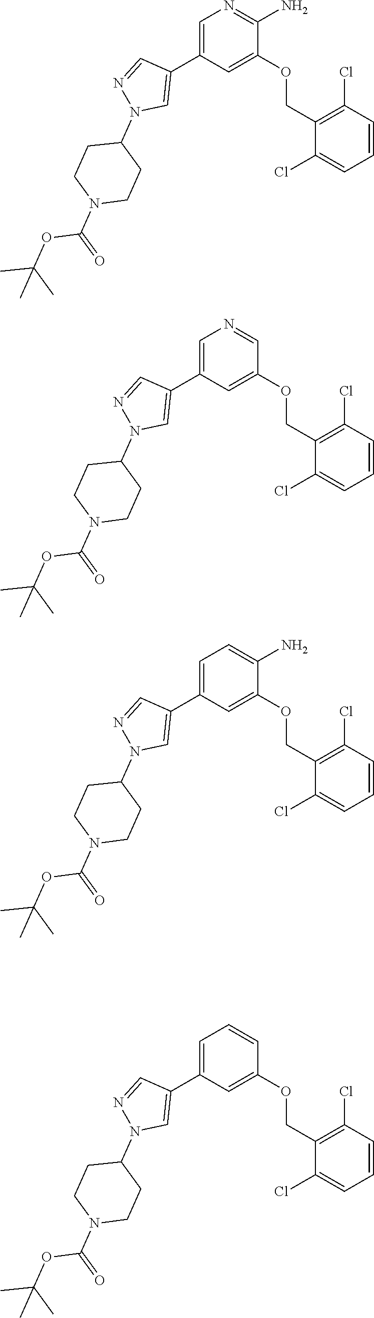 [SMILES: CC(C)(C)OC(=O)N1CCC(n2cc(-c3cnc(N)c(OCc4c(Cl)cccc4Cl)c3)cn2)CC1 ; CC(C)(C)OC(=O)N1CCC(n2cc(-c3cncc(OCc4c(Cl)cccc4Cl)c3)cn2)CC1 ; CC(C)(C)OC(=O)N1CCC(n2cc(-c3ccc(N)c(OCc4c(Cl)cccc4Cl)c3)cn2)CC1 ; CC(C)(C)OC(=O)N1CCC(n2cc(-c3cccc(OCc4c(Cl)cccc4Cl)c3)cn2)CC1]

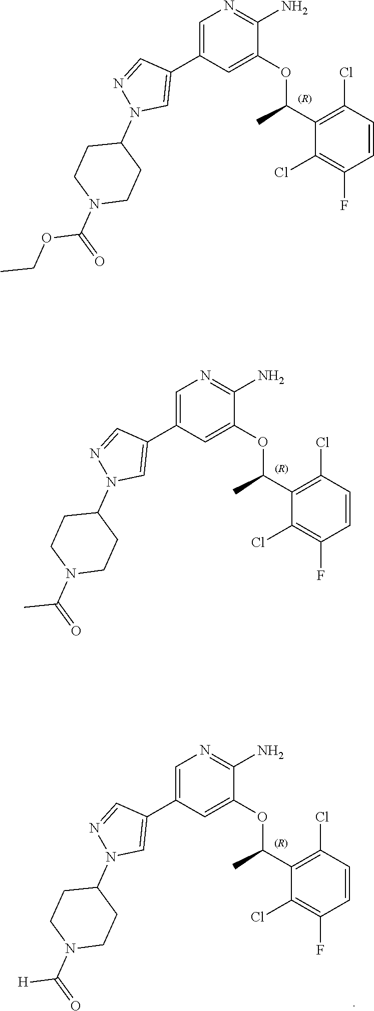

In one example embodiment, the compound is selected from the group consisting of:

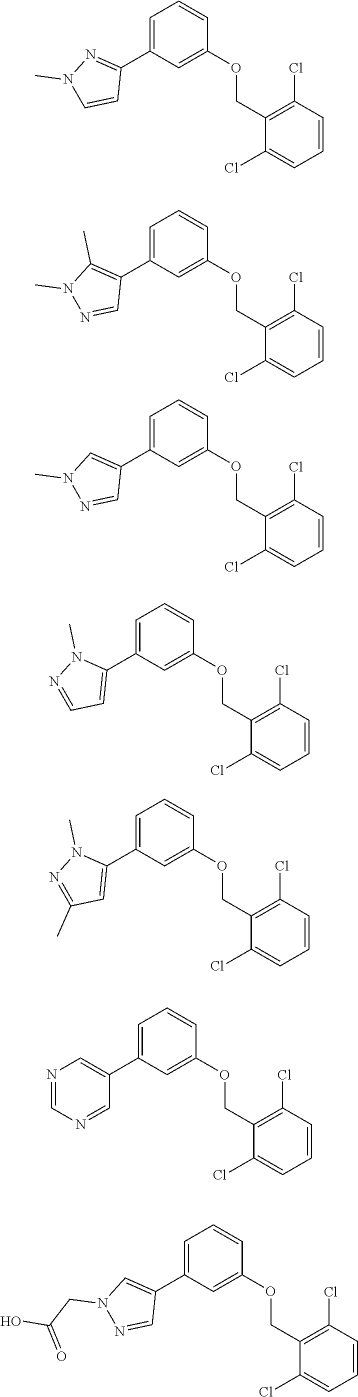

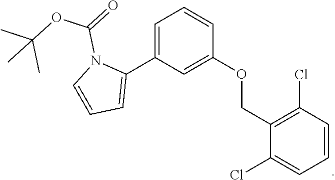 [SMILES: CC(C)(C)OC(=O)n1cccc1-c1cccc(OCc2c(Cl)cccc2Cl)c1]

In one example embodiment, the compound is selected from the group consisting of:

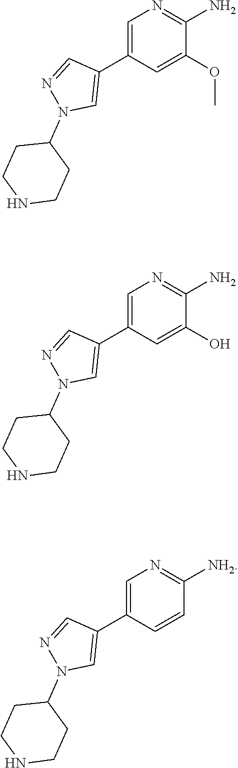 [SMILES: COc1cc(-c2cnn(C3CCNCC3)c2)cnc1N ; Nc1ncc(-c2cnn(C3CCNCC3)c2)cc1O ; Nc1ccc(-c2cnn(C3CCNCC3)c2)cn1]

In one example embodiment, the compound is selected from the group consisting of:

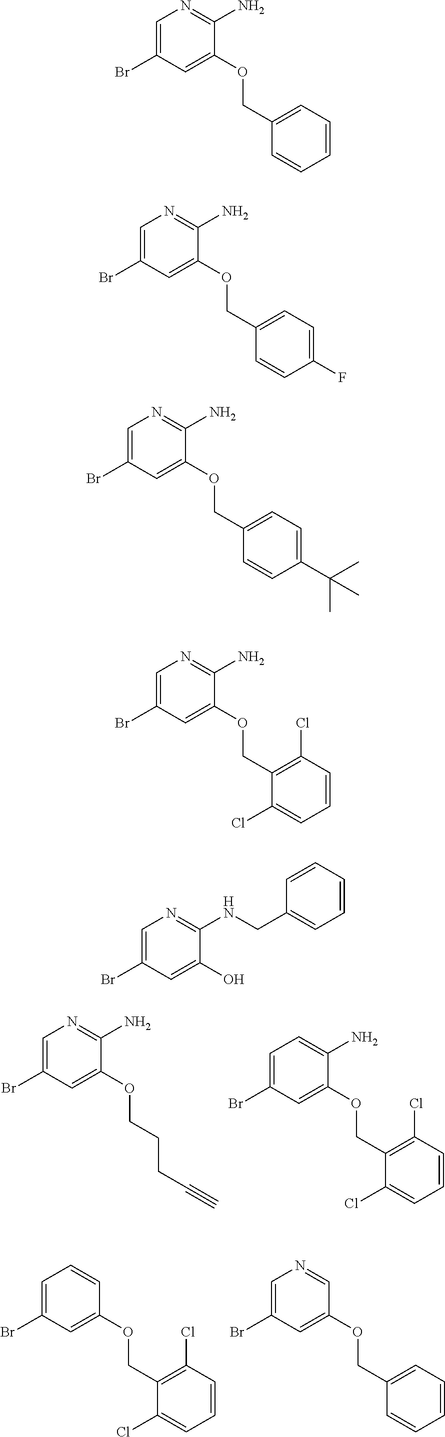

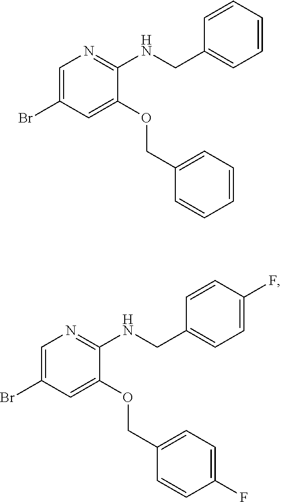 [SMILES: Brc1cnc(NCc2ccccc2)c(OCc2ccccc2)c1 ; Fc1ccc(CNc2ncc(Br)cc2OCc2ccc(F)cc2)cc1]

### Microbial Infections

The present disclosure relates to and/or involves microbial infections. In various aspects, compounds and pharmaceutical formulations of the present disclosure find value in methods for treating or preventing a microbial infection or for inducing anti-microbial activity such as, for example, in host cells, against microbes. In various example embodiments, microbial infections are selected from bacterial infections, viral infections, fungal infections, protozoa infections, and helminth infections.

### Bacterial Infections and Bacteria

In various aspects, the present disclosure relates to and/or involves compounds, pharmaceutical formulations, and methods of use of said compounds or said pharmaceutical formulations to treat or prevent a bacterial infection or to induce anti-microbial activity such as, for example, in host cells, against bacteria.

In one example embodiment, a bacterial infection caused by one or more bacteria, such as, for example, a set of bacteria. Various combinations of bacteria may form a set of bacteria. In one example embodiment, a set of bacteria includes one or more bacterial species, one or more strains of the same bacterial species, one or more phenotypes of the same bacterial species, one or more phenotypes of the same bacterial strain, or a combination thereof. In one example embodiment, a set of bacteria is a biological sample obtained from a subject in need thereof.

In one example embodiment, the bacterial infection is an acute bacterial infection, a chronic bacterial infection, a latent bacterial infection, a slow bacterial infection, a persistent bacterial infection, or any combination thereof.

In one example embodiment, the bacterial infection is caused by an antibiotic resistant bacteria, a dormant bacteria, a bacteria present in a biofilm, or any combination thereof. In one example embodiment, the bacterial infection is caused by a Gram-negative bacteria or a Gram-positive bacteria.

### Bacterial Biofilms

Bacterial biofilms exist in natural, medical, and engineering environments. Biofilms are recognized to be involved in many clinical infections and potentially contribute to the pathogenesis, especially in chronic infections. The biofilm may offer a selective advantage to a microorganism to ensure its survival or allow it a certain amount of time to exist in a dormant state until suitable growth conditions arise. This selective advantage could pose serious threats to human health. For example, biofilms are involved in 65% of human bacterial infections. Biofilms are also involved in prostatitis, biliary tract infections, urinary tract infections, cystitis, pyelonephritis, lung infections, sinus infections, ear infections, acne, and chronic wounds.

Biofilms contribute to a variety of medical conditions. Each year in the United States alone, over 7 million patients receive medical device implants, including central venous catheters, endotracheal tubes, mechanical heart valves, pacemakers, and prosthetic joints. Approximately one-half of these patients develop nosocomial infections, and approximately 80,000 deaths per year are attributed to nosocomial infections. Biofilms provide a structural matrix that facilitates bacterial adhesion to the inert surfaces of medical device implants and venous catheters. Microscopic studies confirm that central venous catheters are coated by bacteria embedded in biofilms. Unfortunately, more than 1 million patients develop urinary tract infections from such catheters.

Bacterial biofilms are clusters of bacteria that are attached to a surface and/or to each other and embedded in a self-produced matrix. The biofilm matrix consists of substances like proteins (e.g., fibrin), polysaccharide (e.g., alginate), as well as eDNA. In addition to the protection offered by the matrix, bacteria in biofilms can employ several survival strategies to evade the host defense systems. By staying dormant and hidden from the immune system, they may cause local tissue damage and later cause an acute infection. Within the biofilm, the bacteria adapt to environmental anoxia and nutrient limitation by exhibiting an altered metabolism, gene expression, and protein production, which can lead to a lower metabolic rate and a reduced rate of cell division. In addition, these adaptations make the bacteria more resistant to antimicrobial therapy by inactivating the antimicrobial targets or reducing the requirements for the cellular function that the antimicrobials interfere with. During a biofilm infection, simultaneous activation of both innate and acquired host immune responses may occur; neither of which are able to eliminate the biofilm pathogen, but instead accelerate collateral tissue damage. Consequently, biofilm-related diseases are typically persistent infections that develop slowly, are rarely resolved by the immune system, and respond inconsistently to antimicrobial treatments.

### Bacteria

In various example embodiments, the bacteria includes one or more bacterial species selected from an Acinetobacter species, an Actinobacillus species, an Actinomycetes species, an Actinomyces species, Aerococcus species an Aeromonas species, an Aggregatibacter species, an Anaplasma species, an Alcaligenes species, a Bacillus species, a Bacteroides species, a Bartonella species, a Bifidobacterium species, a Bordetella species, a Borrelia species, a Brucella species, a Burkholderia species, a Campylobacter species, a Capnocytophaga species, a Cardobacterium species, a Chlamydia species, a Citrobacter species, a Clostridium species, a Coxiella species, a Corynbacterium species, an Eikenella species, an Enterobacter species, an Escherichia species, an Enterococcus species, an Ehlichia species, an Epidermophyton species, an Erysipelothrix species, a Eubacterium species, a Francisella species, a Fusobacterium species, a Gardnerella species, a Gemella species, a Haemophilus species, a Helicobacter species, a Kingella species, a Klebsiella species, a Lactobacillus species, a Lactococcus species, a Listeria species, a Leptospira species, a Legionella species, a Leptospira species, Leuconostoc species, a Mannheimia species, a Microsporum species, a Micrococcus species, a Moraxella species, a Morganell species, a Mobiluncus species, a Micrococcus species, Mycobacterium species, a Mycoplasma species, a Nocardia species, a Neisseria species, a Pasteurelaa species, a Pediococcus species, a Peptostreptococcus species, a Pityrosporum species, a Plesiomonas species, a Prevotellaspecies, a Porphyromonas species, a Proteus species, a Providencia species, a Pseudomonas species, a Propionibacteriums species, a Rhodococcus species, a Rickettsia species, a Rhodococcus species, a Serratia species, a Stenotrophomonas species, a Salmonella species, a Serratia species, a Shigella species, a Staphylococcus species, a Streptococcus species, a Spirillum species, a Streptobacillus species, a Treponema species, a Tropheryma species, a Trichophyton species, an Ureaplasma species, a Veillonella species, a Vibrio species, a Yersinia species, and a Xanthomonas species.

In various example embodiments, the bacteria includes one or more bacterial species selected from Bacillus cereus, Burkholderia cenocepacia, Campylobacter jejuni, Escherichia coli (including uropathogenic Escherichia coli (UPEC)), Haemophilus influenzae, Helicobacter pylori, Klebsiella pneumoniae, Legionella pneumophila, Moraxella catarrhalis,Mycobacterium abscessus, Mycobacterium avium, Mycobacterium tuberculosis, Mycoplasma pneumoniae, Proteus mirabilis, Pseudomonas aeruginosa, Salmonella Enteritidis, Salmonella Typhi, Salmonella Typhimurium, Serratia marcescens, Staphylococcus aureus, Staphylococcus epidermis, Staphylococcus saprophytis, Streptococcus pneumonia, Streptococcus gallolyticus,and Vibrio cholerae.

In one example embodiment, the bacterial infection is a chronic or persistent bacterial infection. In one example embodiment, the bacterial infection is an infection of a single organ infection, a group of organs, or the entire body. In one example embodiment, the bacterial infection is a central nervous system infection, an eye infection, an ear infection, an upper respiratory tract infection, a lower respiratory tract infection, gastrointestinal infection, a heart infection, a gallbladder infection, a urinary tract infection, a skin infection, a blood infection, a bone infection, a hospital-acquired infection, a wound infection, a vaginal infection, a sexually transmitted disease, or any combination thereof.

In one example embodiment, the bacterial infection is a systemic infection. In one example embodiment, the bacterial infection is a systemic S. Typhi infection.

In one example embodiment, the bacterial infection is a lower respiratory infection. In one example embodiment, the lower respiratory infection is a lung infection. In one example embodiment, the lower respiratory infection is a chronic or persistent lung infection. In one example embodiment, the lower respiratory infection is caused by M. tuberculosis., S. aureus, P. aeruginosa, K. pneumoniae, L. pneumophila, S. pneumoniae, B. cenocepacia, H. influenzae, M. catarrhalis, M. pneumoniae, M. abscessus, M. avium, or any combination thereof.

In one example embodiment, the bacterial infection is a heart infection. In one example embodiment, the heart infection is endocarditis. In one example embodiment, the heart infection is chronic or persistent endocarditis. In one example embodiment, the heart infection is caused by S. aureus, other Staphylococcus spp, S. gallolyticus, other Streptococcus spp, Enterococcus spp, Haemophilus spp, Aggregatibacter spp, Cardiobacterium spp, Eikenella spp, Kingella spp, or any combination thereof.

In one example embodiment, the bacterial infection is a gallbladder infection. In one example embodiment, the gallbladder infection is an asymptomatic chronic or persistent gallbladder infection. In one example embodiment, the gallbladder infection is caused by S. Typhi.

In one example embodiment, the bacterial infection is a gastrointestinal infection. In one example embodiment, the gastrointestinal infection is caused by H. pylori, S. Enteritidis, S. Typhimurium, S. Typhi, S. aureus, Shigella sp., E. coli, V. cholerae, C. jejuni, Clostridium sp., B. cereus, Yersinia sp., or any combination thereof.

In one example embodiment, the bacterial infection is a urinary tract infection. In one example embodiment, the urinary tract infection is a chronic urinary tract infection. In one example embodiment, the urinary tract infection is caused by uropathogenic Escherichia coli(UPEC), K. pneumoniae, P. aeruginosa, S. saprophytis, S. aureus, P. mirabilis, S. marcescens, Enterobacter spp, or any combination thereof.

In one example embodiment, the bacterial infection is a bone infection. In one example embodiment, the bone infection is caused by S. aureus, other Staphylococcus spp, H. influenzae, Streptococcus spp, Pseudomonas spp, Enterobacter spp, or any combination thereof.

In one example embodiment, the bacterial infection is a wound infection. In one example embodiment, the bacterial infection is a chronic or persistent wound infection. In one example embodiment, the wound infection is caused by S. aureus, S. pneumoniae, E. coli, P. aeruginosa, Proteus mirablis, S. epidermis, Cornynebacterium spp, Klebsiella spp, other Staphylococcus spp, other Streptococcus spp, Enterococcus spp, or any combination thereof.

### Viral Infection and Viruses

In various aspects, the present disclosure relates to and/or involves compounds, pharmaceutical formulations, and methods of use of said compounds or said pharmaceutical formulations to treat or prevent a viral infection or to induce anti-microbial activity such as, for example, in host cells, against viruses.

In one example embodiment, the microbial infection is a viral infection caused by one or more viruses, such as, for example, a set of viruses. Various combinations of viruses may form a set of viruses. In one example embodiment, a set of viruses includes one or more viral species, one or more variants of the same viral species, one or more phenotypes of the same viral species, one or more phenotypes of the same viral variant, or a combination thereof. In one example embodiment, a set of viruses is a biological sample obtained from a subject in need thereof.

As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain example embodiments, a variant has developed a “specific group of mutations” that causes the variant to behave differently than that of the strain it originated from.

In one example embodiment, the viral infection is an acute viral infection, a chronic viral infection, a latent viral infection, a slow viral infection, a persistent viral infection, or any combination thereof. In one example embodiment, the viral infection is caused by an antimicrobial resistant virus and/or a dormant virus.

### Viruses

In various example embodiments, the virus includes one or more viral species selected from the Coronaviridae family. In various examples, the Coronaviridae family virus includes one or more viral species selected from the α-coronavirus genus, the β-coronavirus genus, the γ-coronavirus genus, and the δ-coronavirus genus. In various examples, the β-coronavirus genus virus includes one or more viral species selected from SARS-COV, SARS-CoV-2, MERS-COV, OC43, and HKU1.

### SARS-COV-2

In various example embodiments, the virus includes SARS-COV-2. In various aspects, the present disclosure relates to and/or involves compounds, pharmaceutical formulations, and methods of use of said compounds or said pharmaceutical formulations to treat or prevent a SARS-COV-2 infection or to induce anti-microbial activity such as, for example, in host cells, against SARS-COV-2.

As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from. While there are many thousands of variants of SARS-COV-2, (Koyama, Takahiko Koyama; Platt, Daniela; Parida, Laxmi (June 2020). “Variant analysis of SARS-COV-2 genomes”. Bulletin of the World Health Organization. 98: 495-504) there are also much larger groupings called clades. Several different clade nomenclatures for SARS-CoV-2 have been proposed. As of December 2020, GISAID, referring to SARS-COV-2 as hCoV-19 identified seven clades (O, S, L, V, G, GH, and GR) (Alm E, Broberg E K, Connor T, et al. Geographical and temporal distribution of SARS-COV-2 clades in the WHO European Region, January to June 2020 [published correction appears in Euro Surveill. 2020 August;25(33):]. Euro Surveill. 2020;25(32):2001410). Also as of December 2020, Nextstrain identified five (19A, 19B, 20A, 20B, and 20C) (Cited in Alm et al. 2020). Guan et al. identified five global clades (G614, S84, V251, I378 and D392) (Guan Q, Sadykov M, Mfarrej S, et al. A genetic barcode of SARS-COV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A .; Holmes, E. C.; O'Toole, Á.; et al. “A dynamic nomenclature proposal for SARS-COV-2 lineages to assist genomic epidemiology”. 5: 1403-1407).

Genetic variants of SARS-COV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present disclosure include variants of SARS-COV-2, particularly those having substitutions of therapeutic concern. Table 1 shows exemplary, non-limiting genetic substitutions in SARS-COV-2 variants.

TABLE 1

| | Common Pango Lineages with Spike Protein |
|---|---|
| Spike Protein Substitution | Substitutions |
| L452R | A.2.5, B.1, B.1.429, B.1.427, B.1.617.1, |
| | B.1.526.1, B.1.617.2, C.36.3 |
| E484K | B.1.1.318, B.1.1.7, B.1.351, B.1.525, |
| | B.1.526, B.1.621, B.1.623, P.1, P.1.1, P.1.2, |
| | R.1 |
| K417N, E484K, N501Y | B.1.351, B.1.351.3 |
| K417T, E484K, N501Y | P.1, P.1.1, P.1.2 |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-COV-2 lineages, known as the PANGO nomenclature. It is available at cov-lineages.org.

In some embodiments, the SARS-COV-2 variant is and/or includes: B.1.1.7, also known as Alpha (WHO) or UK variant, having the following spike protein substitutions: 69del, 70del, 144del, (E484K*), (S494P*), N501Y, A570D, D614G, P681H, T716I, S982A, and D1118H (K1191N*); B.1.351, also known as Beta (WHO) or South Africa variant, having the following spike protein substitutions: D80A, D215G, 241del, 242del, 243del, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13I, W152C, L452R, and D614G; B.1.617.2, also known as Delta (WHO) or India variant, having the following spike protein substitutions: T19R, (G142D), 156del, 157del, R158G, L452R, T478K, D614G, P681R, and D95ON; and P.1, also known as Gamma (WHO) or Japan/Brazil variant, having the following spike protein substitutions: L18F, T20N, P26S, D138Y, R190S, K417T, E484K, N501Y, D614G, H655Y, and T1027I, or any combination thereof.

In some embodiments, the SARS-COV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures.

In some embodiments, the SARS-COV-2 variant is classified and/or otherwise identified as a Variant of High Consequence (VHC) by the World Health Organization and/or the U.S. Centers for Disease Control. A variant of high consequence has clear evidence that prevention measures or medical countermeasures (MCMs) have significantly reduced effectiveness relative to previously circulating variants.

In some embodiments, the SARS-COV-2 variant is classified and/or otherwise identified as a Variant of Interest (VOI) by the World Health Organization and/or the U.S. Centers for Disease Control. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

In some embodiments, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In some embodiments the SARS-COV-2 variant is a VOC. In some embodiments, the SARS-COV-2 variant is or includes an Alpha variant (e.g., Pango lineage B.1.1.7), a Beta variant (e.g., Pango lineage B.1.351, B.1.351.1, B.1.351.2, and/or B.1.351.3), a Delta variant (e.g., Pango lineage B.1.617.2, AY.1, AY.2, AY.3 and/or AY.3.1); a Gamma variant (e.g., Pango lineage P.1, P.1.1, P.1.2, P.1.4, P.1.6, and/or P.1.7), or any combination thereof.

In some embodiments the SARS-Cov-2 variant is a VOI. In some embodiments, the SARS-COV-2 variant is or includes an Eta variant (e.g., Pango lineage B.1.525 (Spike protein substitutions A67V, 69del, 70del, 144del, E484K, D614G, Q677H, F888L)); an Iota variant (e.g., Pango lineage B.1.526 (Spike protein substitutions L5F, (D80G*), T95I, (Y144-*), (F157S*), D253G, (L452R*), (S477N*), E484K, D614G, A701V, (T859N*), (D950H*), (Q957R*))); a Kappa variant (e.g., Pango lineage B.1.617.1 (Spike protein substitutions (T95I), G142D, E154K, L452R, E484Q, D614G, P681R, Q1071H)); Pango lineage variant B.1.617.2 (Spike protein substitutions T19R, G142D, L452R, E484Q, D614G, P681R, D950N)), Lambda (e.g., Pango lineage C.37); Mu (e.g., Pango lineage B.1.621); or any combination thereof.

In some embodiments SARS-COV-2 variant is a VON. In some embodiments, the SARS-COV-2 variant is or includes Pango lineage variant P.1 (alias, B.1.1.28.1.) as described in Rambaut et al. 2020. Nat. Microbiol. 5:1403-1407) (spike protein substitutions: T20N, P26S, D138Y, R190S, K417T, E484K, N501Y, H655Y, TI027I)); an Alpha variant (e.g., Pango lineage B.1.1.7); a Beta variant (e.g., Pango lineage B.1.351, B.1.351.1, B.1.351.2, and/or B.1.351.3); Pango lineage variant B.1.617.2 (Spike protein substitutions T19R, G142D, L452R, E484Q, D614G, P681R, D950N)); an Eta variant (e.g., Pango lineage B.1.525); Pango lineage variant A.23.1 (as described in Bugembe et al. medRxiv. 2021. doi: https://doi.org/10.1101/2021.02.08.21251393) (spike protein substitutions: F157L, V367F, Q613H, P681R); or any combination thereof.

### Methods of Treating or Preventing a Microbial Infection

The present disclosure relates to and/or involves methods for treating or preventing a microbial infection. In one example embodiment, the method comprises: administering to a subject in need thereof a therapeutically effective amount of one or more inhibitors of one or more dehydrocholesterol reductases (e.g., DHCR7, DHCR14, and/or DHCR24) and/or lanosterol 14 alpha-demethylase (CYP51A1) of the post-lanosterol cholesterol biosynthesis pathway, pharmaceutically acceptable salts thereof, or any pharmaceutical combination thereof. In one example embodiment, the method comprises: administering to a subject in need thereof a therapeutically effective amount of a compound of the present disclosure, (R)-Crizotinib, (S)-Crizotinib, AY 9944, analogs thereof, pharmaceutically acceptable salts thereof, or any pharmaceutical combination thereof. In various example embodiments, the microbial infection is a bacterial infection or a viral infection. In one example embodiment, one or more inhibitors of one or more dehydrocholesterol reductases (e.g., DHCR7, DHCR14, and/or DHCR24) and/or lanosterol 14 alpha-demethylase (CYP51A1) of the post-lanosterol cholesterol biosynthesis pathway, pharmaceutically acceptable salts thereof, or any pharmaceutical combination thereof, are comprised in a pharmaceutical composition. In one example embodiment, one or more compounds of the present disclosure, (R)-Crizotinib, (S)-Crizotinib, AY 9944, analogs thereof, pharmaceutically acceptable salts thereof, or any pharmaceutical combination thereof, are comprised in a pharmaceutical composition.

Methods may utilize the inhibitors and compounds and compositions described herein, alone or in combination treatment, for intracellularly (e.g., within a cell or a population of cells) treating a microbial infection. In example embodiments, a microbial infection is caused by a microbe resistant to one or more antimicrobial drugs (e.g., multi-drug resistant bacteria such as, for example, K. pneumoniae). Advantageously, the compositions described herein may provide rapid broad spectrum rapid broad spectrum antimicrobial activity in host cells, and thus find use in methods of treating or preventing acute, chronic or persistent microbial infections.

In example embodiments, a microbial infection is a persistent microbial infection caused by a latent microbe (e.g., a non-growing microbe, a dormant microbe, or the like, such as, for example, uropathogenic Escheria coli, a microbe located within cells or tissues with low penetrance to one or more antimicrobial drugs (e.g., a persistent bacterial urinary tract infection), or a microbe otherwise persistent to one or more antimicrobial drugs. In example embodiments, a microbial infection is caused by a microbe for which resistance to one or more antimicrobial drugs is unknown (e.g., novel viruses such as, for example, SARS-COV-2).

In some embodiments, the subject in need thereof is at risk of being infected with a microbe, has been exposed to air, water, food, or a surface contaminated with a microbe, is suspected of having a microbial infection, has been exposed to an individual infected with a microbe, has been tested for a microbial infection, has tested positive for a microbial infection, and/or is symptomatic or asymptomatic of a microbial infection. In some embodiments, the subject in need thereof is immunocompromised (e.g., has an autoimmune disease), is susceptible to microbial infection (e.g., has diabetes), is symptomatic for or is an asymptomatic carrier for a chronic or a persistent microbial infection, or combinations thereof. Accordingly, in an example embodiment, the method of preventing a persistent microbial infection comprising administration of a compound as disclosed herein to a subject in need thereof, for example, a subject with an acute infection with potential to transition to persistent infection because of immune status, persistent infections, or prior history of antibiotic resistance.

### Methods of Treating or Preventing a Bacterial or Viral Infection

In one example embodiment, the method is a method for treating or preventing a bacterial or viral infection. In one example embodiment, the method comprises: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In one example embodiment, the one or more dehydrocholesterol reductases comprise DHCR7, DHCR14, and/or DHCR24. In one example embodiment, the method comprises: administering to a subject in need thereof a therapeutically effective amount of one or more compounds of the present disclosure, (R)-Crizotinib, (S)-Crizotinib, AY 9944, analogs thereof, pharmaceutically acceptable salts thereof, or any pharmaceutical combination thereof.

In one example embodiment, the method is effective for one or more of treating, preventing, delaying onset of, reducing the severity of, increasing the survival rate of, and reducing the recovery time from the bacterial or the viral infection.

In one example embodiment, the subject in need is or is not at risk of being infected with a bacteria or a virus, has or has not been exposed to air, water, food, or a surface contaminated with a bacteria or a virus, is or is not suspected of having a bacterial or a viral infection, has or has not been exposed to an individual infected with a bacteria or a virus, has or has not been tested for a bacterial or viral infection, has or has not tested positive for a bacterial or viral infection, and/or is symptomatic or is asymptomatic of a bacterial or viral infection.

### Methods of Inducing Anti-Microbial Activity Against a Microbial Infection

In an aspect, the present disclosure provides methods of inducing anti-microbial activity against a microbial infection. In an aspect, the methods comprise inducing an antimicrobial response in an infected host cell. The present disclosure indicates that inhibitors of one or more dehydrocholesterol reductases and/or lanosterol 14 alpha-demethylase (CYP51A1) of the post-lanosterol cholesterol biosynthesis pathway display widespread intracellular anti-microbial properties, thus, in one example embodiment, the method comprises: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In various example embodiments, the microbial infection is a bacterial infection or a viral infection.

The present disclosure indicates that (R)-crizotinib, (S)-crizotinib, AY 9944, and exemplary analogs thereof, display widespread intracellular anti-microbial properties, thus, in one example embodiment, the method comprises: administering to a subject in need thereof a therapeutically effective amount of one or more compounds of the present disclosure, (R)-Crizotinib, (S)-Crizotinib, AY 9944, analogs thereof, pharmaceutically acceptable salts thereof, or any pharmaceutical combination thereof. In various example embodiments, the microbial infection is a bacterial infection or a viral infection.

Accordingly, intracellular methods of treating a microbial infection in a subject in need thereof are provided, for example, which induce antimicrobial activity in host cells of the subject against various viral and bacterial infections, including multi-drug resistant infections, dormant infections, and unknown infections. The intracellular methods of treating, and the exemplary inhibitors and compounds disclosed herein, are effective at inducing host cells to kill multi-drug resistant microbes, latent microbes, otherwise inaccessible microbes, unknown microbes, or microbes for which antiviral drugs have not yet been identified.

### Methods of Inducing Anti-Microbial Activity Against a Bacterial or Viral Infection

In an aspect, the present disclosure provides a method for inducing anti-bacterial activity against a bacterial infection or anti-viral activity against a viral infection. In one example embodiment, the method comprises: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In one example embodiment, the antibacterial or antiviral activity is induced in host cells of the subject infected with a bacteria or a virus.

In one example embodiment, the method comprises: administering to a subject in need thereof a therapeutically effective amount of one or more compounds of the present disclosure, (R)-Crizotinib, (S)-Crizotinib, AY 9944, analogs thereof, pharmaceutically acceptable salts thereof, or any pharmaceutical combination thereof. In one example embodiment, the antibacterial or antiviral activity is induced in host cells of the subject infected with a bacteria or a virus.

### Pharmaceutical Formulations

In an aspect, the present disclosure provides pharmaceutical formulations for use in methods for treating or preventing a microbial infection or for inducing anti-microbial activity against a microbial infection. In an example embodiment, a pharmaceutical formulation comprises one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof. In an example embodiment, a pharmaceutical formulation comprises one or more compound(s) of the present disclosure, (R)-Crizotinib, (S)-Crizotinib, AY 9944, analogs thereof, pharmaceutically acceptable salts thereof, or pharmaceutical combinations thereof.

Described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

### Pharmaceutical Active Agents or Ingredients

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

### Pharmaceutically Acceptable Carriers and Secondary Ingredients and Agents

The pharmaceutical formulation can include a pharmaceutically acceptable carrier. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatoirenti-histamines, anti-infectives, anti-microbials, chemotherapeutics, and combinations thereof.

### Pharmaceutically Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are treating, preventing, delaying onset of, reducing the severity of, increasing the survival rate of, and reducing the recovery time from a microbial infection.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/ transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Routes of Delivery

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, MD, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, anti-microbials, chemotherapeutics, and combinations thereof. In some embodiment, the co-therapy or combination therapy additionally includes antibiotics, antivirals, antifungals, and combinations thereof.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

The invention is further described by the following numbered paragraphs

1. A compound or a salt thereof, wherein the compound has the structural formula:

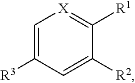

wherein X is C or N;
wherein R¹ is a hydrogen group, an amino group, an alkyl (including cycloalkyl) amino group, an alkyl azide (including a cycloalkyl azide) group, an alkylaryl (including cycloalkylaryl) amino group, an alkylaryloxy (including cycloalkylaryloxy) group, or an aryl amino group, and wherein R¹ is optionally independently substituted at one or more aryl ring positions with one or more halogen groups;
wherein R² is a hydrogen group, a hydroxyl group, an alkoxy (including cycloalkoxy) group, an alkynyl alkoxy (including alkynyl cycloalkoxy) group, or an aryl alkoxy (including aryl cycloalkoxy) group, and wherein, optionally, the aryl alkoxy (including aryl cycloalkoxy) group is independently substituted at one or more aryl ring positions with one or more halogen groups;
wherein R³ is a halogen group or a ring carbon of an aromatic heterocyclic amine group, wherein, optionally, the aromatic heterocyclic amine group is independently substituted at one or more heterocyclic ring positions with one or more alkyl (including cycloalkyl) groups, alkoxycarbonyl (including cycloalkoxycarbonyl) groups, carboxylic acid groups, and/or nonaromatic heterocyclic amine groups; and
wherein the compound excludes (R)-Crizotinib or (S)-Crizotinib.
   2. The compound of paragraph 1, wherein the aryl alkoxy group of R² is a hydrogen group, a C₄-C₆ alkyl ether group, a benzyloxy group, a 2-(phenyl)ethoxy group, or a chiral 1-(phenyl)ethoxy group, and wherein R² is optionally independently substituted at one or more aryl ring positions with one or more fluorine groups and/or one or more chlorine groups.
   3. The compound of paragraph 1 to 2, wherein the aryl alkoxy group of R² is a 1-(2,6-dichloro-3-fluorophenylethoxy) group comprising an (R) configuration, a 2-(2,6-dichloro-phenylethoxy) group, or a (2,6-dichloro-3-fluorobenzyloxy) group.
   4. The compound of any one of paragraphs 1 to 3, wherein the aromatic heterocyclic amine group of R³ is a pyrazole group, a pyrrole group, or a pyrimidine group.
   5. The compound of any one of paragraphs 1 to 4, wherein the nonaromatic heterocyclic amine group of R³ is substituted at one or more positions with one or more piperidine groups optionally independently substituted at one or more positions with one or more formyl groups, alkyl (including cycloalkyl) groups, alkylalkynyl (including cycloalkylalkynyl) groups, and/or alkoxycarbonyl (including cycloalkycarbonyl) groups.
   6. The compound of any one of paragraphs 1 to 5, wherein the aromatic heterocyclic amine group R³ is a 1-(piperidin-4-yl)pyrazol-4-yl group, a 1-(N-alkylpiperidin-4-yl)pyrazol-4-yl group, or a 1-(N-alkylalkynypiperidin-4-yl)pyrazol-4-yl group.
   7. The compound of any one of paragraphs 1 to 6, wherein the compound is selected from the group consisting of:

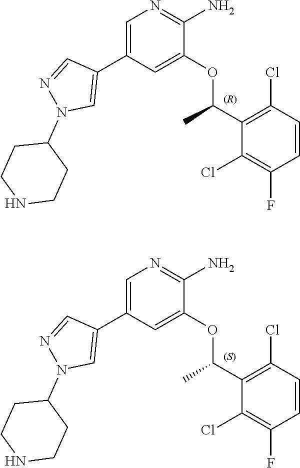

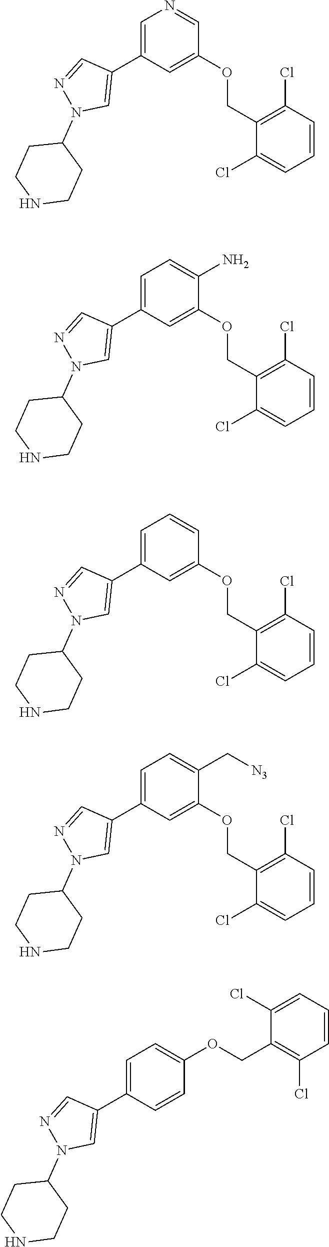 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

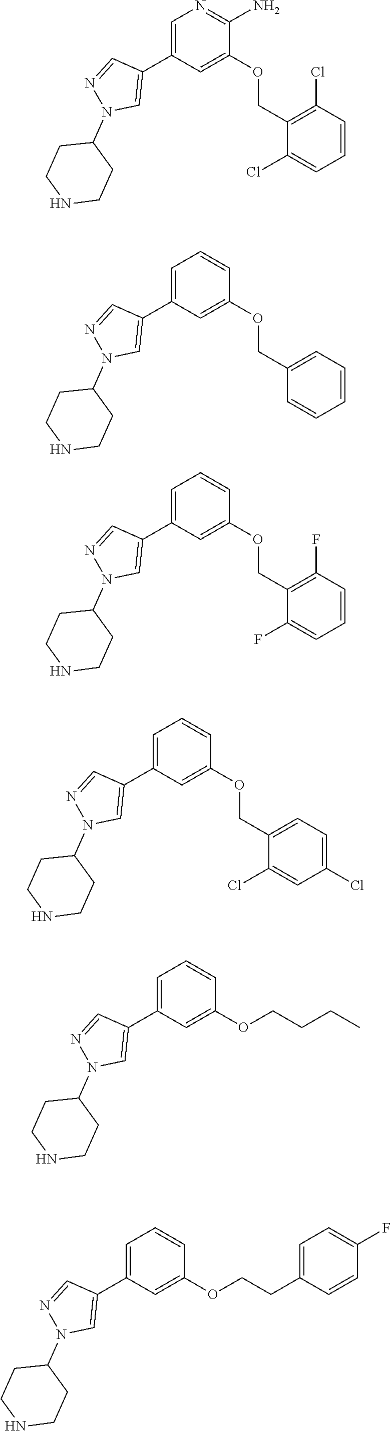 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

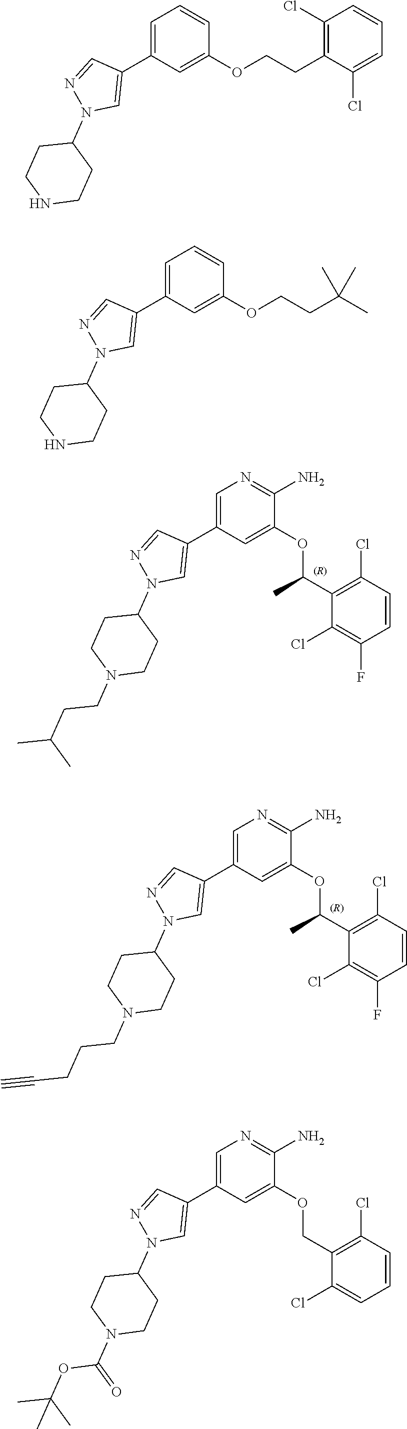

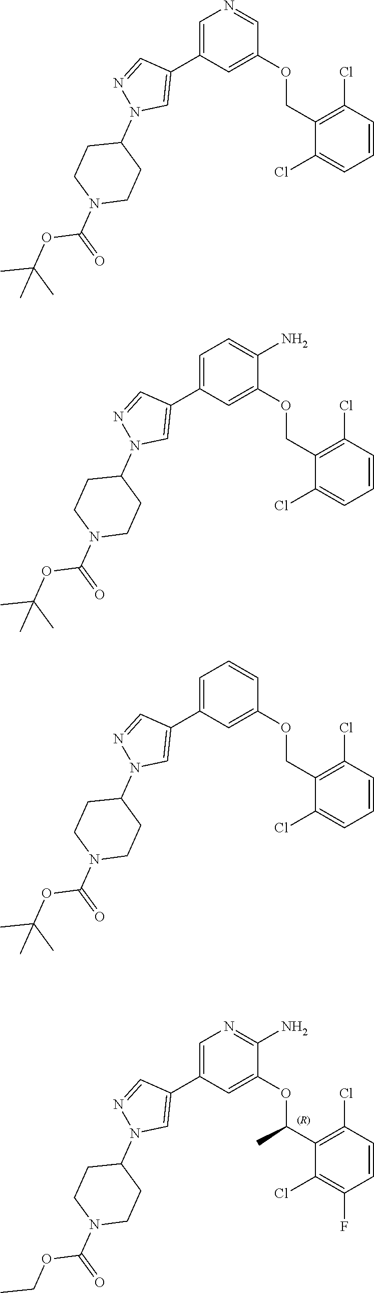

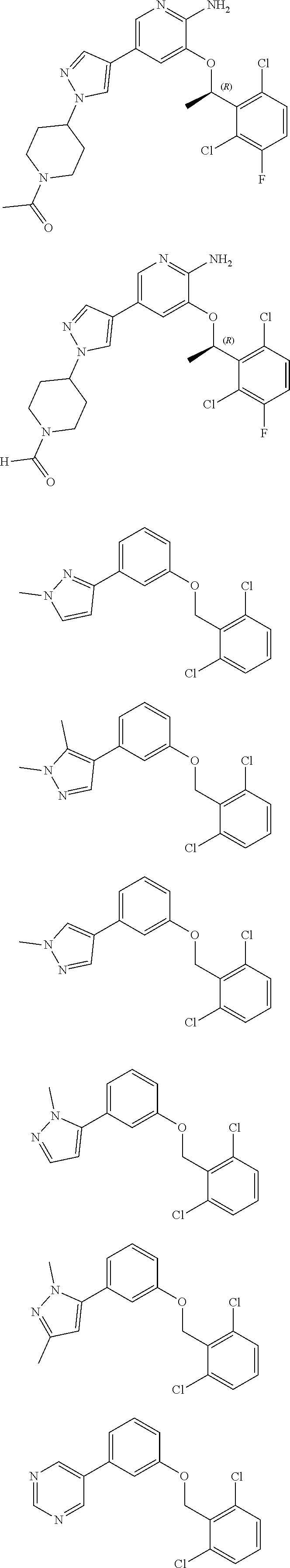

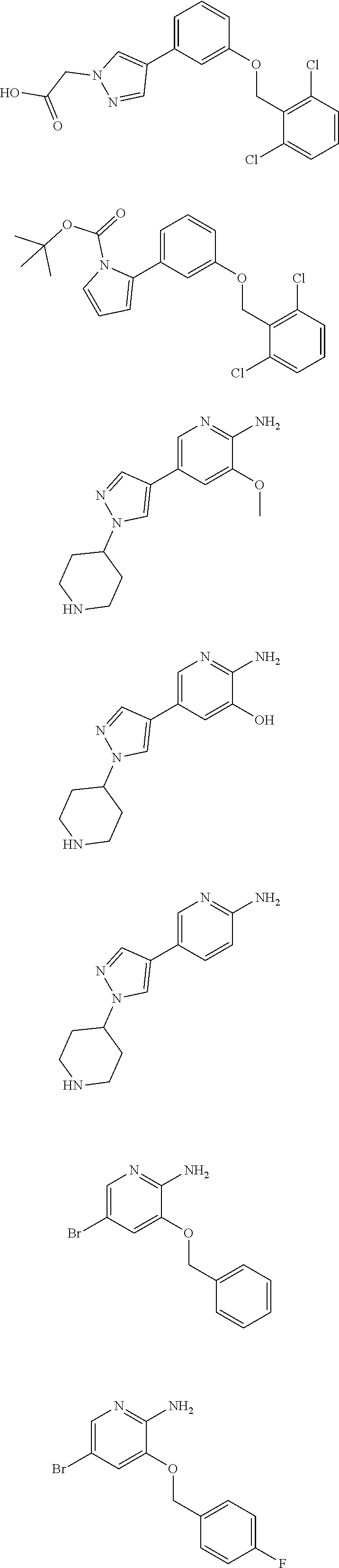

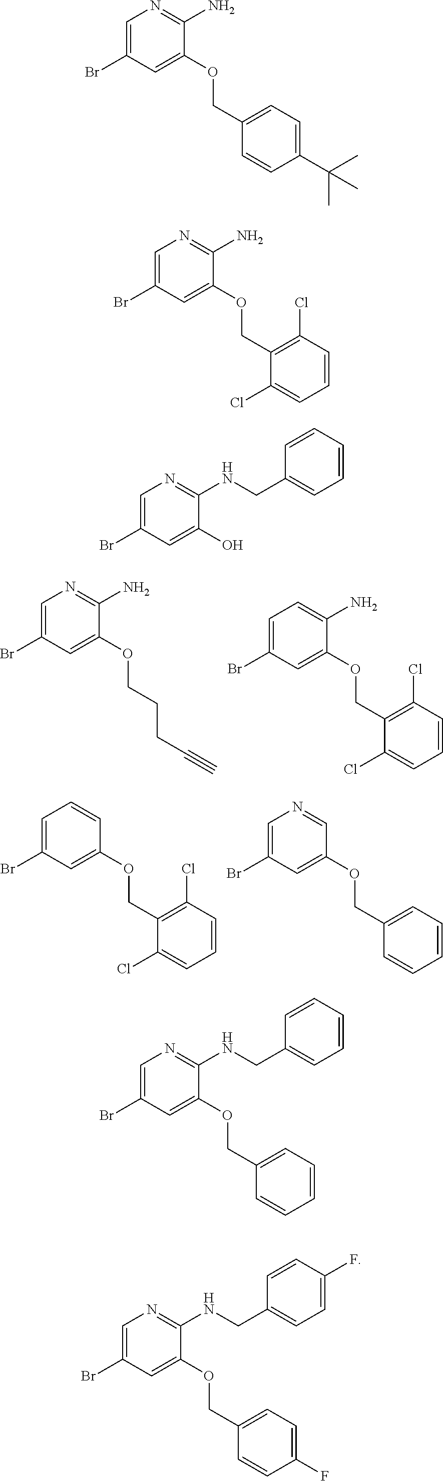

8. The compound of any one of paragraphs I to 7, wherein the compound is selected from the group consisting of:

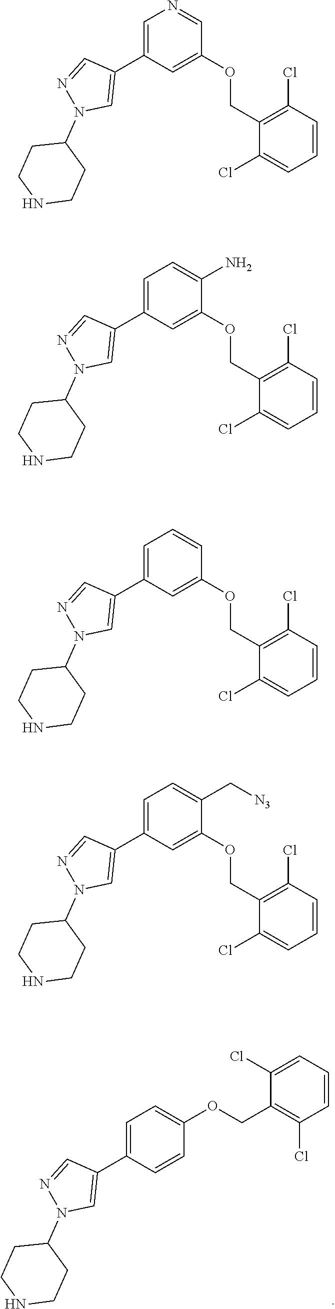 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

9. The compound of any one of paragraphs 1 to 7, wherein the compound is selected from the group consisting of:

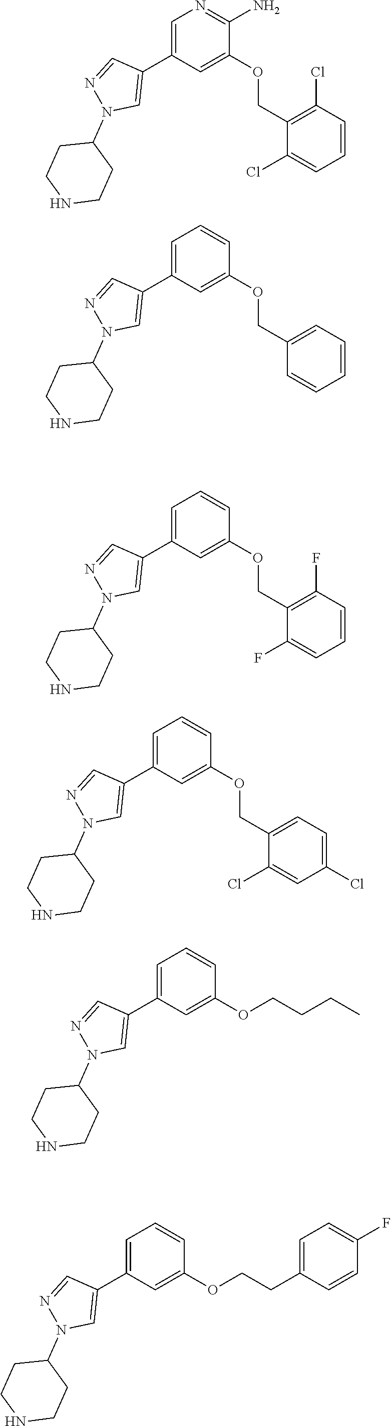 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

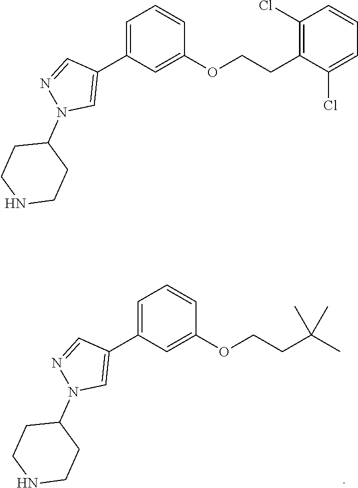 [SMILES: Clc1cccc(Cl)c1CCOc1cccc(-c2cnn(C3CCNCC3)c2)c1 ; CC(C)(C)CCOc1cccc(-c2cnn(C3CCNCC3)c2)c1]

10. The compound of any one of paragraphs 1 to 7, wherein the compound is selected from the group consisting of:

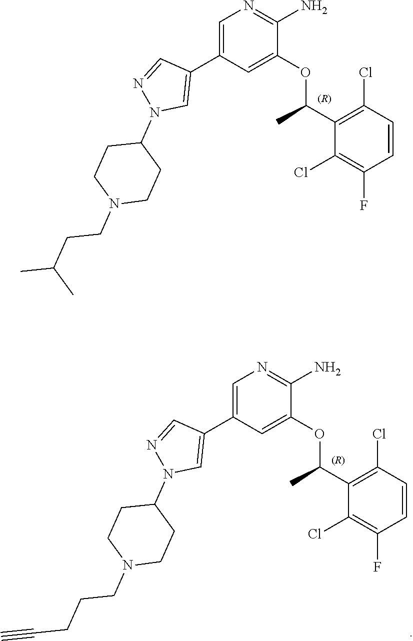

11. The compound of any one of paragraphs 1 to 7, wherein the compound is selected from the group consisting of:

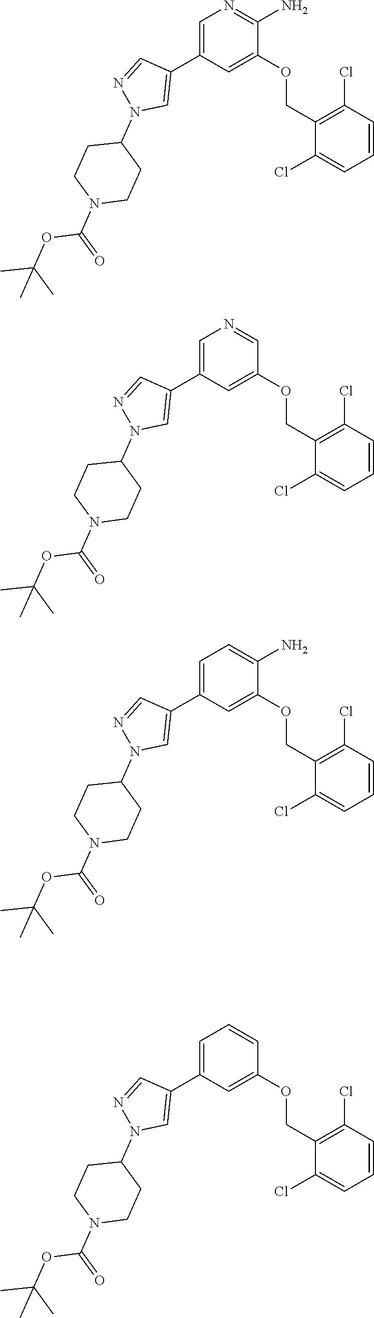 [SMILES: CC(C)(C)OC(=O)N1CCC(n2cc(-c3cnc(N)c(OCc4c(Cl)cccc4Cl)c3)cn2)CC1 ; CC(C)(C)OC(=O)N1CCC(n2cc(-c3cncc(OCc4c(Cl)cccc4Cl)c3)cn2)CC1 ; CC(C)(C)OC(=O)N1CCC(n2cc(-c3ccc(N)c(OCc4c(Cl)cccc4Cl)c3)cn2)CC1 ; CC(C)(C)OC(=O)N1CCC(n2cc(-c3cccc(OCc4c(Cl)cccc4Cl)c3)cn2)CC1]

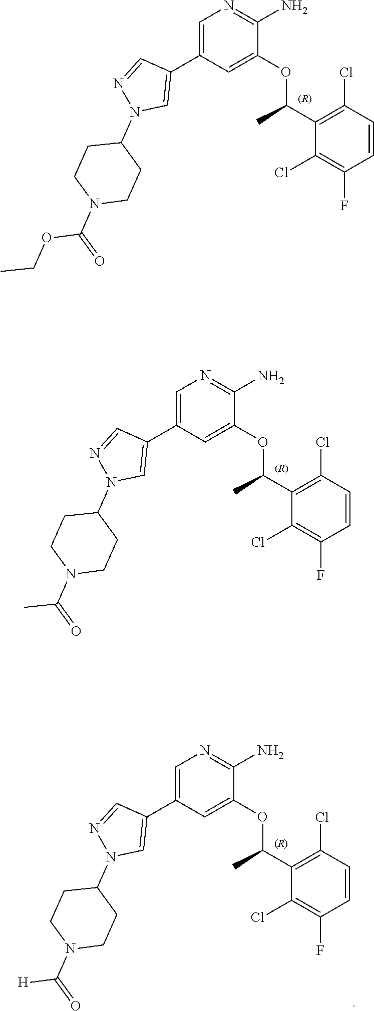

12. The compound of any one of paragraphs 1 to 7, wherein the compound is selected from the group consisting of:

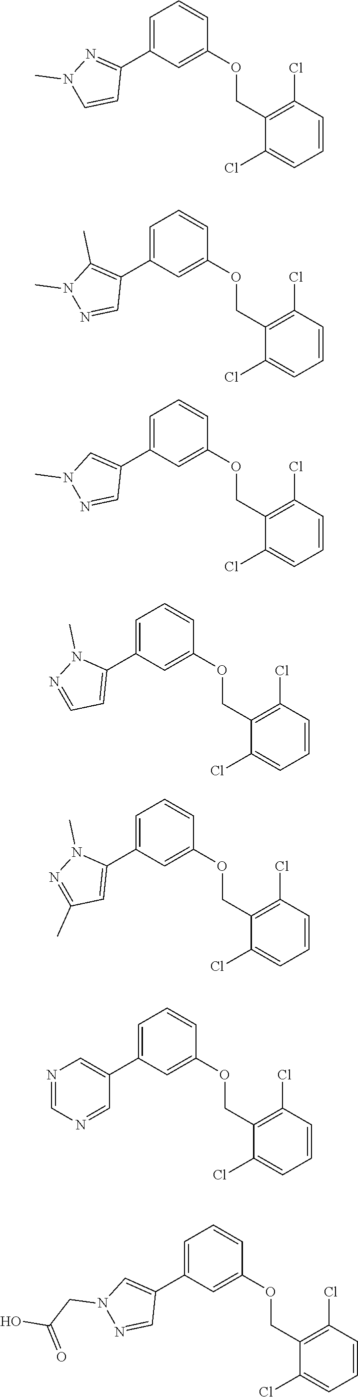

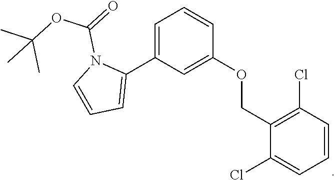 [SMILES: CC(C)(C)OC(=O)n1cccc1-c1cccc(OCc2c(Cl)cccc2Cl)c1]

13. The compound of any one of paragraphs 1 to 7, wherein the compound is selected from the group consisting of:

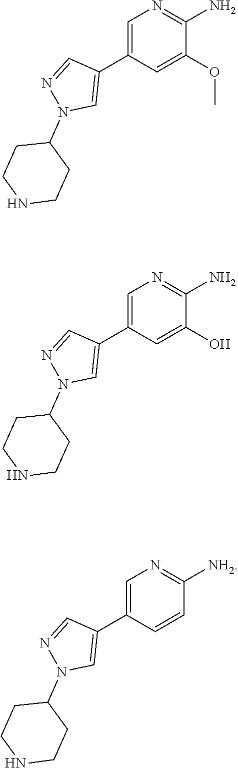 [SMILES: COc1cc(-c2cnn(C3CCNCC3)c2)cnc1N ; Nc1ncc(-c2cnn(C3CCNCC3)c2)cc1O ; Nc1ccc(-c2cnn(C3CCNCC3)c2)cn1]

14. The compound of any one of paragraphs 1 to 7, wherein the compound is selected from the group consisting of:

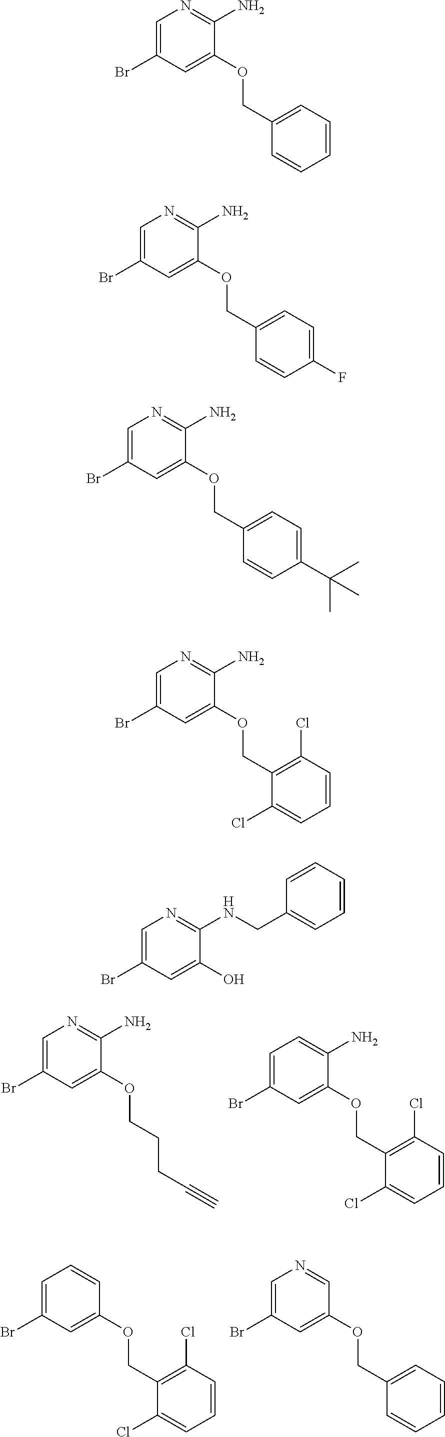

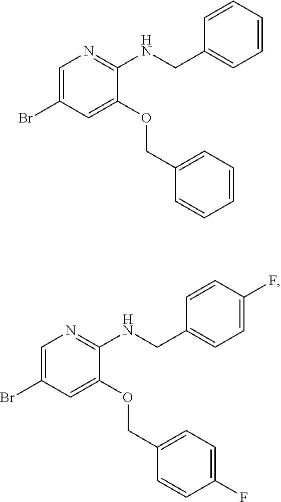 [SMILES: Brc1cnc(NCc2ccccc2)c(OCc2ccccc2)c1 ; Fc1ccc(CNc2ncc(Br)cc2OCc2ccc(F)cc2)cc1]

15. A pharmaceutical formulation comprising one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof.
16. The pharmaceutical formulation of claim 15, wherein the one or more dehydrocholesterol reductases comprise DHCR7, DHCR14, and/or DHCR24.
17. A pharmaceutical formulation comprising one or more compound(s) of any one of the preceding paragraphs, (R)-Crizotinib, (S)-Crizotinib, AY 9944, analogs thereof, pharmaceutically acceptable salts thereof, or any pharmaceutical combination thereof.
18. A method for treating or preventing a bacterial infection, comprising: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof.
19. The method of claim 18, wherein the one or more dehydrocholesterol reductases comprise DHCR7, DHCR 14, and/or DHCR24.
20. A method for treating or preventing a bacterial infection, comprising: administering to a subject in need thereof a therapeutically effective amount of one or more compounds selected from any one of paragraphs 1 to 14, (R)-Crizotinib, (S)-Crizotinib, AY 9944, analogs thereof, pharmaceutically acceptable salts thereof, or any pharmaceutical combination thereof.
21. The method of any one of paragraphs 18 to 20, wherein the bacterial infection is an acute bacterial infection, a chronic bacterial infection, a latent bacterial infection, a slow bacterial infection, a persistent bacterial infection, or any combination thereof.
22. The method of any one of paragraphs 18 to 21, wherein the bacterial infection is caused by an antibiotic resistant bacteria, a dormant bacteria, a bacteria present in a biofilm, or any combination thereof.
23. The method of any one of paragraphs 18 to 22, wherein the bacterial infection is caused by a Gram-negative bacteria or a Gram-positive bacteria.
24. The method of any one of paragraphs 18 to 23, wherein the bacterial infection is a central nervous system infection, an eye infection, an ear infection, an upper respiratory tract infection, a lower respiratory tract infection, gastrointestinal infection, a heart infection, a gallbladder infection, a urinary tract infection, a skin infection, a blood infection, a bone infection, a hospital-acquired infection, a wound infection, a vaginal infection, a sexually transmitted disease, or any combination thereof.
25. The method of paragraph 24, wherein the lower respiratory infection is caused by M. tuberculosis, S. aureus, P. aeruginosa, K. pneumoniae, L. pneumophila, S. pneumoniae, B. cenocepacia, H. influenzae, M. catarrhalis, M. pneumoniae, M. abscessus, M. avium, or any combination thereof.
26. The method of paragraph 24, wherein the heart infection is caused by S. aureus, other Staphylococcus spp, S. gallolyticus, other Streptococcus spp, Enterococcus spp, Haemophilus spp, Aggregatibacter spp, Cardiobacterium spp, Eikenella spp, Kingella spp, or any combination thereof.
27. The method of paragraph 24, wherein the gallbladder infection is caused by S. Typhi.
28. The method of paragraph 24, wherein the gastrointestinal infection is caused by H. pylori, S. Enteritidis, S. Typhimurium, S. Typhi, S. aureus, Shigella sp., E. coli, V. cholerae, C. jejuni, Clostridium sp., B. cereus, Yersinia sp., or any combination thereof.
29. The method of paragraph 24, wherein the urinary tract infection is caused by uropathogenic Escherichia coli (UPEC), K. pneumoniae, P. aeruginosa, S. saprophytis, S. aureus, P. mirabilis, S. marcescens, Enterobacter spp, or any combination thereof.
30. The method of paragraph 24, wherein the bone infection is caused by S. aureus, other Staphylococcus spp, H. influenzae, Streptococcus spp, Pseudomonas spp, Enterobacter spp, or any combination thereof.
31. The method of paragraph 24, wherein the wound infection is caused by S. aureus, S. pneumonia, E. coli, P. aeruginosa, Proteus mirablis, S. epidermis, Cornynebacterium spp, Klebsiella spp, other Staphylococcus spp, other Streptococcus spp, Enterococcus spp, or any combination thereof.
32. The method of any one of paragraphs 18 to 31, wherein the bacterial infection is a persistent bacterial infection.
33. The method of paragraph 32, wherein the persistent bacterial infection is a persistent lung infection, a persistent heart infection, a persistent gall bladder infection, a persistent bone infection, a persistent wound infection, or a persistent urinary tract infection, or any combination thereof.
34. The method of paragraph 32 or 33, wherein the persistent bacterial infection is caused by a Gram-negative bacteria.
35. The method of any one of paragraphs 32 to 34, wherein the persistent bacterial infection is a persistent urinary tract infection.
36. The method of paragraph 35, wherein the persistent urinary tract infection is caused by UPEC, K. pneumoniae, or P. aeruginosa, or any combination thereof.
37. A method for inducing antimicrobial activity against a bacterial infection comprising: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof.
38. The method of claim 37, wherein the one or more dehydrocholesterol reductases comprise DHCR7, DHCR14, and/or DHCR24.
39. A method for inducing antimicrobial activity against a bacterial infection comprising: administering to a subject in need thereof a therapeutically effective amount of one or more compounds selected from any one of paragraphs 1 to 14, (R)-Crizotinib, (S)-Crizotinib, AY 9944, analogs thereof, pharmaceutically acceptable salts thereof, or any pharmaceutical combination thereof.
40. The method of any one of paragraphs 37 to 39, wherein the antimicrobial activity is induced in host cells of the subject infected with a bacteria.
41. The method of any one of paragraphs 37 to 40, wherein the bacterial infection is an acute bacterial infection, a chronic bacterial infection, a latent bacterial infection, a slow bacterial infection, a persistent bacterial infection, or any combination thereof.
42. The method of any one of paragraphs 37 to 41, wherein the bacterial infection is caused by an antimicrobial resistant bacteria, a dormant bacteria, a bacteria present in a biofilm, or any combination thereof.
43. The method of any one of paragraphs 37 to 42, wherein the bacterial infection is caused by a Gram-negative bacteria or a Gram-positive bacteria.
44. The method of any one of paragraphs 37 to 43, wherein the bacterial infection is a central nervous system infection, an eye infection, an ear infection, an upper respiratory tract infection, a lower respiratory tract infection, gastrointestinal infection, a heart infection, a gallbladder infection, a urinary tract infection, a skin infection, a blood infection, a bone infection, a hospital-acquired infection, a wound infection, a vaginal infection, a sexually transmitted disease, or any combination thereof.
45. The method of paragraph 44, wherein the lower respiratory infection is caused by M. tuberculosis., S. aureus, P. aeruginosa, K. pneumoniae, L. pneumophila, S. pneumoniae, B. cenocepacia, H. influenzae, M. catarrhalis, M. pneumoniae, M. abscessus, M. avium, or any combination thereof.
46. The method of paragraph 44, wherein the heart infection is caused by S. aureus, other Staphylococcus spp, S. gallolyticus, other Streptococcus spp, Enterococcus spp, Haemophilus spp, Aggregatibacter spp, Cardiobacterium spp, Eikenella spp, Kingella spp, or any combination thereof.
47. The method of paragraph 44, wherein the gallbladder infection is caused by S. Typhi.
48. The method of paragraph 44, wherein the gastrointestinal infection is caused by H.pylori, S. Enteritidis, S. Typhimurium, S. Typhi, S. aureus, Shigella sp., E. coli, V. cholerae, C. jejuni, Clostridium sp., B. cereus, Yersinia sp., or any combination thereof.
49. The method of paragraph 44, wherein the urinary tract infection is caused by uropathogenic Escherichia coli (UPEC), K. pneumoniae, P. aeruginosa, S. saprophytis, S. aureus, P. mirabilis, S. marcescens, Enterobacter spp, or any combination thereof.
50. The method of paragraph 44, wherein the bone infection is caused by S. aureus, other Staphylococcus spp, H. influenzae, Streptococcus spp, Pseudomonas spp, Enterobacter spp, or any combination thereof.
51. The method of paragraph 44, wherein the wound infection is caused by S. aureus, S. pneumonia, E. coli, P. aeruginosa, Proteus mirablis, S. epidermis, Cornynebacterium spp, Klebsiella spp, other Staphylococcus spp, other Streptococcus spp, Enterococcus spp, or any combination thereof.
52. The method of any one of paragraphs 37 to 51, wherein the bacterial infection is a persistent bacterial infection.
53. The method of paragraph 52, wherein the persistent bacterial infection is a persistent lung infection, a persistent heart infection, a persistent gall bladder infection, a persistent bone infection, a persistent wound infection, or a persistent urinary tract infection, or any combination thereof.
54. The method of paragraph 52 or 53, wherein the persistent bacterial infection is caused by a Gram-negative bacteria.
55. The method of any one of paragraphs 52 to 54, wherein the persistent bacterial infection is a persistent urinary tract infection.
56. The method of paragraph 55, wherein the persistent urinary tract infection is caused by UPEC, K. pneumoniae, or P. aeruginosa, or any combination thereof.
57. A method for treating or preventing a viral infection, comprising: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof.
58. The method of claim 57, wherein the one or more dehydrocholesterol reductases comprise DHCR7, DHCR14, and/or DHCR24.
59. A method of treating or preventing a viral infection comprising: administering to a subject in need thereof a therapeutically effective amount of one or more compounds selected from any one of paragraphs 1 to 14, (R)-Crizotinib, (S)-Crizotinib, AY 9944, analogs thereof, pharmaceutically acceptable salts thereof, or any pharmaceutical combination thereof.
60. The method of paragraph 59, wherein the viral infection is an acute viral infection, a chronic viral infection, a latent viral infection, a slow viral infection, a persistent viral infection, or any combination thereof.
61. The method of paragraph 59 or 60, wherein the viral infection is caused by an antimicrobial resistant virus and/or a dormant virus.
62. The method of any one of paragraph 59 to 61 wherein the viral infection is caused by a Coronaviridae virus.
63. The method of paragraph 62, wherein the Coronaviridae virus is a from β-coronavirus.
64. The method of paragraph 63, wherein the β-coronavirus is SARS-COV-2.
65. A method for inducing antimicrobial activity against a viral infection comprising: administering to a subject in need thereof a therapeutically effective amount of one or more inhibitors of one or more dehydrocholesterol reductases and/or lanosterol 14 alpha-demethylase (CYP51A1) of the post-lanosterol cholesterol biosynthesis pathway, pharmaceutically acceptable salts thereof, or any pharmaceutical combination thereof.
66. The method of claim 65, wherein the one or more dehydrocholesterol reductases comprise DHCR7, DHCR14, and/or DHCR24.
67. A method for inducing antimicrobial activity against a viral infection comprising: administering to a subject in need thereof a therapeutically effective amount of one or more compounds selected from any one of paragraphs 1 to 14, (R)-Crizotinib, (S)-Crizotinib, AY 9944, analogs thereof, pharmaceutically acceptable salts thereof, or any pharmaceutical combination thereof.
68. The method of paragraph 67, wherein the antimicrobial activity is induced in host cells of the subject infected with a virus.
69. The method of paragraph 67 or 68, wherein the viral infection is an acute viral infection, a chronic viral infection, a latent viral infection, a slow viral infection, a persistent viral infection, or any combination thereof.
70. The method of any one of paragraphs 67 to 69, wherein the viral infection is caused by an antimicrobial resistant virus and/or a dormant virus.
71. The method of any one of paragraphs 67 to 70, wherein the viral infection is cause by a Coronaviridae virus.
72. The method of paragraph 71, wherein the Coronaviridae virus is a from β-coronavirus.
73. The method of paragraph 72, wherein the β-coronavirus is SARS-COV-2.

Further embodiments are illustrated in the following Examples which are given for illustrative purposes only and are not intended to limit the scope of the invention.

### EXAMPLES

### Example 1—Intracellular Infection Induces Antibiotic Tolerance in K. pneumoniae

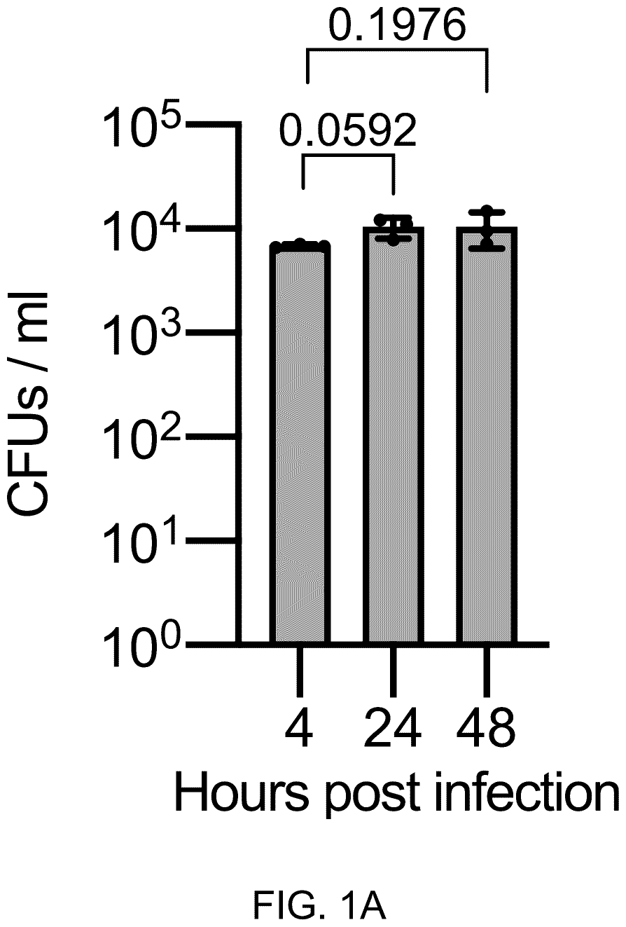

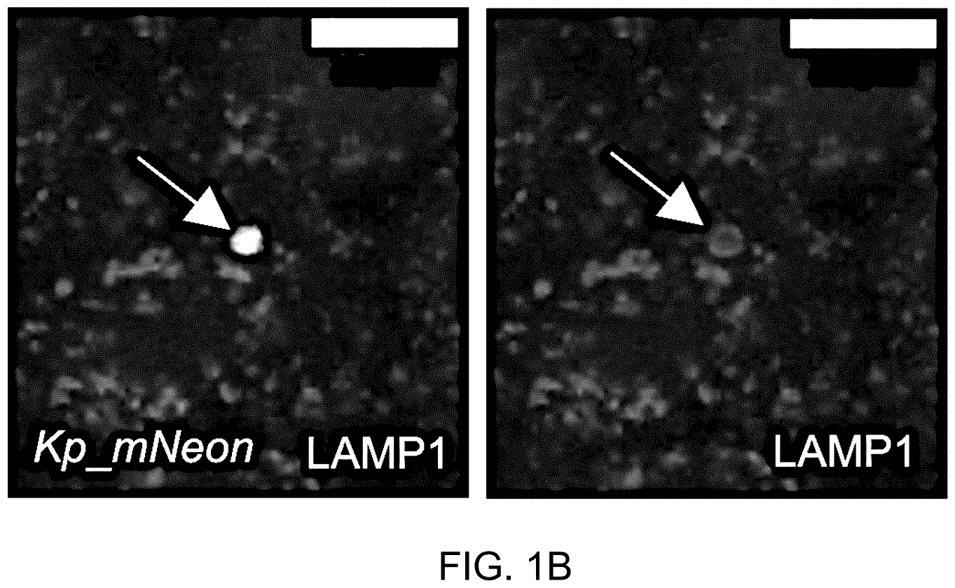

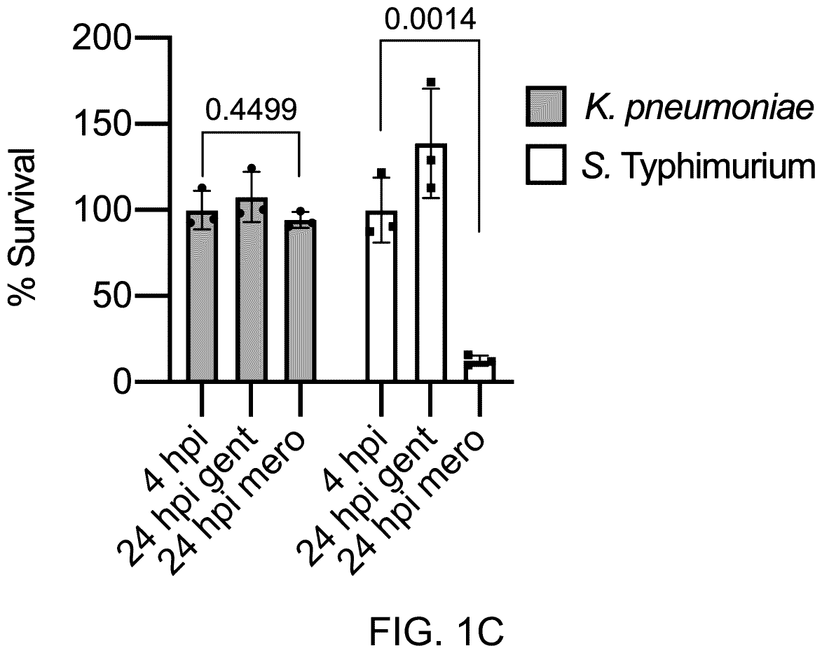

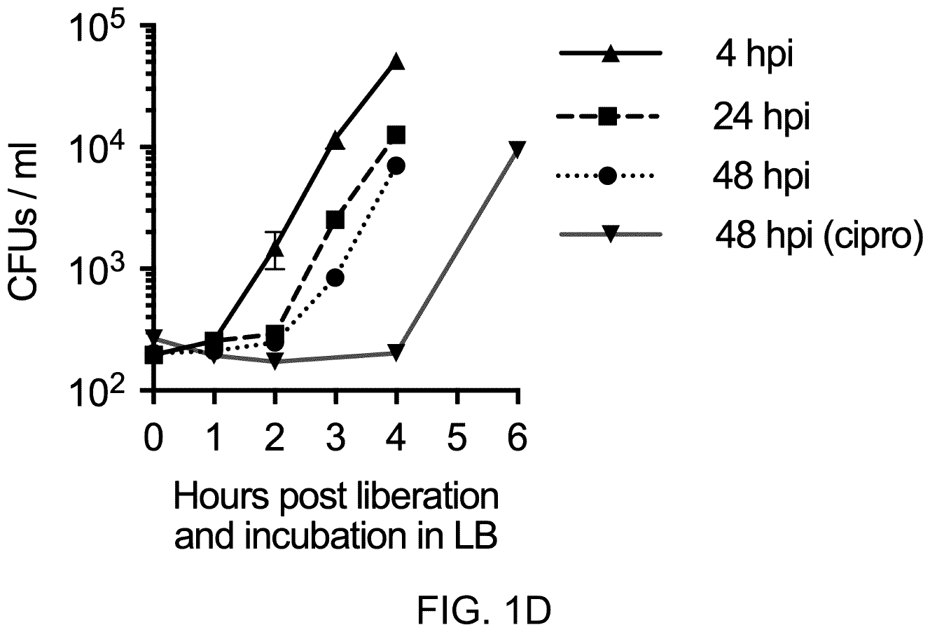

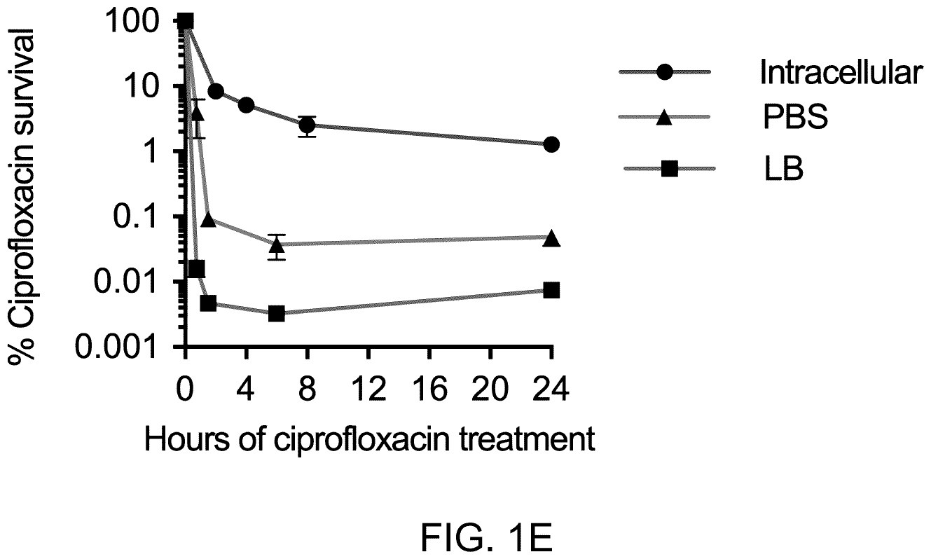

Multidrug-resistant Klebsiella pneumoniae are able to persist for several days in LAMP1 positive compartments of bladder epithelial cells and tolerate lethal concentrations of antibiotics of last resort¹⁰. In order to further characterize this phenomenon of intracellular antibiotic tolerance in K. pneumoniae, Applicants turned to an antibiotic susceptible urine isolate. As observed with multidrug-resistant K. pneumoniae¹⁰, the clinical antibiotic-susceptible urine isolate (RB120) was able to persist in bladder epithelial cells over the course of 48 hours (FIG. 1A) and was consistently detected in LAMP1-positive intracellular compartments (FIG. 1B). Treatment of the infected cells with the carbapenem antibiotic meropenem at (10 μg/ml, 150 fold of the minimal inhibitory concentration (MIC) determined in axenic medium) had no impact on the viability of intracellular K. pneumoniae but killed 87% of an intracellular Salmonella Typhimurium population after 20 hours of treatment (FIG. 1C), suggesting that meropenem was able to penetrate the host but was ineffective against intracellular K. pneumoniae, possibly due to the majority of the population being in a non-growing or slow-growing state, which is known to confer tolerance to most antibiotics, including beta lactam antibiotics⁵. In contrast to meropenem, the antibiotic ciprofloxacin is effective against non-replicating bacteria, except for bacteria that have entered an extremely dormant metabolic state⁵, in which most antibiotic targets are inactive. Ciprofloxacin (4 μg/ml, 100 fold of the MIC determined in axenic medium) killed 92% of the intracellular K. pneumoniae population within the first 2 hours of treatment, followed by slow killing over 22 hours, resulting in 99% killing activity after 24 hours of treatment (FIG. 1D). The biphasic killing kinetic with ciprofloxacin and the general tolerance to the beta lactam antibiotic meropenem suggested that the majority of the intracellular population persisted in the host in a non-growing but metabolically active (ciprofloxacin-susceptible) state and that a subpopulation persisted in a metabolically dormant, multidrug-tolerant⁵ state. Compared to axenic medium, ciprofloxacin tolerance occurred at more than 150 fold increased frequency in host cells, which could only be partly explained by nutrient-limiting conditions, as ciprofloxacin tolerance still occurred at more than 20 fold increased frequency in host cells, relative to phosphate buffered saline (FIG. 1D). In line with a dormant metabolic state^{5,11}, intracellular ciprofloxacin-tolerant K. pneumoniae that were liberated via host cell lysis and incubated in nutrient rich media, displayed a 2 fold extended growth lag phase compared to the bulk (untreated) intracellular population, which in turn displayed a 2 fold extended growth lag phase compared to bacteria that were liberated and grown shortly after infection (48 vs 4 hpi, FIG. 1E). Taken together, these results suggest that the intracellular environment of bladder epithelial cells gradually increases dormancy in K. pneumoniae, resulting in widespread carbapenem tolerance and increased frequency of a multidrug-tolerant subpopulation.

### Example 2—Crizotinib Enantiomers Emerge as Top Hits in a High-Throughput Screen of Repurposed Clinical Compounds Against Intracellular Carbapenem-Tolerant K. pneumoniae

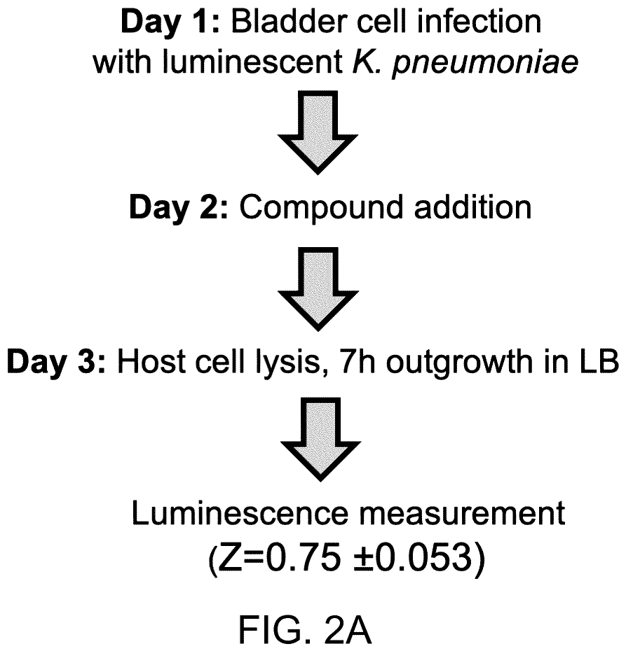

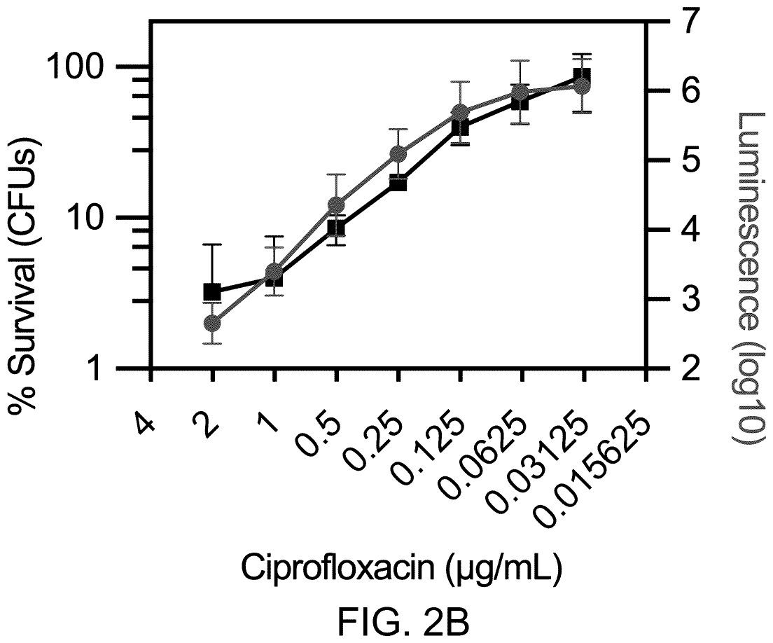

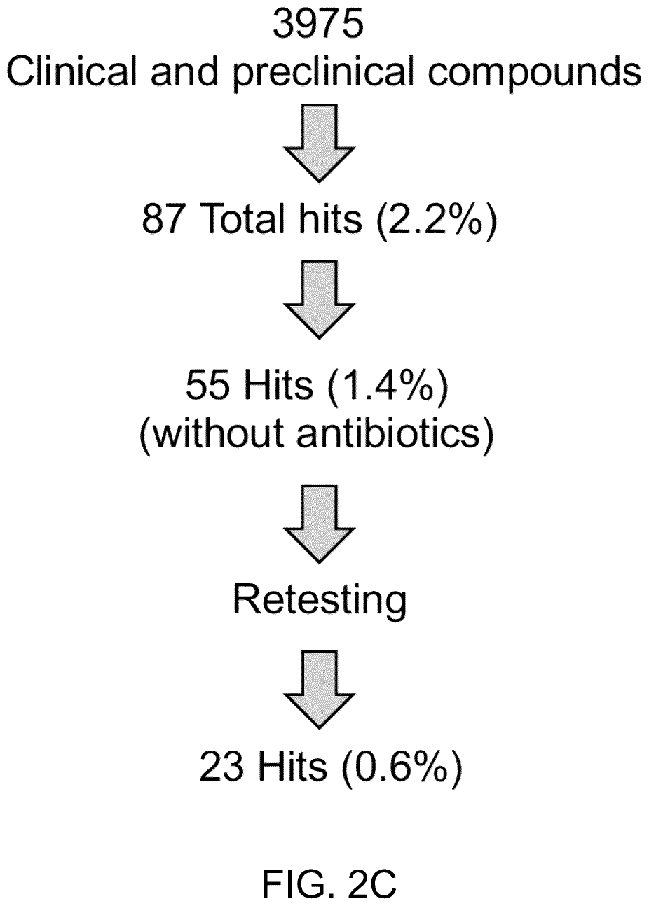

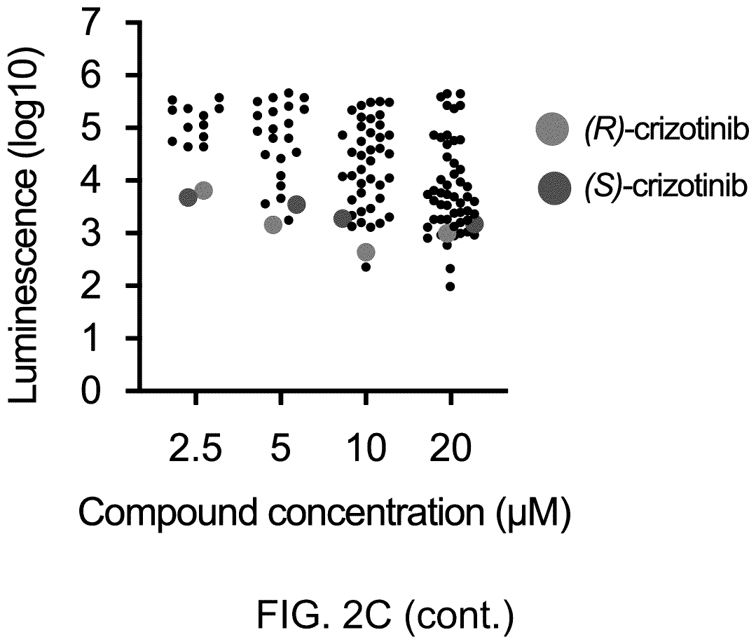

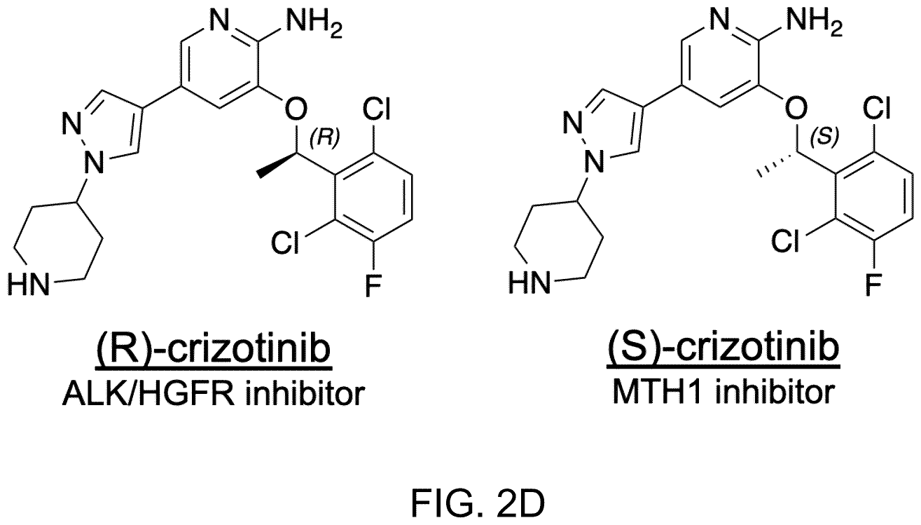

Because of the difficulty of effectively treating intracellular K. pneumoniae with common and last resort antibiotics, and the increasingly appreciated role of intracellular, antibiotic-tolerant bacterial persisters in chronic infections¹², Applicants explored the possibility of targeting the host with a repurposed library of clinical and preclinical compounds to identify compounds that may induce intracellular antibacterial activity in bladder cells infected with carbapenem-tolerant K. pneumoniae. First, with ciprofloxacin as positive control, Applicants developed a screening assay that enabled high-throughput screening of intracellular bacterial viability based on the level of outgrowth of luminescent K. pneumoniae from lysed host cells (FIG. 2A-2B). 3975 compounds were screened at a concentration of 5 μM, resulting in the identification of 87 hits (2%), which Applicants retested at concentrations ranging from 2.5 to 20 μM, confirming 23 of the 87 hits (0.6%) at the screening concentration of 5 μM (FIG. 2C). Remarkably, the two crizotinib enantiomers (R)- and (S)-crizotinib, which are known anticancer compounds with different mechanisms of action¹³, consistently emerged among the top hits at all tested concentrations. (R)-crizotinib (marketed by Pfizer as Xalkori) is a tyrosine kinase receptor inhibitor, which has been FDA approved for ALK or ROS1 positive non-small-cell lung cancer and ALK positive anaplastic large cell lymphoma¹⁴, while (S)-crizotinib is a preclinical anticancer compound that inhibits MTH1, a nucleotide sanitizing enzyme which prevents the genomic integration of oxidized nucleotides that would otherwise lead to lethal mutations¹³ (FIG. 1D). Since oxidative stress occurs at higher levels in fast growing cancer cells, MTH1 has been suggested to be a promising anticancer target which specifically targets cancer cells¹³. (S)-crizotinib has been shown to inhibit MTHI at low nanomolar concentrations (72 nM) in cell free assays and inhibits colony formation of cancer cells at low micromolar concentrations¹³. However, MTH1 inhibitors, including (S)-crizotinib, have displayed inconsistent or MTH1 independent cytotoxic effects, casting doubts on the concept of targeting MTH1 as anticancer therapy¹⁵.

### Example 3—(S)-Crizotinib Induces Rapid Intracellular Antimicrobial Activity in Infected Epithelial Cells

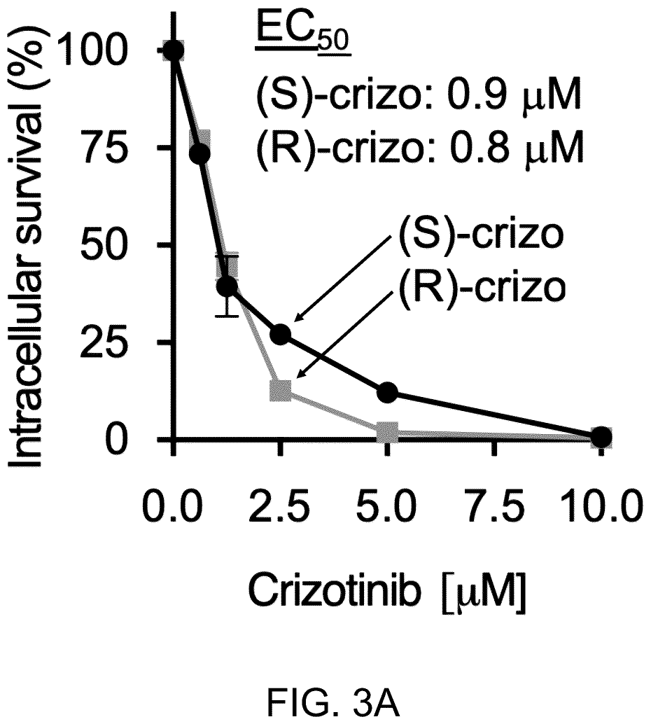

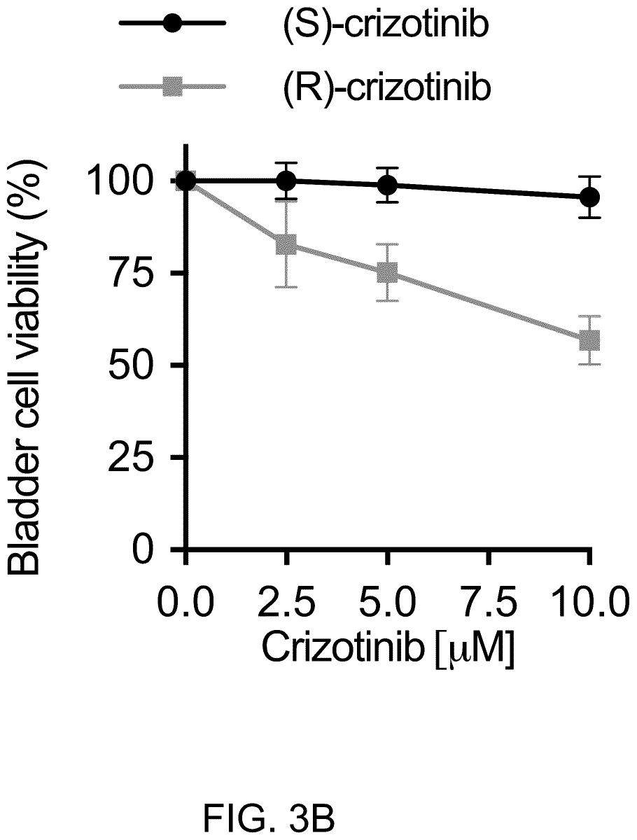

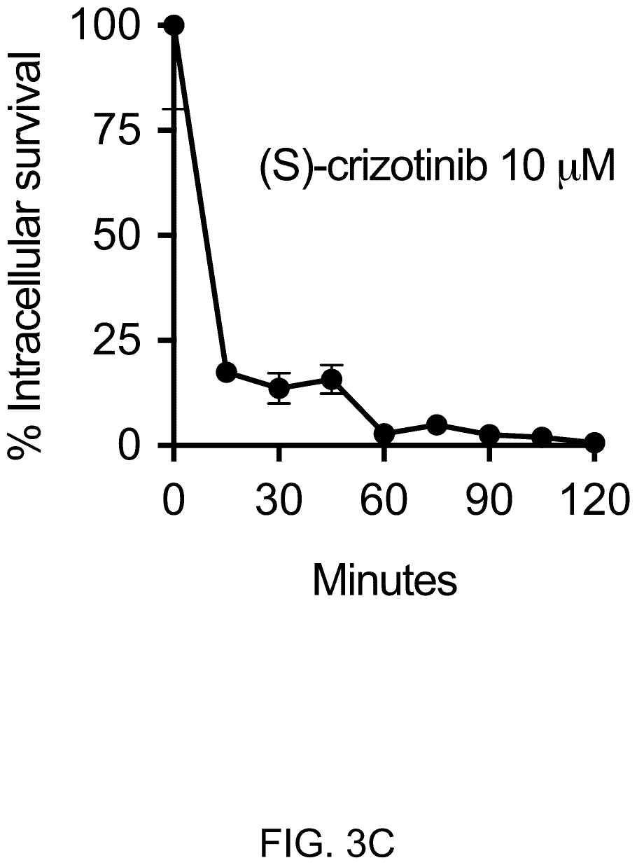

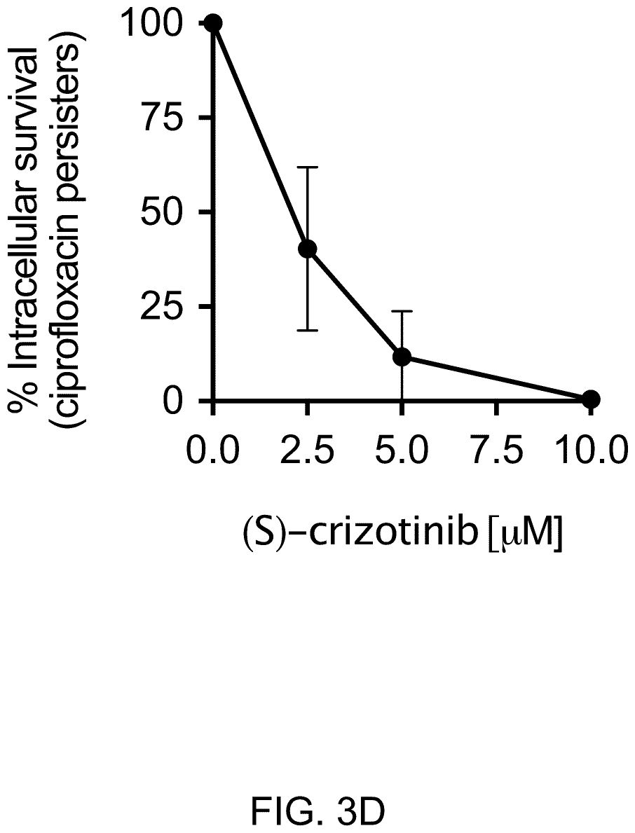

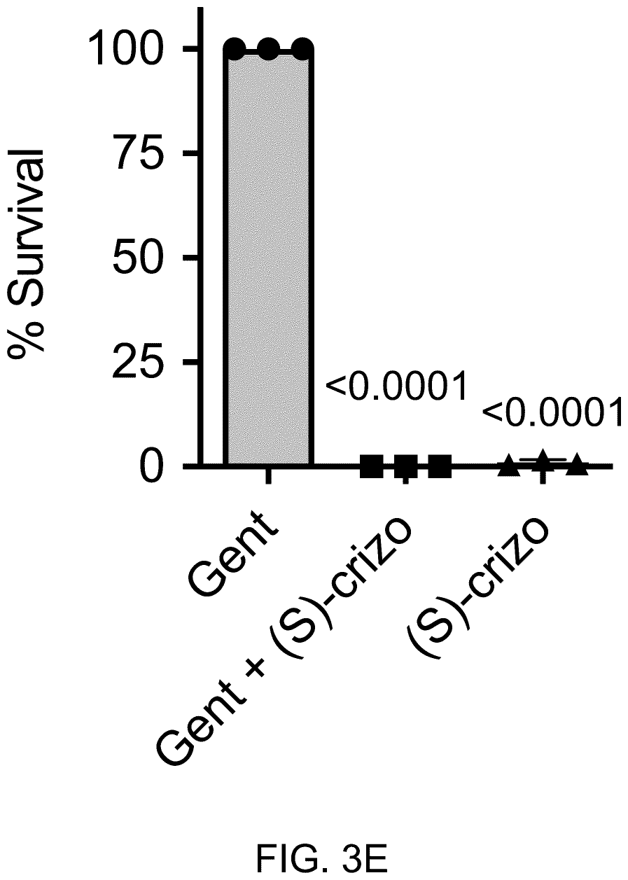

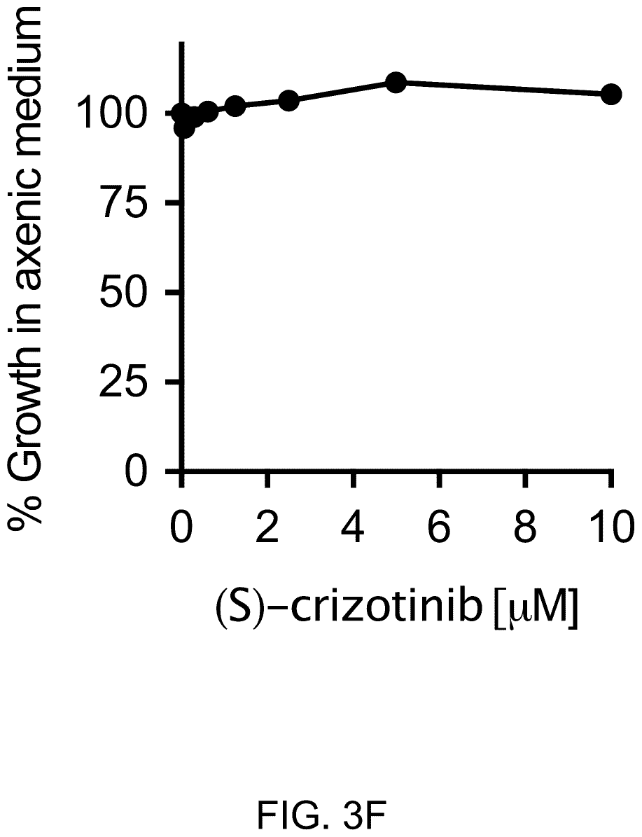

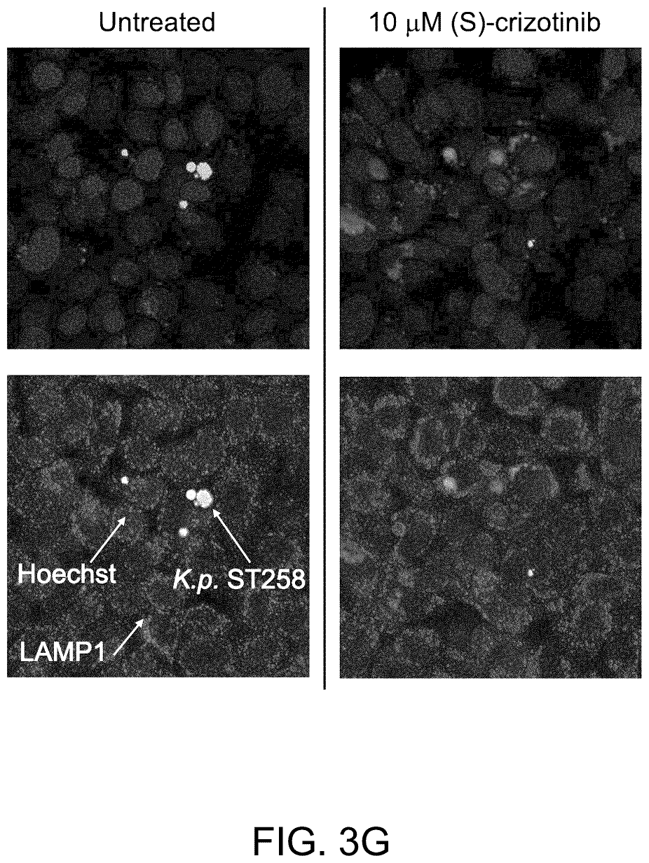

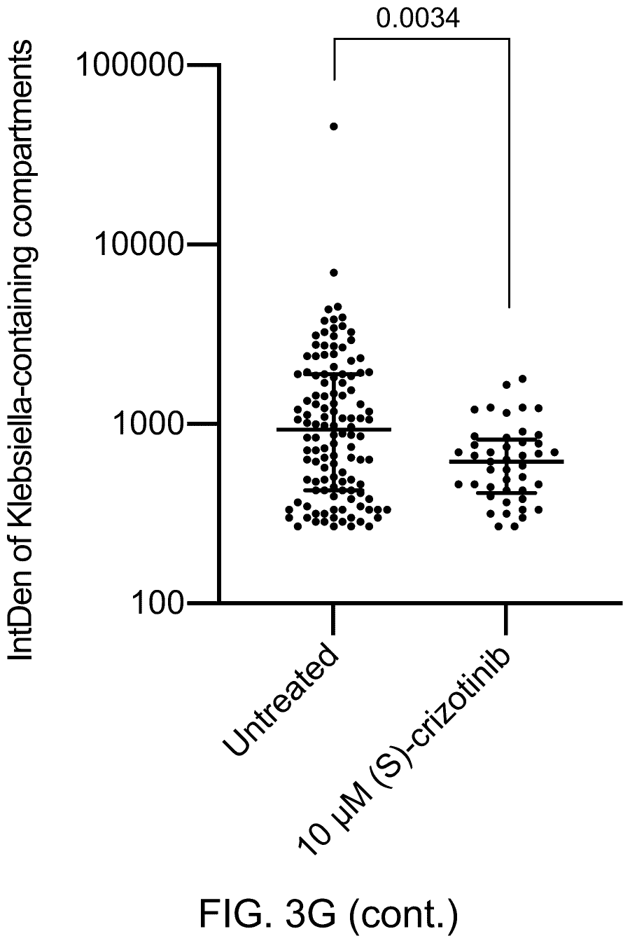

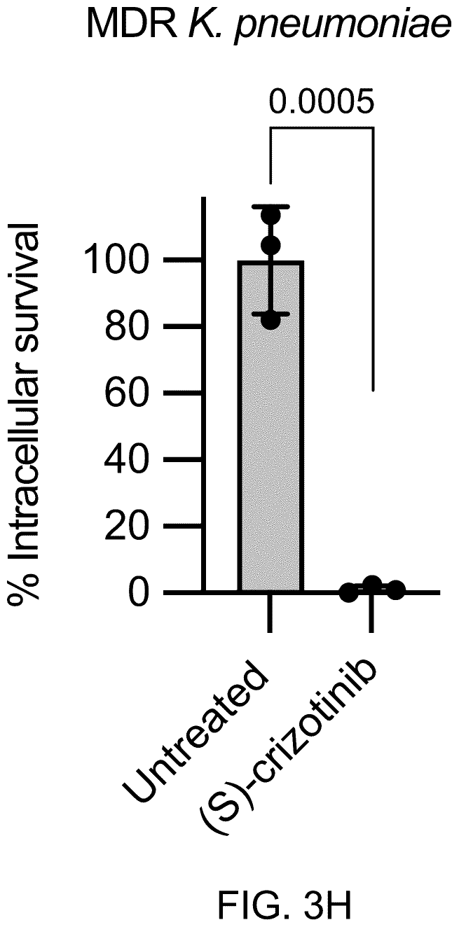

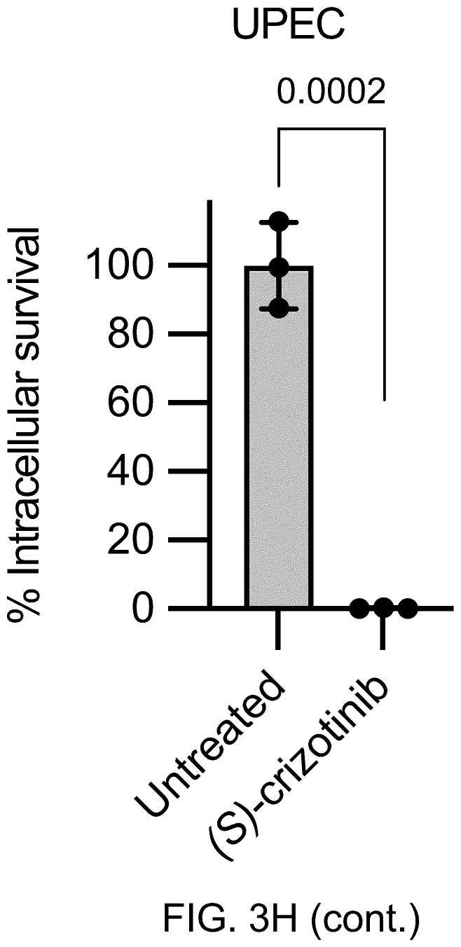

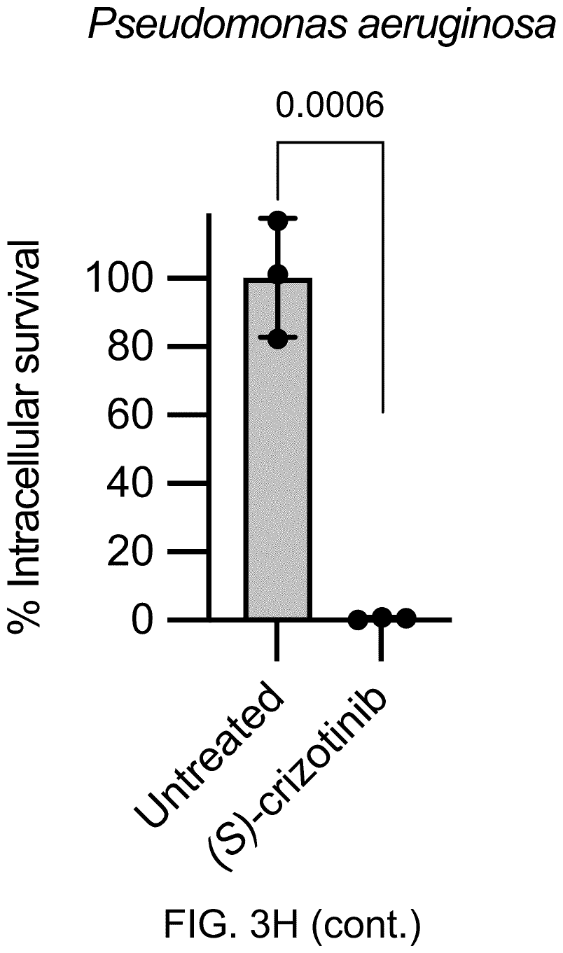

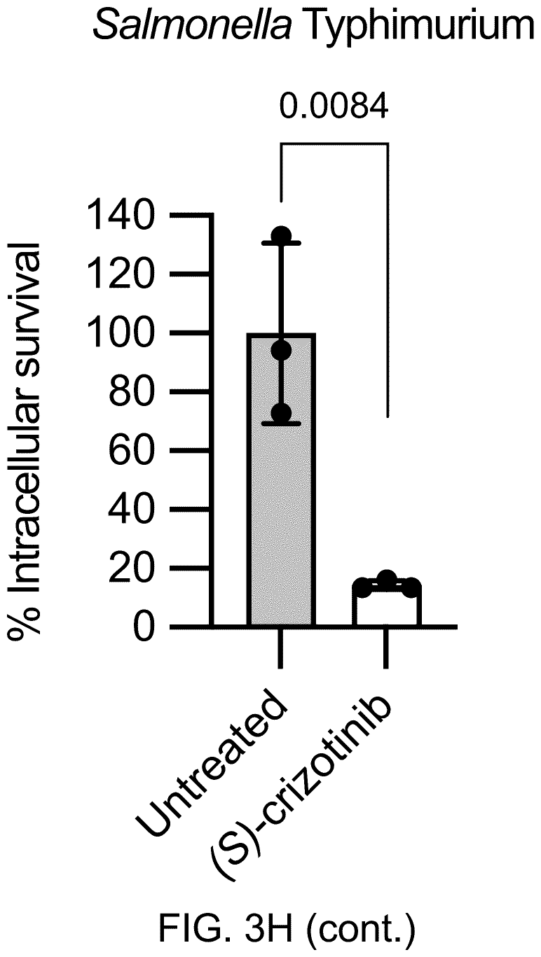

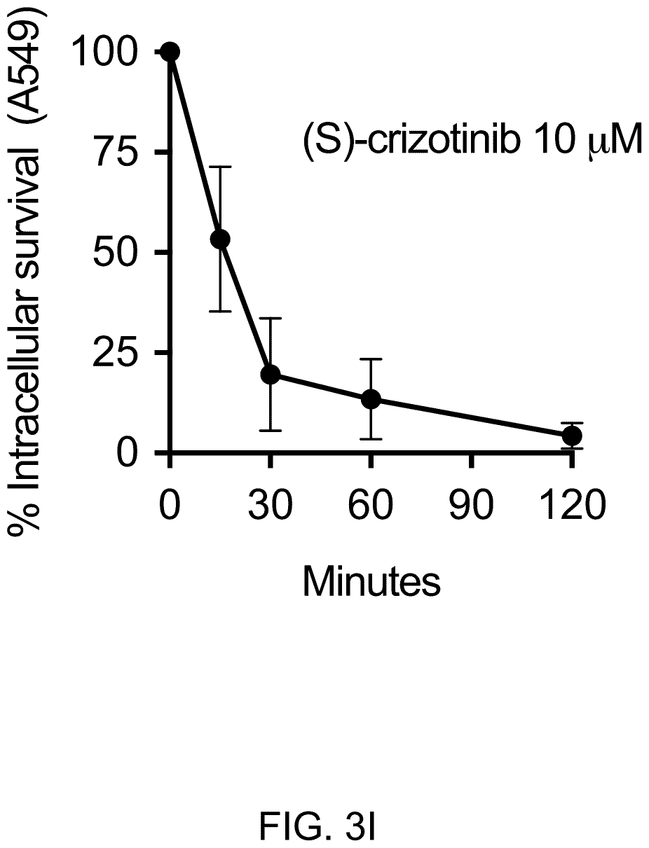

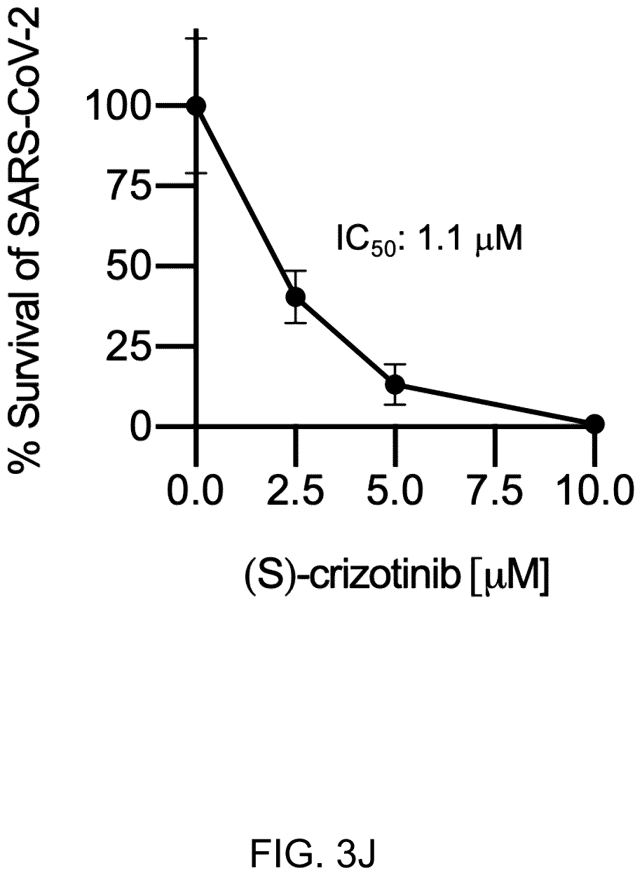

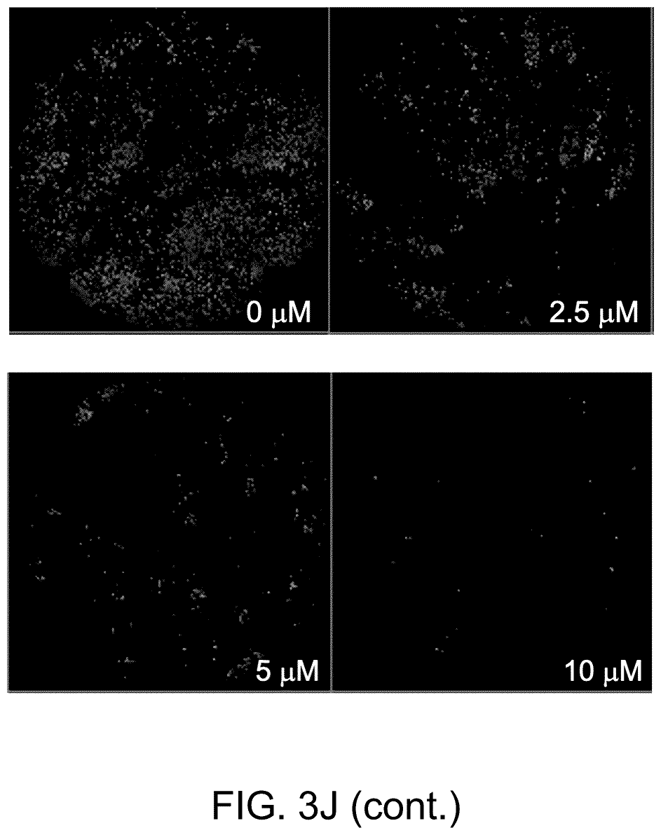

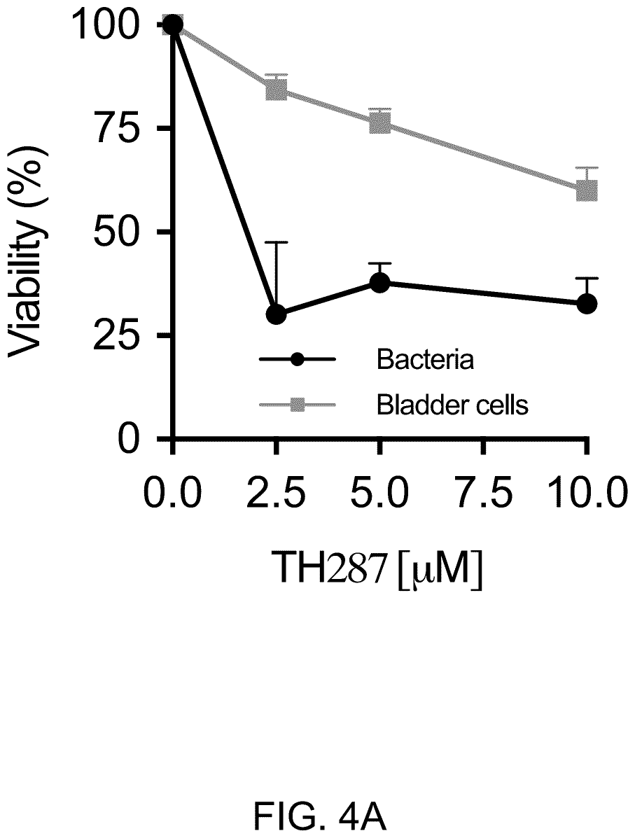

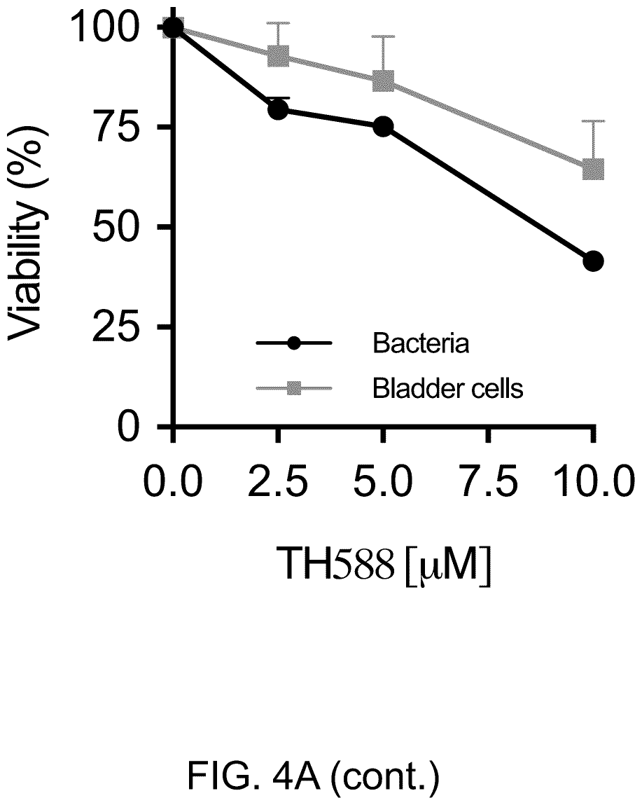

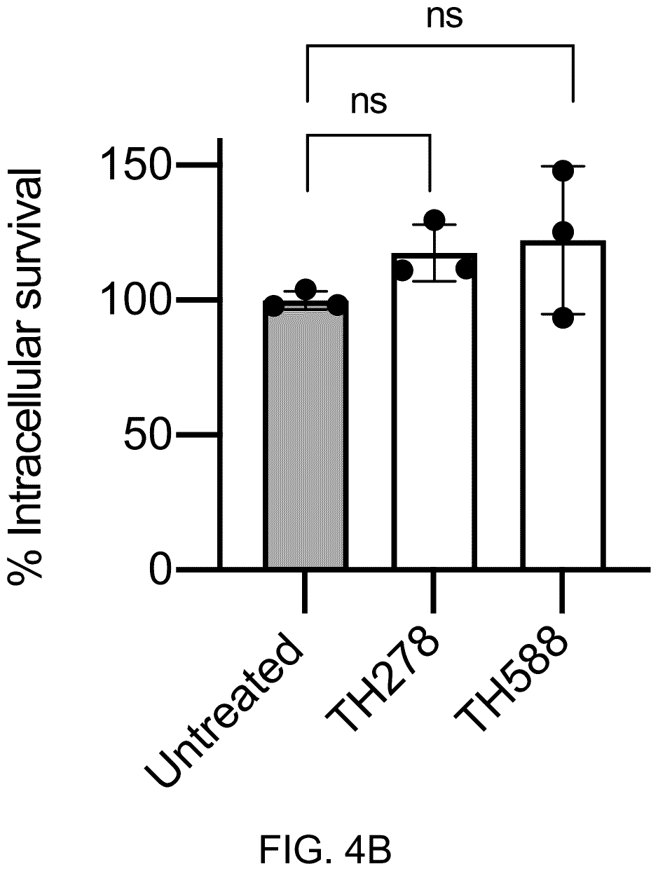

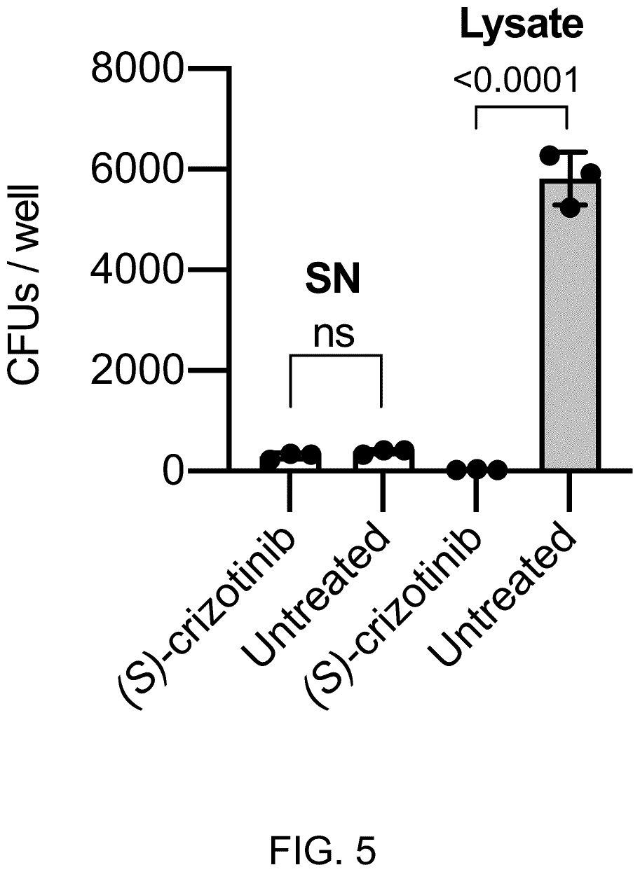

(R)- and (S)-crizotinib both displayed dose-dependent killing activity against intracellular K. pneumoniae after 24 hours of treatment, killing 99% of the population at a concentration of 10 μM (EC50: 1.1/1.7 μM, FIG. 3A). While (S)-crizotinib did not display cytotoxicity against bladder epithelial cells, (R)-crizotinib displayed host cell cytotoxicity in a dose-dependent manner (FIG. 3B), consistent with its documented mode of action of inhibiting multiple essential tyrosine kinase receptors^{13,14}. Strikingly, (S)-crizotinib (10 μM) displayed rapid intracellular killing activity, killing 82% of the intracellular population within 15 minutes, with 99% of the population killed after 2 hours of treatment (FIG. 3C). Importantly, the fact that (S)-crizotinib and (R)-crizotinib were able to induce rapid intracellular antibacterial activity in host cells, and the lack of cytotoxicity in (S)-crizotinib treated bladder epithelial cells, excluded the possibility that the anti-infective property of (S)-crizotinib was due to accumulation of lethal mutations via inhibition of MTH1, which was supported by the observation that other MTH1 inhibitors (TH287 and TH588) did not display intracellular antibacterial activity after 4 hours of treatment (10 μM), and only led to modest antibacterial activity after 24 hours of treatment at a concentration of 10 μM, which correlated with host cell toxicity (FIG. 4A-4B). Moreover, Applicants found that (S)-crizotinib displayed intracellular, dose-dependent antibacterial activity against ciprofloxacin persisters (FIG. 3D), suggesting that most of the intracellular bacteria that survived (S)-crizotinib treatment were not in a metabolically dormant, multidrug-tolerant state⁵. Removal of gentamicin from the culture medium, which was used to prevent extracellular bacterial growth in our infection assays, had no impact on the potent antibacterial activity of (S)-crizotinib (FIG. 3E), excluding a potential contribution of the antibiotic to the observed antibacterial activity. (S)-crizotinib did not display antibacterial activity against extracellular K. pneumoniae grown in axenic medium, suggesting that its antibacterial effect was induced in the host (FIG. 3F). To better characterize the impact of (S)-crizotinib on Klebsiella-containing compartments Applicants infected bladder epithelial cells with a previously characterized multidrug-resistant, capsule-deficient K. pneumoniae mutant, which infects cells at higher frequency and has a high tendency to grow in LAMP1 compartments¹⁰. (S)-crizotinib treatment of infected bladder epithelial cells for 24 hours resulted in a 63% reduction of detected Klebsiella containing compartments and a 33% reduction in the median fluorescence intensity of mNeon-expressing Klebsiella in LAMP1 containing compartments (FIG. 3G). In the absence of gentamicin, (S)-crizotinib treatment of infected bladder epithelial cells did not result in a significant increase of extracellular bacteria (FIG. 5), indicating that the antimicrobial effect was not due to exocytosis¹⁶ of Klebsiella-containing compartments. The antibacterial effect of (S)-crizotinib was not restricted to a particular intracellular pathogen or cell line, as Applicants observed antibacterial activity in bladder epithelial cells infected with multidrug-resistant K. pneumoniae (ST258), uropathogenic Escherichia coli (UPEC), Pseudomonas aeruginosa and Salmonella Typhimurium (FIG. 3H) and in infected lung epithelial cells (FIG. 3I). Given the intracellular anti-infective property of (S)-crizotinib Applicants decided to further test its efficacy against viral infection. As observed with intracellular bacteria, Applicants found that (S)-crizotinib also induced antiviral activity in epithelial cells infected with SARS-COV-2 in a dose dependent manner (FIG. 3J), revealing broad spectrum anti-infective properties of (S)-crizotinib against intracellular pathogens, including viruses. Taken together, these results confirm the remarkable ability of (S)-crizotinib to induce broad spectrum anti-infective activity in host cells and its efficacy against intracellular pathogens that can otherwise not effectively be treated with anti-infectives.

### Example 4—(S)-Crizotinib Displays Antimicrobial Efficacy in a Mouse Model of Latent Urinary Tract Infection

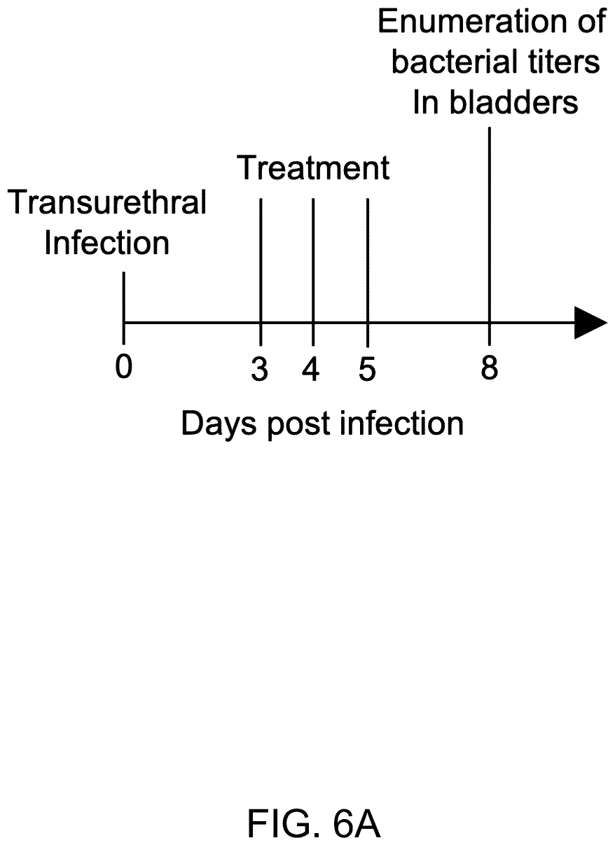

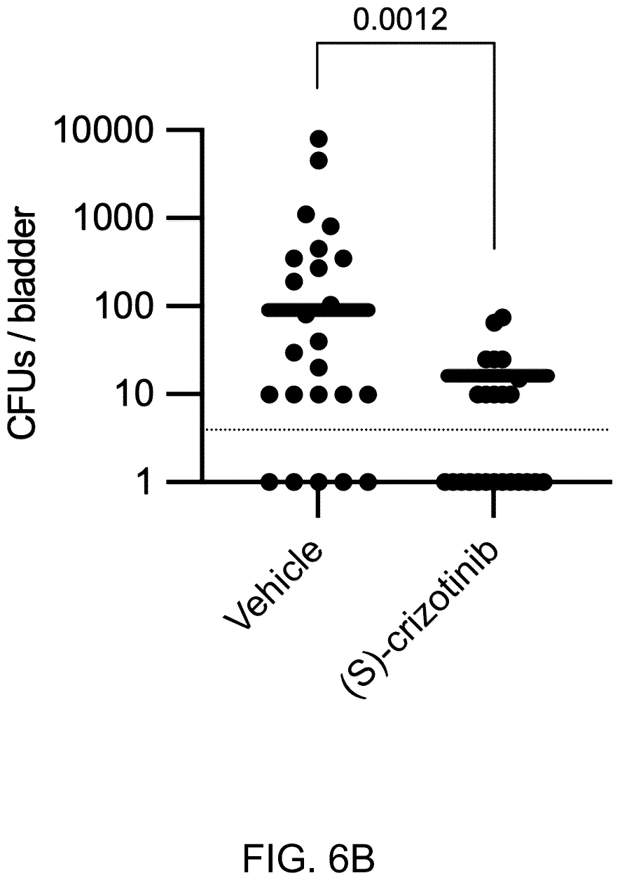

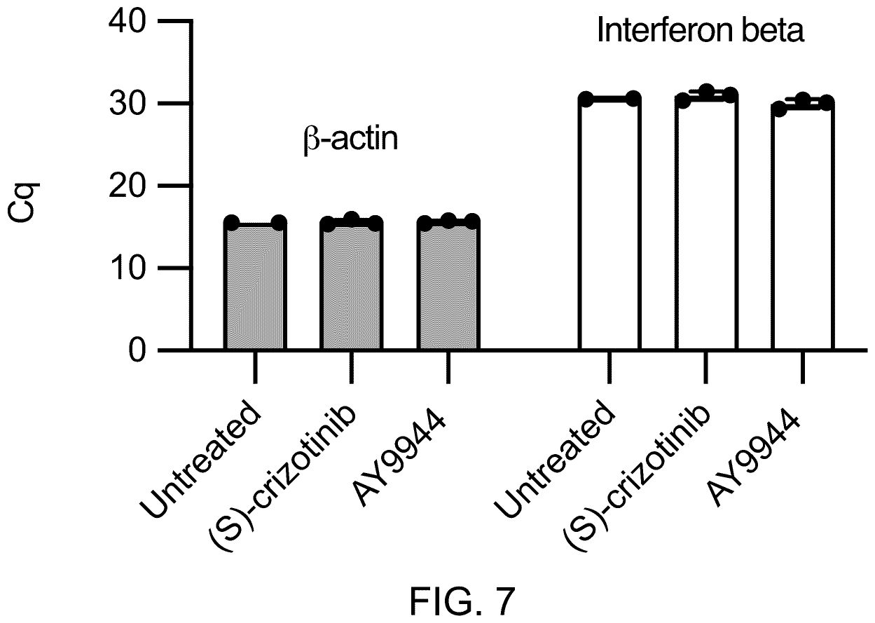

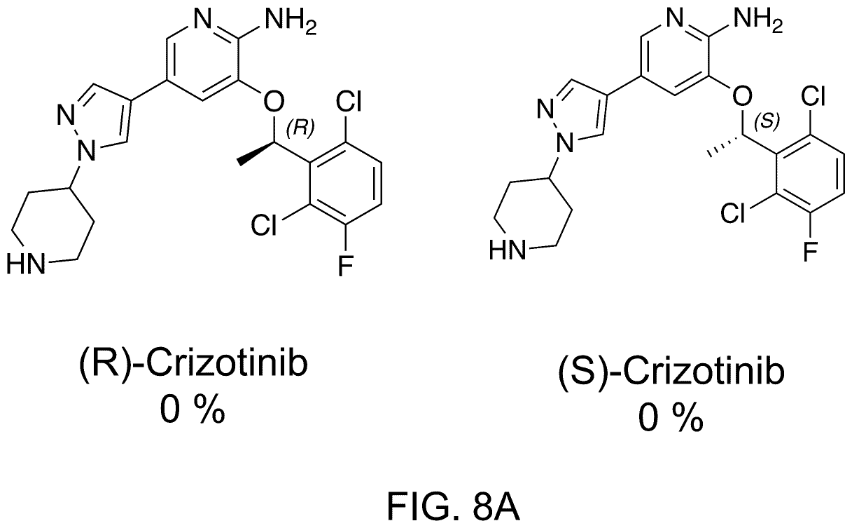

(S)-crizotinib has been previously shown to suppress tumor growth in mice¹³ and is structurally related to (R)-crizotinib, which is used in the clinic to treat lung cancer in patients¹⁴. Intracellular quiescent reservoirs have been observed in mouse models of chronic and latent infection with uropathogenic Escherichia coli and K. pneumoniae, and are likely to be a cause of recurring urinary tract infection¹⁷. Applicants tested the in vivo efficacy of (S)-crizotinib in a mouse model of latent urinary tract infection^{17,18}. Female BALB/c mice infected with a capsule-deficient multidrug-resistant ST258 mutant, which was recently shown to persist in the bladder in a mouse model of urinary tract infection¹⁰. After 3 days of infection the mice were treated subcutaneously with 25 mg/kg (S)-crizotinib for 3 consecutive days and sacrificed after 8 days of infection to enumerate bacterial titers in the bladder (FIG. 6A). Applicants found that (S)-crizotinib treatment of mouse UTIs substantially decreased bacterial numbers in mouse bladders, compared to vehicle treated mice (FIG. 6B). Thus, the anti-infective effect of (S)-crizotinib that Applicants had observed in cultured epithelial cells had also occurred in vivo, suggesting that (S)-crizotinib may have potential to be repurposed for the treatment of latent bacterial infections to prevent recurring infections, such as urinary tract infections.

### Example 5—Structure-Activity Relationship (SAR) Analysis

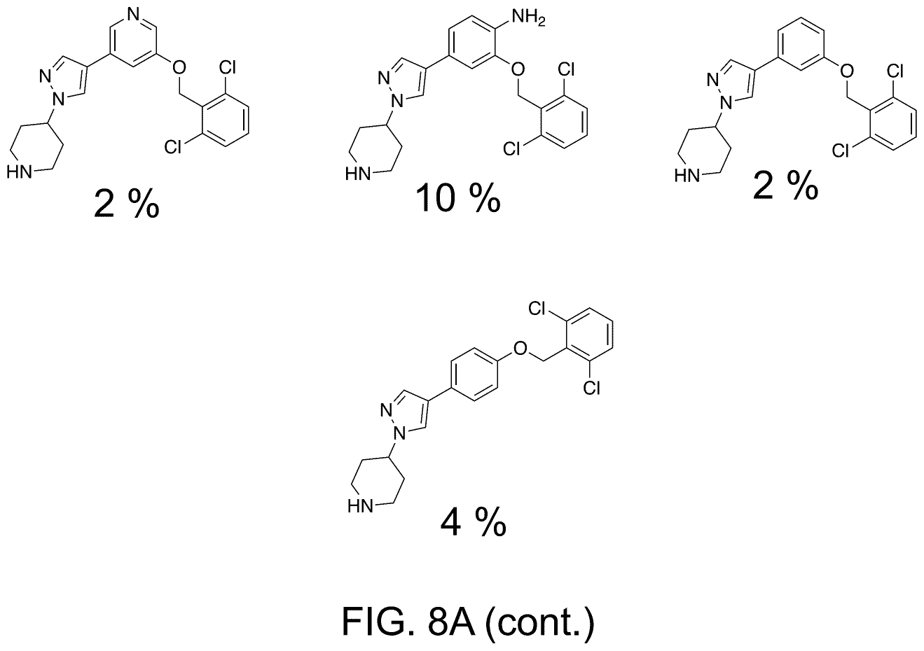

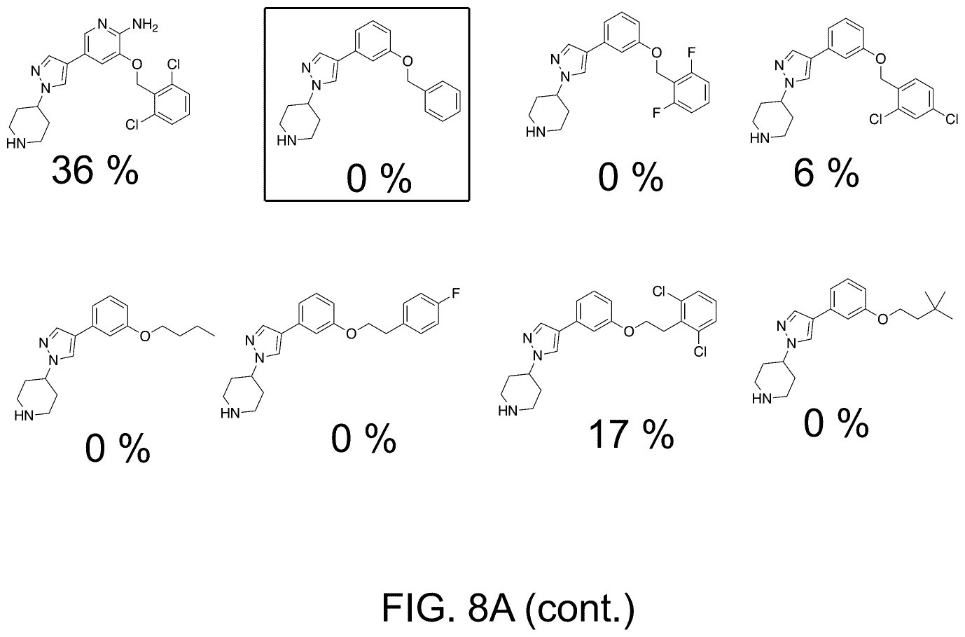

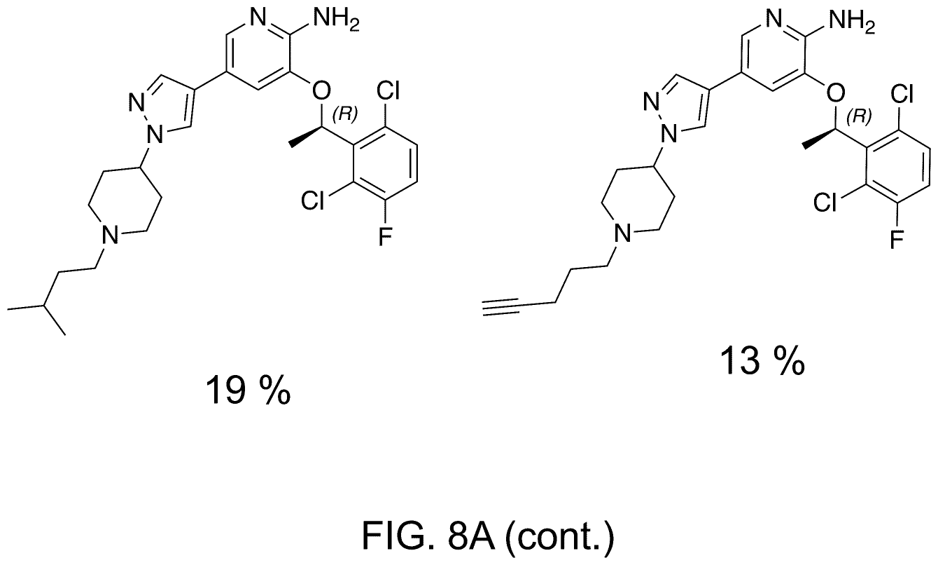

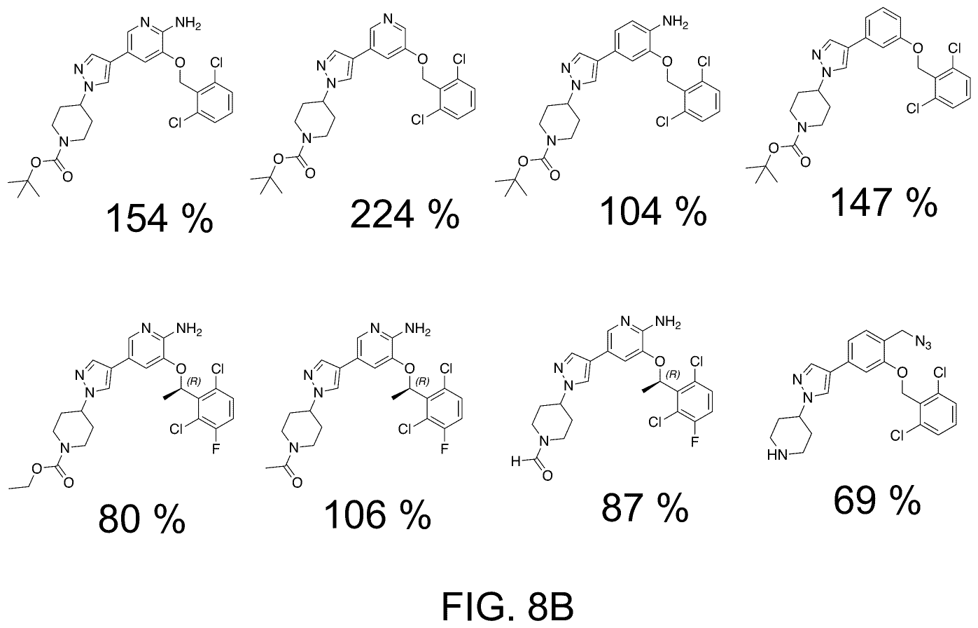

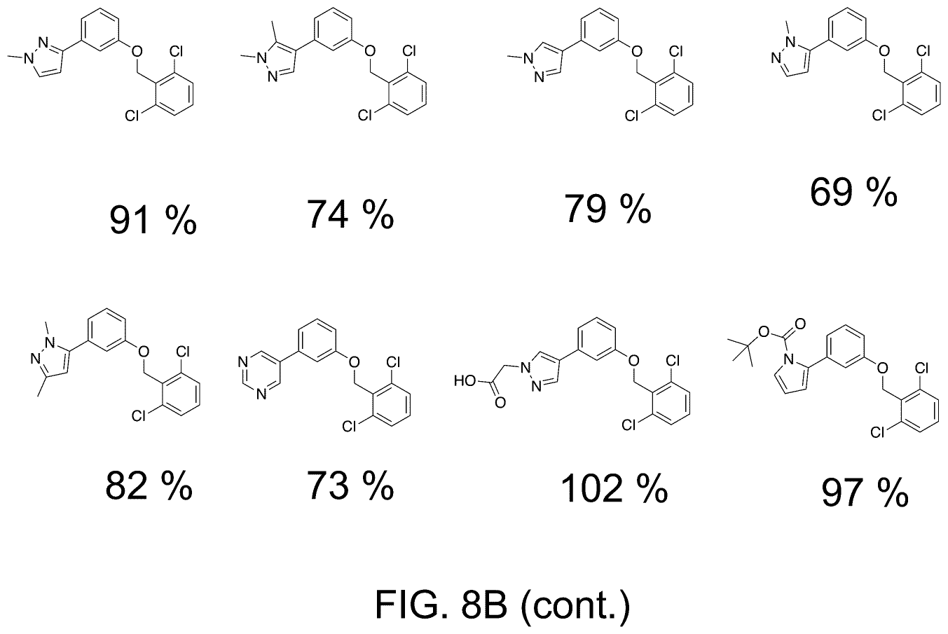

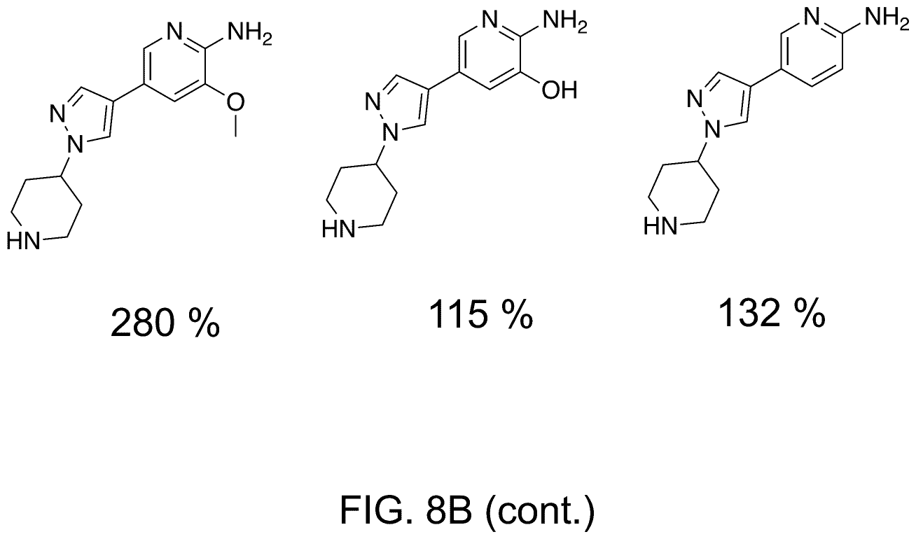

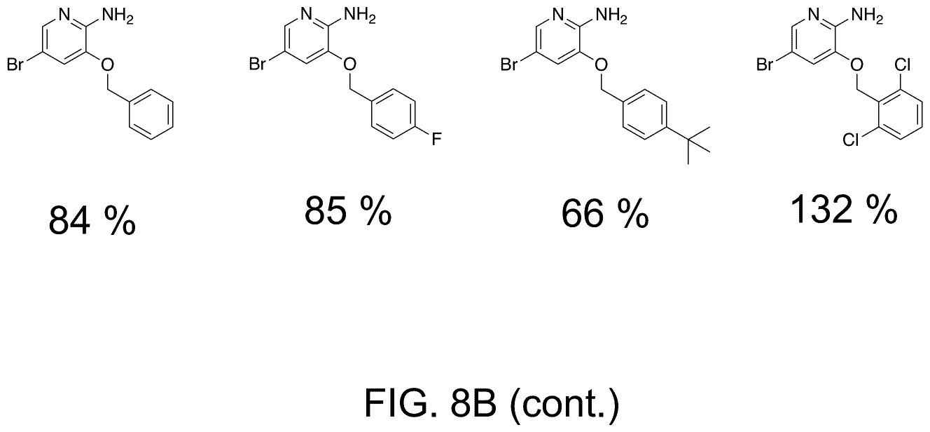

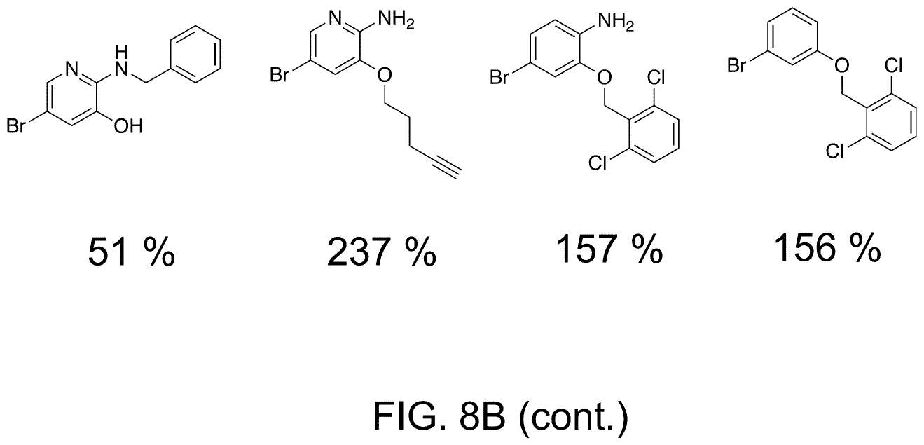

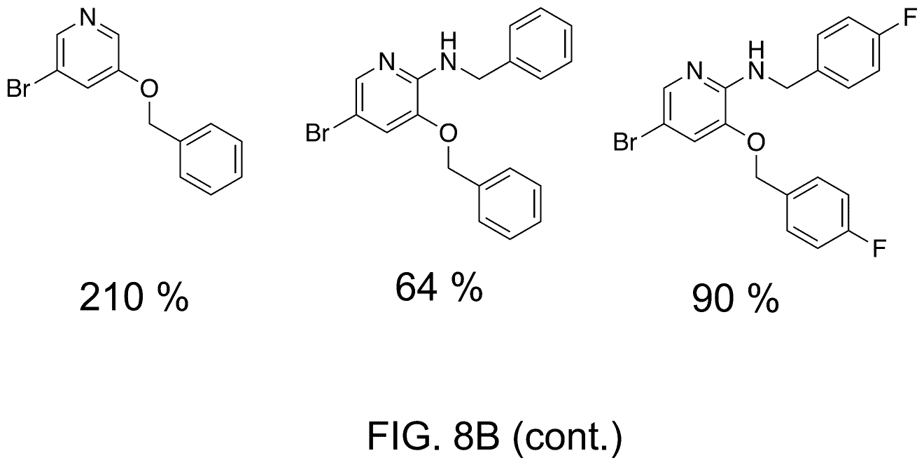

Intracellular survival of Klebsiella pneumoniae (RB120) after four-hour treatment with 10 μM crizotinib analogs predicted structures is shown in FIGS. 8A-8B. The mean percentage of recovered bacteria from treated bladder epithelial cells relative to untreated DMSO control from n=3 biological replicates in 96 well plates is shown. Examplary structures shown in FIG. 8A displayed similar or equivalent antimicrobial activity as (R)-crizotinib and (S)-crizotinib. The exemplary structures shown in FIG. 8B displayed no or substantially reduced antibacterial activity compared to (R)-crizotinib and (S)-crizotinib.

### Example 6—Analysis of the Antimicrobial Target of (S)-Crizotinib

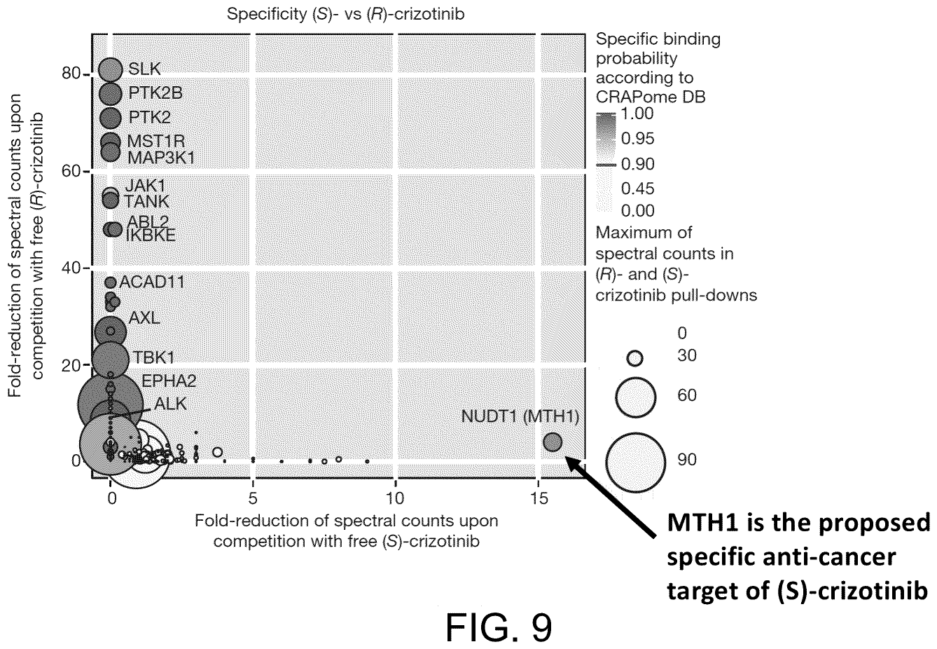

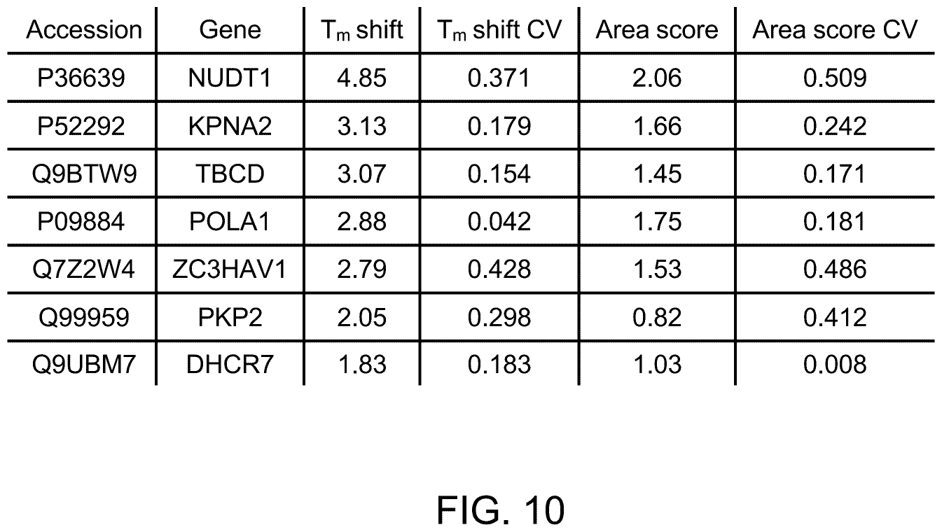

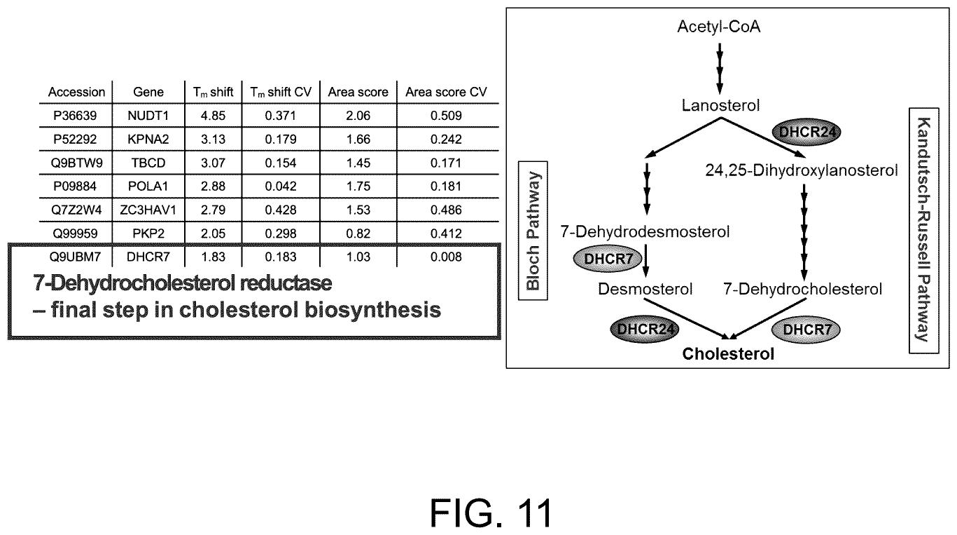

Several antimicrobial targets for (S)-crizotinib were investigated, as shown in FIGS. 9-12. Further, the 7-dehydrocholesterol reductase (DHCR7) inhibitor AY 9944, structure below:

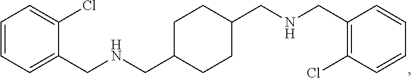 [SMILES: Clc1ccccc1CNCC1CCC(CNCc2ccccc2Cl)CC1]

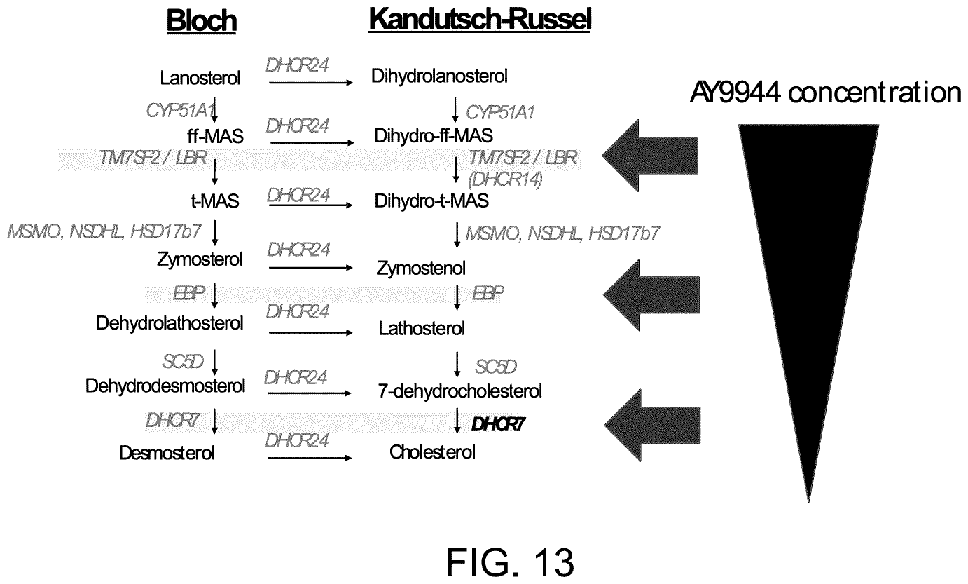

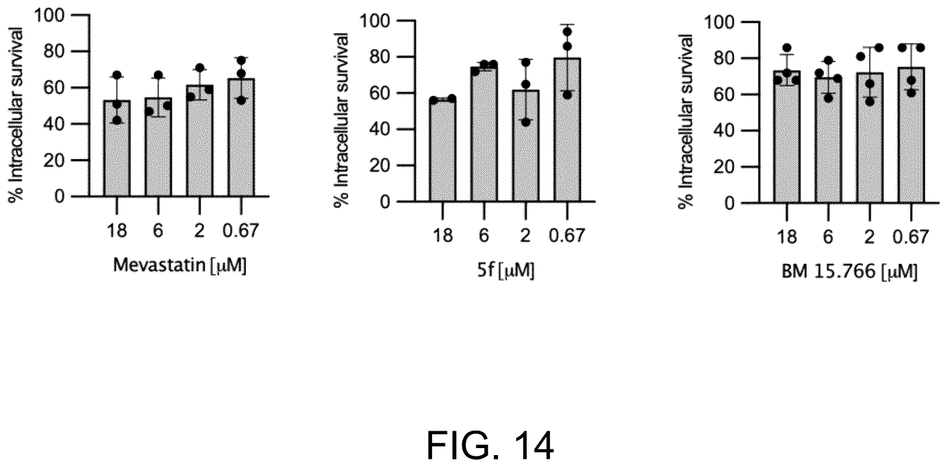

was found to mirror the antimicrobial activity of (S)-crizotinib, as shown in FIG. 13. Finally, the antimicrobial activity of both (S)-crizotinib and AY 9944 appear to depend on a cholesterol intermediate in the post-lanosterol cholesterol biosynthesis pathway, as shown in FIG. 14.

### Example 7—A Structure Activity Relationship Investigation Suggests that a Secondary Target of Crizotinib is Involved in the Induction of Antimicrobial Activity

(S)-crizotinib and (R)-crizotinib both have similar antimicrobial efficacy but have different modes of action, suggesting that the primary targets of (S)-crizotinib and (R)-crizotinib are not involved in the observed antimicrobial activity, in line with our detailed structure activity relationship (SAR) investigation of crizotinib, which showed that antimicrobial activity did not depend on the chirality of crizotinib or on the central aminopyridine ring. The aminopyridine ring in crizotinib is required for binding to the hinge region of receptor kinases^{19,20} and is likely involved in MTH1 binding as shown with the aminopyrimidine ring of other MTH1 inhibitors²¹. The aminopyridine ring also appears to be involved in the inhibition of the bacterial CTP synthase PyrG, involved in bacterial pyrimidine metabolism, resulting in inhibition of DNA synthesis and growth inhibition of Gram-positive bacteria at a concentration >20 uM²². Thus, the dispensability of the aminopyridine ring in the induction of intracellular antimicrobial activity in host cells suggests that antimicrobial activity is not mediated by the inhibition of tyrosine receptor kinases or MTH1 or PyrG, in line with the modest antimicrobial activity seen with the MTH1 inhibitor TH588, which did not induce intracellular antibacterial activity after 4 hours of treatment (10 μM), and only induced modest antibacterial activity that correlated with host cell toxicity, after 24 hours of treatment (10 μM) (FIGS. 15A-15B). Of note, a recent study investigating additional targets of (S)-crizotinib with thermal stability profiling found that (S)-crizotinib bound to nucleotide binding enzymes (DNA polymerase alpha 1, zinc finger antiviral protein ZC3HAV1)²³, possibly due to the similarity of the aminopyridine ring of crizotinib with the pyrimidine ring of nucleotides, including TLR7 agonists, which mimic nucleotides with pyrimidine ring structures, and can also bind to MTH1²⁴. However, the TLR7 agonist imiquimod only displayed moderate anti-infective activity (FIG. 16). Thus, nucleotide binding proteins did not seem to be involved in the rapid induction of crizotinib-mediated intracellular antimicrobial activity in host cells.

### Example 8—Crizotinib Inhibits Multiple Dehydrocholesterol Reductases and CYP51A1

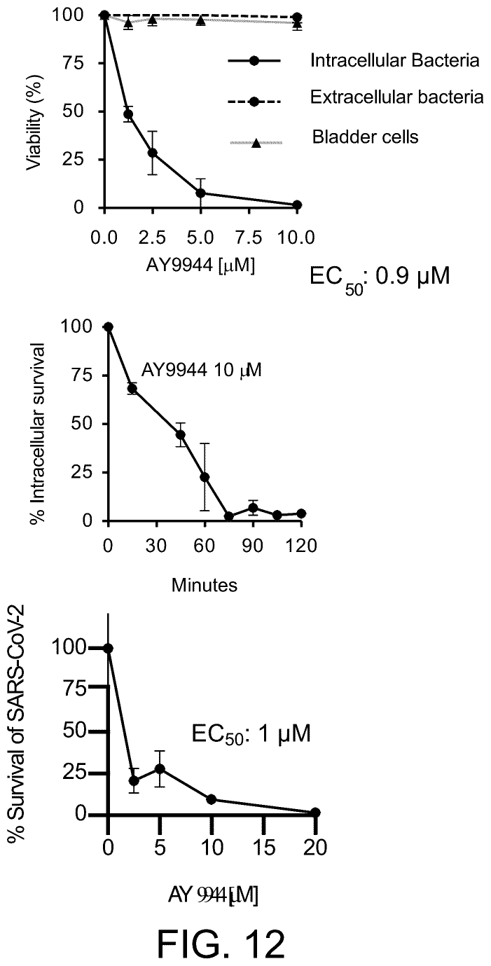

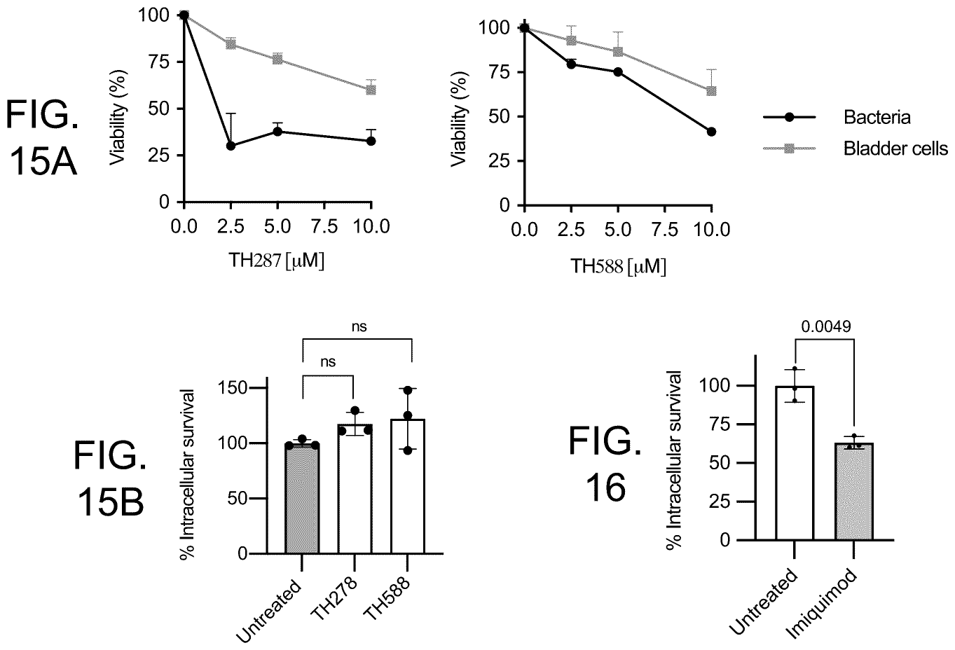

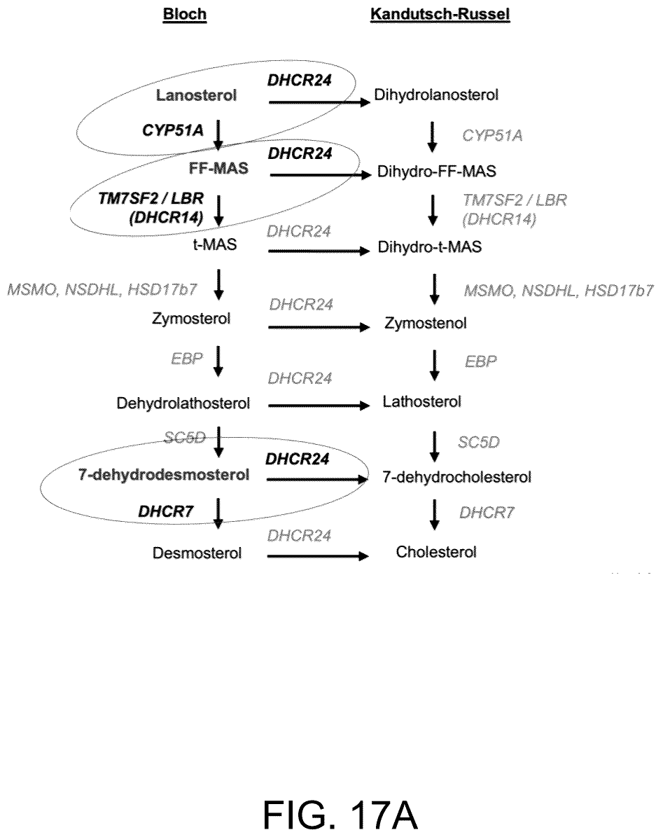

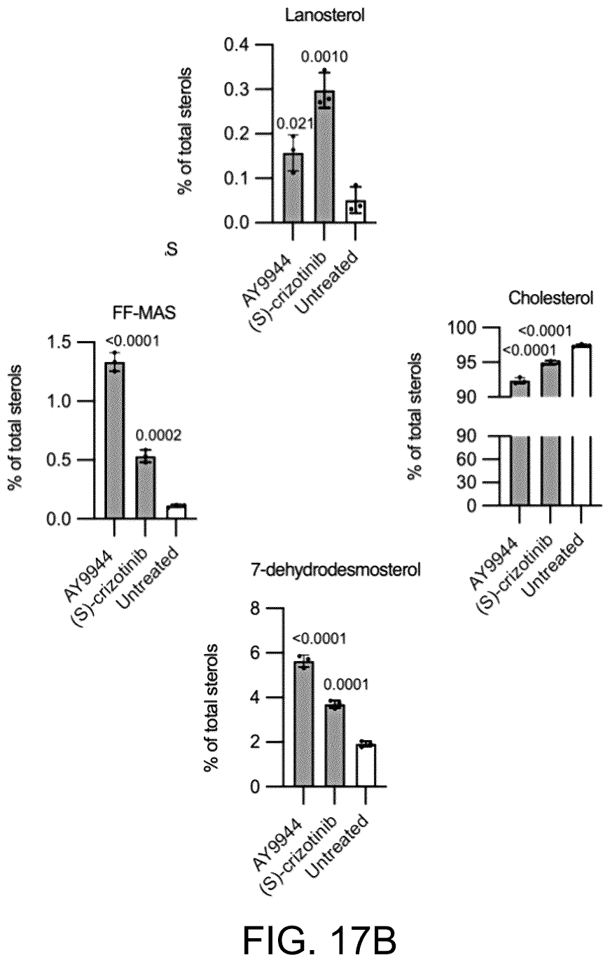

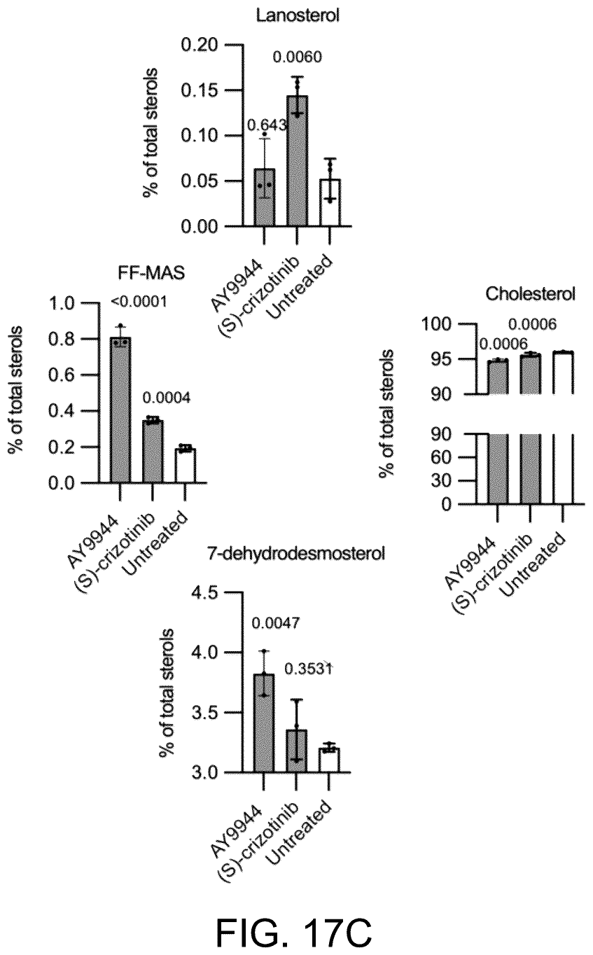

Aside from nucleotide binding proteins, several other proteins were recently identified as binding partners of (S)-crizotinib via thermal stability profiling, including karyopherin alpha 2 (KPNA2), tubulin specific chaperon D (TBCD), plakophilin 2 (PKP2) and 7-dehydrocholesterol reductase (DHCR7)²³. Intriguingly, the DHCR7 inhibitor AY 9944 also came up as a hit in a chemical screen. AY 9944 induced similar intracellular antimicrobial activity as (S)-crizotinib in bladder epithelial cells, displaying dose-dependent antimicrobial activity over 24 hours (EC50: 0.94 μM) and no host cell toxicity or antimicrobial activity in axenic medium (FIG. 12). Rapid antimicrobial activity was also observed at a concentration of 10 uM, which killed approx. 80% of the population in 60 minutes (FIG. 12), and similar antiviral activity as (S)-crizotinib was observed in SARS-COV-2 infections (EC50: 1 uM) (FIG. 12). Importantly, neither the more specific DHCR7 inhibitors BM 15.766 and 5f²⁵ nor the inhibition of the first step of cholesterol biosynthesis, HMG-CoA reductase, via mevastatin induced dose-dependent antimicrobial activity (FIG. 13), suggesting that the inhibition of DHCR7 or the depletion of cholesterol per se was not inducing dose-dependent intracellular antimicrobial activity. Since AY 9944 is known to bind to enzymes that are upstream of DHCR7 in the post-lanosterol cholesterol biosynthesis pathway (EBP, DHCR14²⁶, FIG. 14) (at micromolar concentrations), the impact on infection of other available inhibitors of the post-lanosterol cholesterol biosynthesis pathway (FIG. 17A) was tested.

LC-MS based lipidomics revealed that the treatment of infected and non-infected host cells with 10 μM AY9944 or 10 μM (S)-crizotinib led to the significant accumulation of lanosterol, FF-MAS and 7-dehydrodesmosterol (FIG. 17C) after 24 hours of treatment, which could already be seen after 2 hours of treatment (FIG. 17B). Among detectable sterols, lanosterol was the most abundant sterol intermediate after 2 or 24 hour (S)-crizotinib treatment (2.7/5.8 fold increased, respectively), followed by FF-MAS (1.8/4.8 fold increased) and dehydrodesmosterol (ns/1.9 fold increased). In the case of AY 9944 treatment, the most abundant sterol intermediates after 2 hour or 24 hour treatment were FF-MAS (4.2/11.9 fold increased), followed by lanosterol (1.2/3 fold increased) and dehydrodesmosterol (1.2/2.9 fold increased). Of note, while this method was able to detect corresponding sterol intermediates of the Kandutsch-Russel Pathway (dihydrolanosterol, dihydro-FF-MAS or 7-dehydrocholesterol), they were not detected in this analysis. Taken together these results suggest that (S)-crizotinib and AY9944 inhibit the activity of multiple dehydrocholesterol reductases (DHCR7, DHCR14, DHCR24) and lanosterol 14 alpha-demethylase (CYP51A1), resulting in the accumulation of lanosterol (DHCR24+CYP51A1), FF-MAS (DHCR24+DHCR14) and 7-dehydrodesmosterol (DHCR24+DHCR7). The accumulation of sterol intermediates in the Bloch Pathway suggests that (S)-crizotinib and AY9944 effectively inhibit DHCR24 at a concentration of 10 μM, with (S)-crizotinib primarily inhibiting the enzymatic activities of CYP51A1 and DHCR24, and AY9944 primarily inhibiting the enzymatic activities of DHCR14 (present in Tm7sf2 and LBR) and DHCR24. Thus, the antimicrobial activities of (S)-crizotinib and AY9944 are associated with the inhibition of dihydrocholesterol reductases and lanosterol 14 alpha-demethylase resulting in the accumulation of lanosterol, FF-MAS and 7-dehydrodesmosterol. Of note, the accumulation of lanosterol was recently found to be associated with increased antimicrobial activity against intracellular Listeria²⁷, which may suggest that the inhibition of DHCR24 and CYP51A1 by crizotinib and related compounds may be inducing antimicrobial activity.

Various modifications and variations of the described compounds, pharmaceutical compositions, kits, and methods of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### References

1. Antimicrobial resistance: global report on surveillance. (2014).

2. Lewis, K. The Science of Antibiotic Discovery. Cell 181, 29-45 (2020).

3. Grant, S. S. & Hung, D. T. Persistent bacterial infections, antibiotic tolerance, and the oxidative stress response. Virulence 4, 273-283 (2013).

4. Fisher, R. A., Gollan, B. & Helaine, S. Persistent bacterial infections and persister cells. Nat. Rev. Microbiol. 15, 453-464 (2017).

5. Lewis, K. Persister Cells. Annu. Rev. Microbiol. 64, 357-372 (2010).

6. Centers for Disease Control and Prevention (U.S.). Antibiotic resistance threats in the United States, 2019. (2019) doi: 10.15620/cdc:82532.

7. Magill, S. S. et al. Multistate Point-Prevalence Survey of Health Care-Associated Infections. N. Engl. J. Med. 370, 1198-1208 (2014).

8. Hunstad, D. A. & Justice, S. S. Intracellular lifestyles and immune evasion strategies of uropathogenic Escherichia coli. Anmi. Rev. Microbiol. 64, 203-221 (2010).

9. O'Brien, V. P., Hannan, T. J., Schaeffer, A. J. & Hultgren, S. J. Are you experienced? Understanding bladder innate immunity in the context of recurrent urinary tract infection. Curr. Opin. Infect. Dis. 28, 97-105 (2015).

10. Ernst, C. M. et al. Adaptive evolution of virulence and persistence in carbapenem-resistant Klebsiella pneumoniae. Nat. Med. 26, 705-711 (2020).

11. Balaban, N. Q. Bacterial Persistence as a Phenotypic Switch. Science 305, 1622-1625 (2004).

12. Moldoveanu, A. L., Rycroft, J. A. & Helaine, S. Impact of bacterial persisters on their host. Curr. Opin. Microbiol. 59, 65-71 (2021).

13. Huber, K. V. M. et al. Stereospecific targeting of MTH1 by (S)-crizotinib as an anticancer strategy. Nature 508, 222-227 (2014).

14. Shaw, A. T., Yasothan, U. & Kirkpatrick, P. Crizotinib. Nat. Rev. Drug Discov. 10, 897-898 (2011).

15. Samaranayake, G., Huynh, M. & Rai, P. MTHI as a Chemotherapeutic Target: The Elephant in the Room. Cancers 9, 47 (2017).

16. Miao, Y., Li, G., Zhang, X., Xu, H. & Abraham, S. N. A TRP Channel Senses Lysosome Neutralization by Pathogens to Trigger Their Expulsion. Cell 161, 1306-1319 (2015).

17. Hannan, T. J. et al. Host-pathogen checkpoints and population bottlenecks in persistent and intracellular uropathogenic Escherichia coli bladder infection. FEMS Microbiol. Rev. 36, 616-648 (2012).

18. Hannan, T. J., Mysorekar, I. U., Hung, C. S., Isaacson-Schmid, M. L. & Hultgren, S. J. Early Severe Inflammatory Responses to Uropathogenic E. coli Predispose to Chronic and Recurrent Urinary Tract Infection. PLoS Pathog. 6, e1001042 (2010).

19. Cui, J. J. et al. Structure based drug design of crizotinib (PF-02341066), a potent and selective dual inhibitor of mesenchymal-epithelial transition factor (c-MET) kinase and anaplastic lymphoma kinase (ALK). J. Med. Chem. 54, 6342-6363 (2011).

20. Chen, L. et al. Molecular basis for receptor tyrosine kinase A-loop tyrosine transphosphorylation. Nat. Chem. Biol. 16, 267-277 (2020).

21. Farand, J. et al. Discovery of Potent and Selective MTH1 Inhibitors for Oncology: Enabling Rapid Target (In) Validation. ACS Med. Chem. Lett. 11, 358-364 (2020).

22. Zheng, Y. -D. et al. Crizotinib Shows Antibacterial Activity against Gram-Positive Bacteria by Reducing ATP Production and Targeting the CTP Synthase PyrG. Microbiol. Spectr. 17.

23. Huber, K. V. M. et al. Proteome-wide drug and metabolite interaction mapping by thermal-stability profiling. Nat. Methods 12, 1055-1057 (2015).

24. Kettle, J. G. et al. Potent and Selective Inhibitors of MTH1 Probe Its Role in Cancer Cell Survival. J. Med. Chem. 59, 2346-2361 (2016).

25. Horling, A., Müller, C., Barthel, R., Bracher, F. & Imming, P. A new class of selective and potent 7-dehydrocholesterol reductase inhibitors. J. Med. Chem. 55, 7614-7622 (2012).

26. Fernández, C., Martín, M., Gómez-Coronado, D. & Lasunción, M. A. Effects of distal cholesterol biosynthesis inhibitors on cell proliferation and cell cycle progression. J. Lipid Res. 46, 920-929 (2005).

27. Araldi, E. et al. Lanosterol Modulates TLR4-Mediated Innate Immune Responses in Macrophages. Cell Rep. 19, 2743-2755 (2017).

What is claimed is:

1. A compound or a salt thereof, wherein the compound has the structural formula: 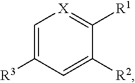
wherein X is C or N;
wherein R¹ is a hydrogen group, an amino group, an alkyl (including cycloalkyl) amino group, an alkyl azide (including a cycloalkyl azide) group, an alkylaryl (including cycloalkylaryl) amino group, an alkylaryloxy (including cycloalkylaryloxy) group, or an aryl amino group, and wherein R¹ is optionally independently substituted at one or more aryl ring positions with one or more halogen groups;
wherein R² is a hydrogen group, a hydroxyl group, an alkoxy (including cycloalkoxy) group, an alkynyl alkoxy (including alkynyl cycloalkoxy) group, or an aryl alkoxy (including aryl cycloalkoxy) group, and wherein, optionally, the aryl alkoxy (including aryl cycloalkoxy) group is independently substituted at one or more aryl ring positions with one or more halogen groups;
wherein R³ is a halogen group or a ring carbon of an aromatic heterocyclic amine group, wherein, optionally, the aromatic heterocyclic amine group is independently substituted at one or more heterocyclic ring positions with one or more alkyl (including cycloalkyl) groups, alkoxycarbonyl (including cycloalkoxycarbonyl) groups, carboxylic acid groups, and/or nonaromatic heterocyclic amine groups; and
wherein the compound excludes (R)-Crizotinib or (S)-Crizotinib.

2. The compound of claim 1, wherein the aryl alkoxy group of R² is a hydrogen group, a C₄-C₆ alkyl ether group, a benzyloxy group, a 2-(phenyl)ethoxy group, or a chiral 1-(phenyl)ethoxy group, and wherein R² is optionally independently substituted at one or more aryl ring positions with one or more fluorine groups and/or one or more chlorine groups, optionally wherein the aryl alkoxy group of R² is a 1-(2,6-dichloro-3-fluorophenylethoxy) group comprising an (R) configuration, a 2-(2,6-dichloro-phenylethoxy) group, or a (2,6-dichloro-3-fluorobenzyloxy) group.

3. The compound of claim 1, wherein the aromatic heterocyclic amine group of R³ is a pyrazole group, a pyrrole group, or a pyrimidine group, optionally wherein the aromatic heterocyclic amine group R³ is a 1-(piperidin-4-yl)pyrazol-4-yl group, a 1-(N-alkylpiperidin-4-yl)pyrazol-4-yl group, or a 1-(N-alkylalkynypiperidin-4-yl)pyrazol-4-yl group.

4. The compound of claim 1, wherein the nonaromatic heterocyclic amine group of R³ is substituted at one or more positions with one or more piperidine groups optionally independently substituted at one or more positions with one or more formyl groups, alkyl (including cycloalkyl) groups, alkylalkynyl (including cycloalkylalkynyl) groups, and/or alkoxycarbonyl (including cycloalkycarbonyl) groups.

5. The compound of claim 1, wherein the compound is selected from the group consisting of:

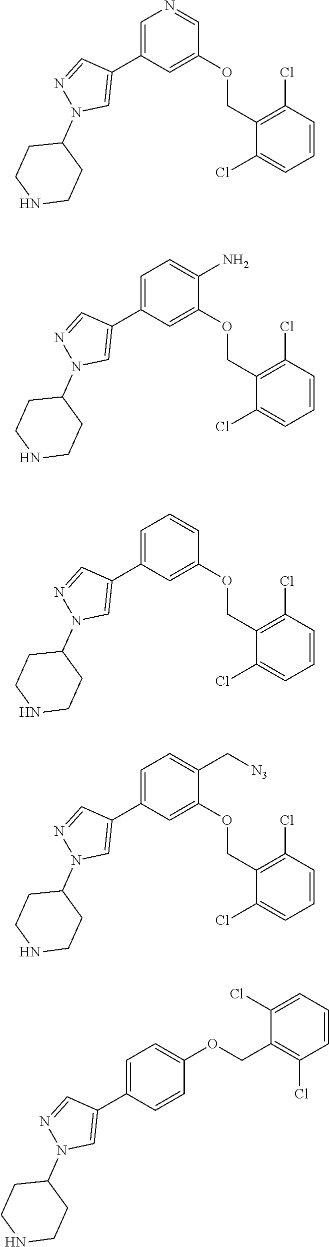 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

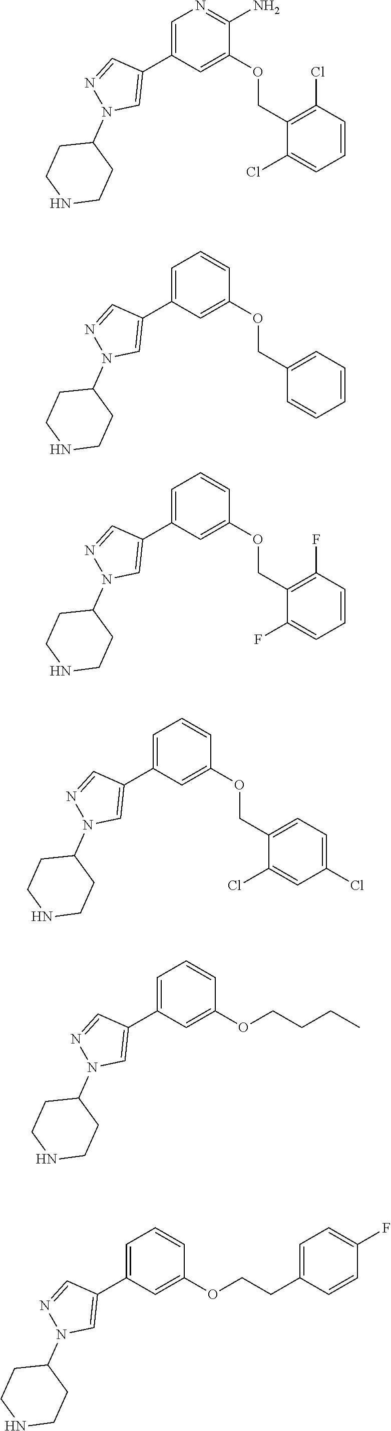 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

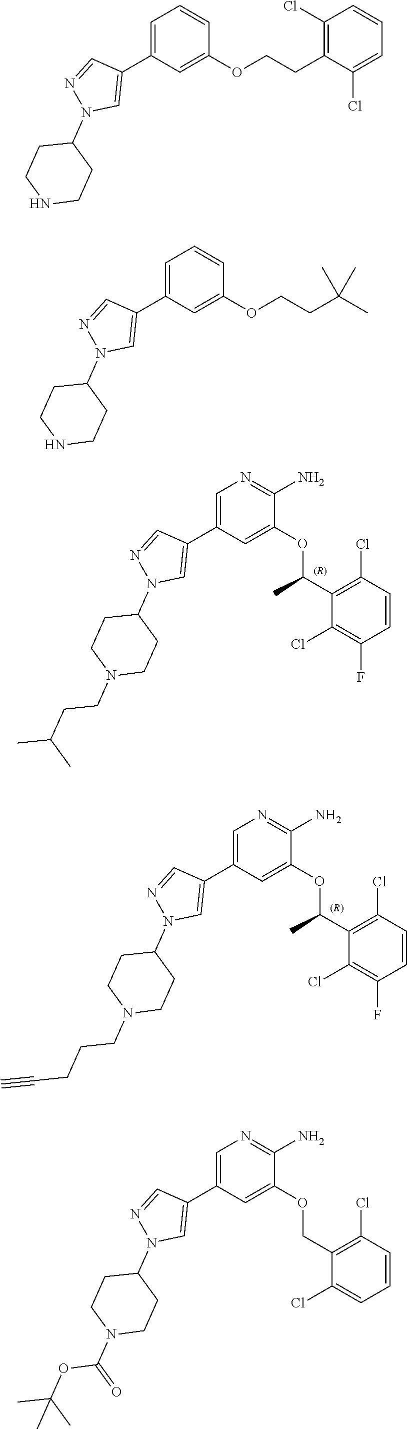

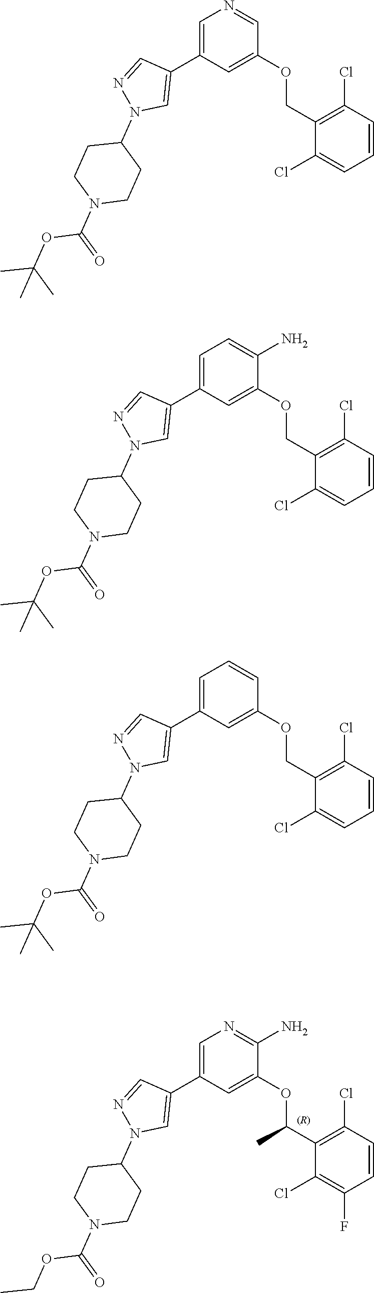

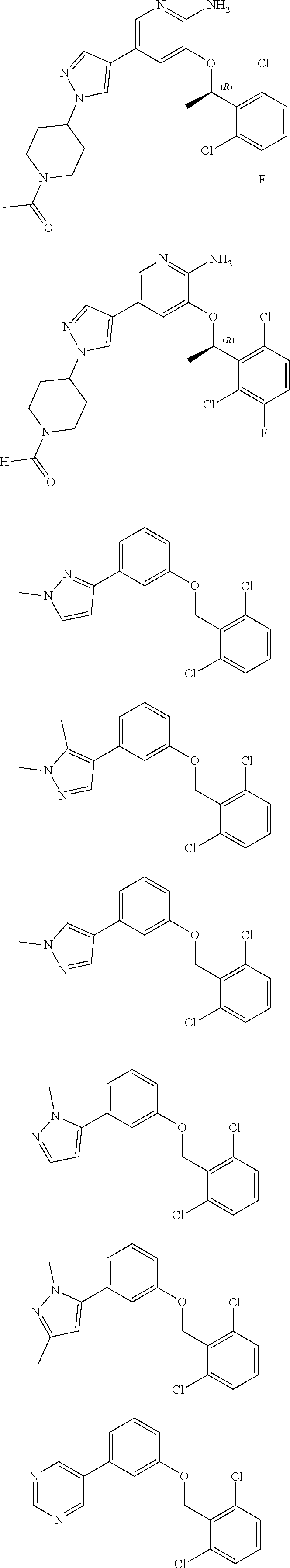

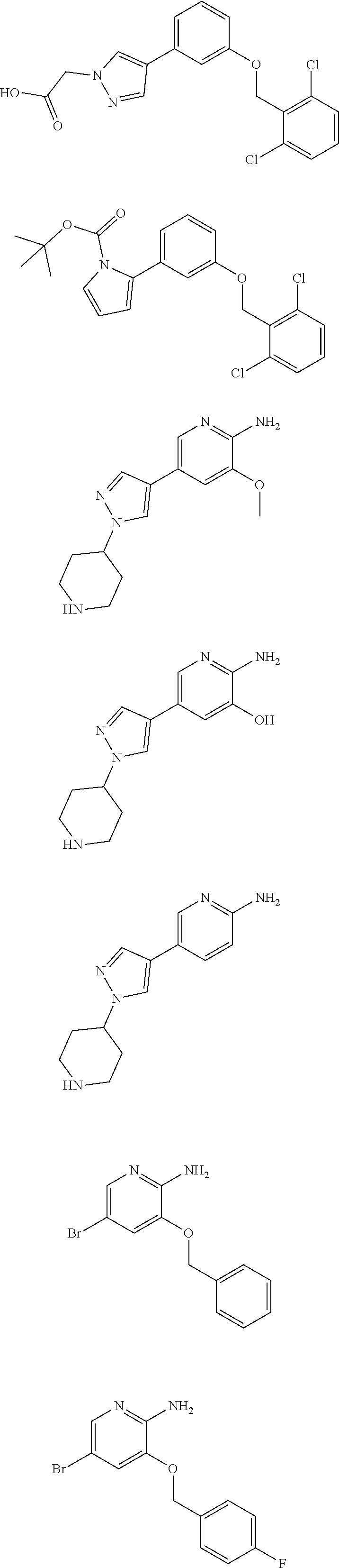

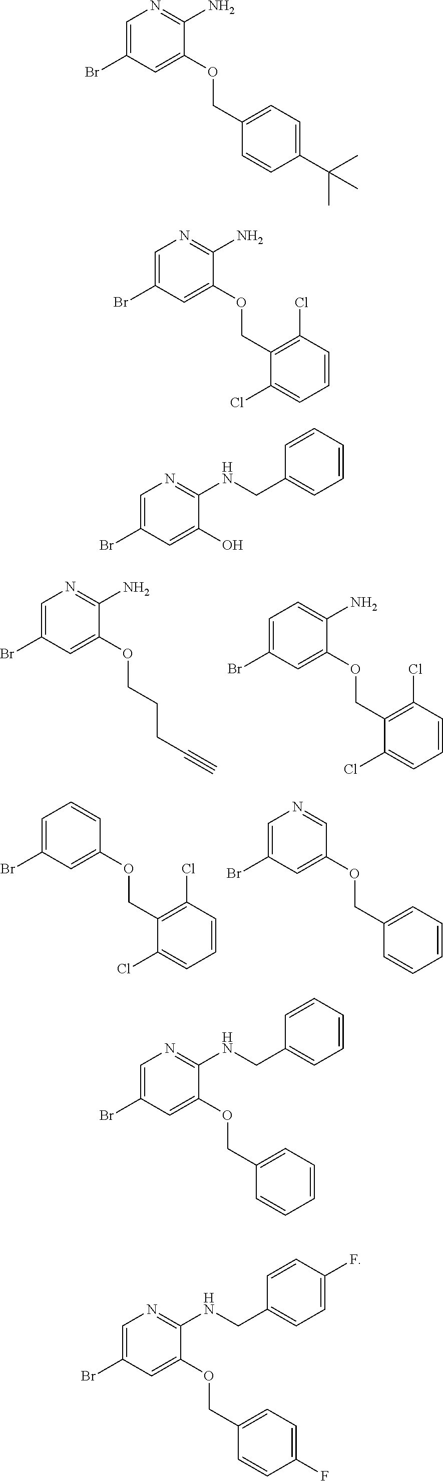

6. The compound of claim 1, wherein the compound is selected from the group consisting of:

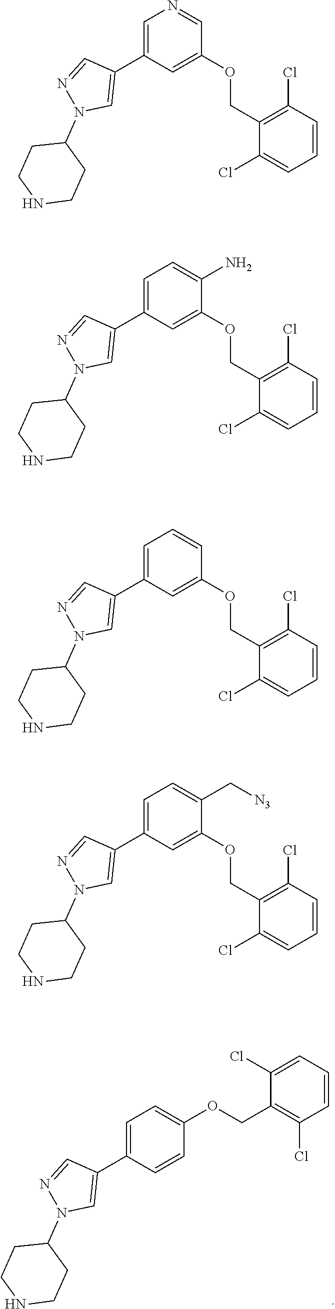 [SMILES: Clc1cccc(Cl)c1COc1cncc(-c2cnn(C3CCNCC3)c2)c1 ; Nc1ccc(-c2cnn(C3CCNCC3)c2)cc1OCc1c(Cl)cccc1Cl ; Clc1cccc(Cl)c1COc1cccc(-c2cnn(C3CCNCC3)c2)c1 ; [N-]=[N+]=NCc1ccc(-c2cnn(C3CCNCC3)c2)cc1OCc1c(Cl)cccc1Cl ; Clc1cccc(Cl)c1COc1ccc(-c2cnn(C3CCNCC3)c2)cc1]

7. The compound of claim 1, wherein the compound is selected from the group consisting of:

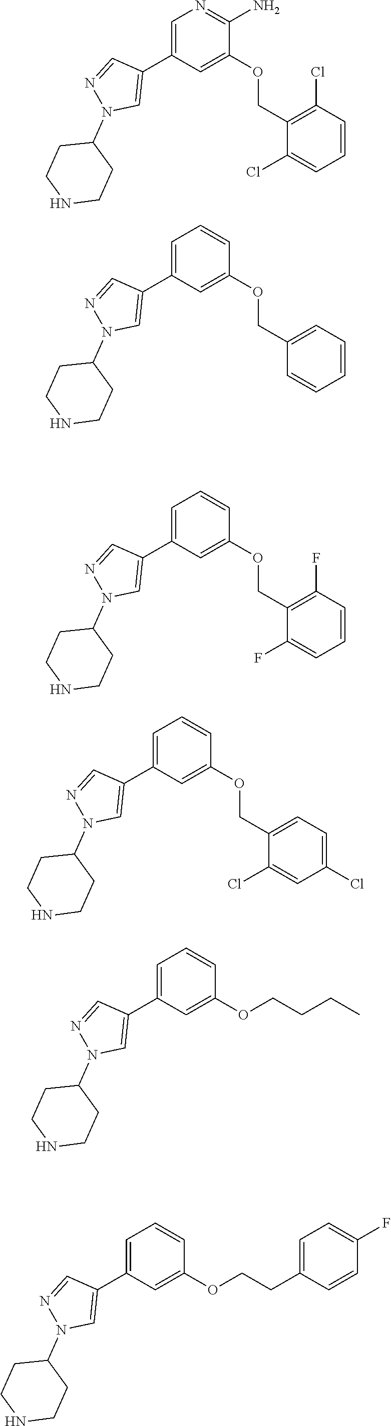 [SMILES: Nc1ncc(-c2cnn(C3CCNCC3)c2)cc1OCc1c(Cl)cccc1Cl ; c1ccc(COc2cccc(-c3cnn(C4CCNCC4)c3)c2)cc1 ; Fc1cccc(F)c1COc1cccc(-c2cnn(C3CCNCC3)c2)c1 ; Clc1ccc(COc2cccc(-c3cnn(C4CCNCC4)c3)c2)c(Cl)c1 ; CCCCOc1cccc(-c2cnn(C3CCNCC3)c2)c1 ; Fc1ccc(CCOc2cccc(-c3cnn(C4CCNCC4)c3)c2)cc1]

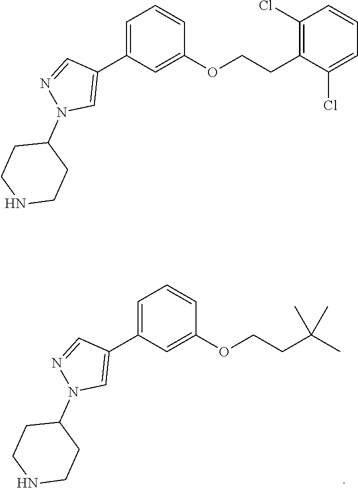 [SMILES: Clc1cccc(Cl)c1CCOc1cccc(-c2cnn(C3CCNCC3)c2)c1 ; CC(C)(C)CCOc1cccc(-c2cnn(C3CCNCC3)c2)c1]

8. The compound of claim 1, wherein the compound is selected from the group consisting of:

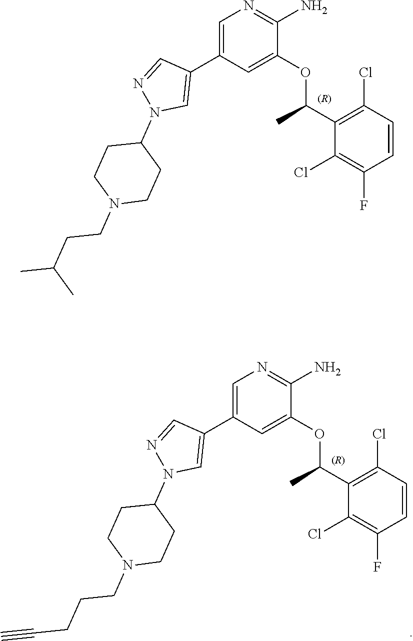

9. The compound of claim 1, wherein the compound is selected from the group consisting of:

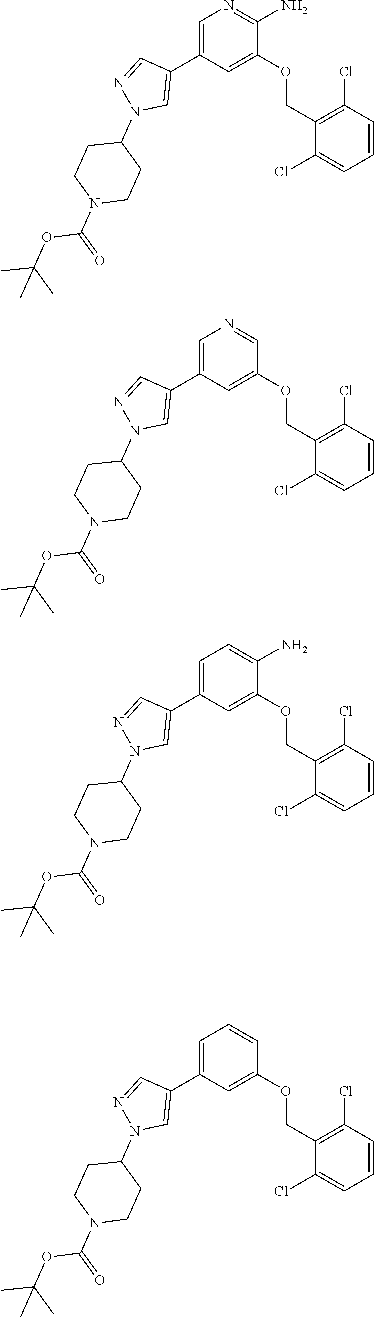 [SMILES: CC(C)(C)OC(=O)N1CCC(n2cc(-c3cnc(N)c(OCc4c(Cl)cccc4Cl)c3)cn2)CC1 ; CC(C)(C)OC(=O)N1CCC(n2cc(-c3cncc(OCc4c(Cl)cccc4Cl)c3)cn2)CC1 ; CC(C)(C)OC(=O)N1CCC(n2cc(-c3ccc(N)c(OCc4c(Cl)cccc4Cl)c3)cn2)CC1 ; CC(C)(C)OC(=O)N1CCC(n2cc(-c3cccc(OCc4c(Cl)cccc4Cl)c3)cn2)CC1]

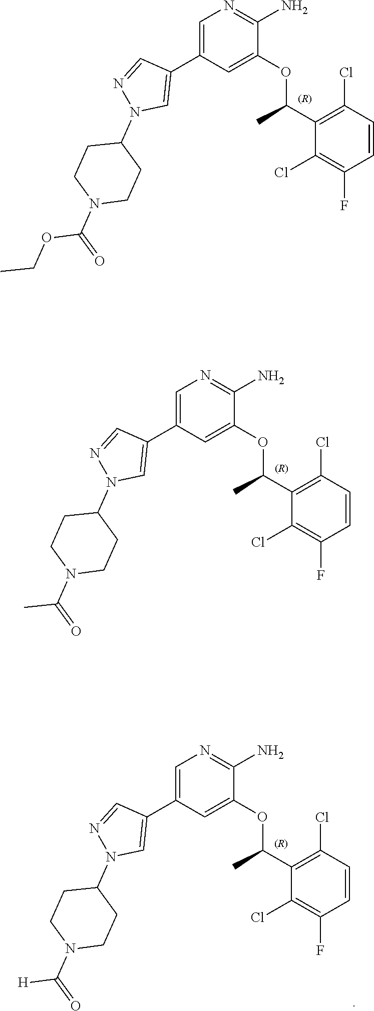

10. The compound of claim 1, wherein the compound is selected from the group consisting of:

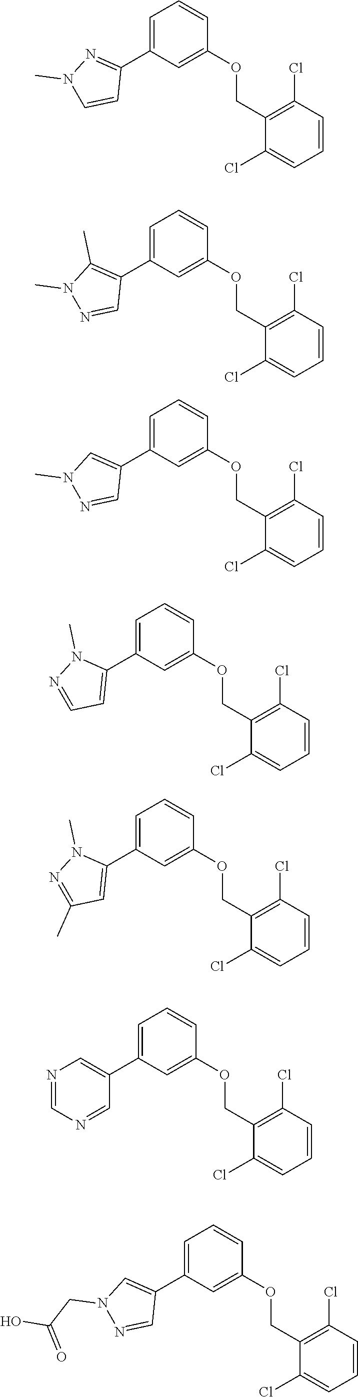

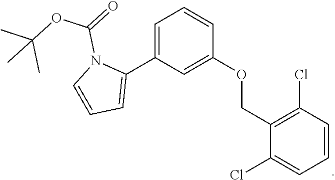 [SMILES: CC(C)(C)OC(=O)n1cccc1-c1cccc(OCc2c(Cl)cccc2Cl)c1]

11. The compound of claim 1, wherein the compound is selected from the group consisting of:

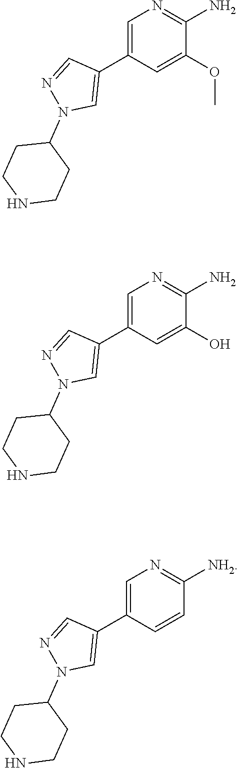 [SMILES: COc1cc(-c2cnn(C3CCNCC3)c2)cnc1N ; Nc1ncc(-c2cnn(C3CCNCC3)c2)cc1O ; Nc1ccc(-c2cnn(C3CCNCC3)c2)cn1]

12. The compound of claim 1, wherein the compound is selected from the group consisting of:

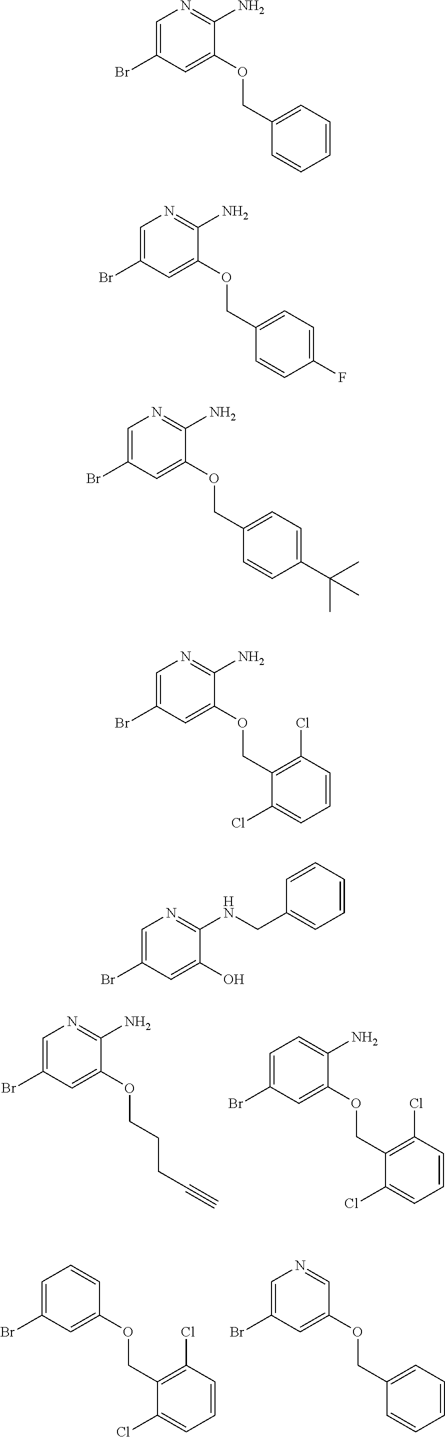

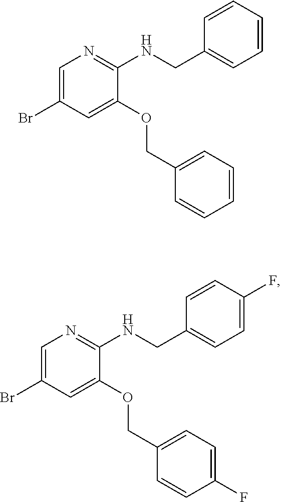 [SMILES: Brc1cnc(NCc2ccccc2)c(OCc2ccccc2)c1 ; Fc1ccc(CNc2ncc(Br)cc2OCc2ccc(F)cc2)cc1]

13. A pharmaceutical formulation comprising an effective amount of a compound of claim 1 or a pharmaceutically acceptable salt thereof, optionally in combination with one or more compounds selected from (R)-Crizotinib, (S)-Crizotinib, and AY 9944, in a pharmaceutically acceptable carrier.

14. A method for treating or preventing a bacterial infection or for inducing antimicrobial activity against a bacterial infection, comprising: administering to a subject in need thereof a therapeutically effective amount of a compound of claim 1 or a pharmaceutically acceptable salt thereof, optionally in combination with one or more compounds selected from (R)-Crizotinib, (S)-Crizotinib, and AY 9944.

15. The method of claim 14, wherein the bacterial infection is an acute bacterial infection, a chronic bacterial infection, a latent bacterial infection, a slow bacterial infection, a persistent bacterial infection, an antibiotic resistant bacterial infection, a dormant bacterial infection, a bacterial infection present in biofilm or any combination thereof.

16. The method of claim 14, wherein the bacterial infection is a central nervous system infection, an eye infection, an ear infection, an upper respiratory tract infection, a lower respiratory tract infection, gastrointestinal infection, a heart infection, a gallbladder infection, a urinary tract infection, a skin infection, a blood infection, a bone infection, a hospital-acquired infection, a wound infection, a vaginal infection, a sexually transmitted disease, or any combination thereof.

17. A method for treating or preventing a viral infection or for inducing antimicrobial activity against a viral infection comprising: administering to a subject in need thereof a therapeutically effective amount of a compound of claim 1 or a pharmaceutically acceptable salt thereof, optionally in combination with one or more compounds selected from (R)-Crizotinib, (S)-Crizotinib, and AY 9944.

18. The method of claim 17, wherein the viral infection is an acute viral infection, a chronic viral infection, a latent viral infection, a slow viral infection, a persistent viral infection, an antimicrobial resistant viral infection, dormant viral infection or any combination thereof.

19. The method of claim 17 wherein the viral infection is caused by a Coronaviridae virus.

20. The method of claim 19, wherein the Coronaviridae virus is a from β-coronavirus, optionally wherein the β-coronavirus is SARS-COV-2.
